(19) United States
(12) Patent Application Publication
(10) Pub. No.: US 2011/0092554 A1
(43) Pub. Date: Apr. 21, 2011

(54) 1,3,5 TRI-SUBTITUTED BENZENES FOR TREATMENT OF ALZHEIMER'S DISEASE AND OTHER DISORDERS

(76) Inventors: Richard Chesworth, Boston, MA (US); Gideon Shapiro, Gainesville, FL (US)

(21) Appl. No.: 12/743,678

(22) PCT Filed: Nov. 19, 2008
(86) PCT No.: PCT/US08/83998

§ 371 Date: Dec. 1, 2010

Related U.S. Application Data
(60) Provisional application No. 60/989,096, filed on Nov. 19, 2007.

(51) Int. Cl. A61K 31/433; C07D 271/12; A61K 31/4245; A61P 25/28

(52) U.S. Cl. 514/362; 548/126; 514/361

(57) ABSTRACT

The present disclosure relates to novel 1,3,5 tri-substituted benzenes of general formula (I), (II) or (III) and the use of such compounds in the treatment of diseases associated with the deposition of -amyloid in the brain.

### BACKGROUND

Alzheimer's disease (AD) is the most prevalent form of dementia. It is a neurodegenerative disorder that is associated (though not exclusively) with aging. The disorder is clinically characterized by a progressive loss of memory, cognition, reasoning and judgment that leads to an extreme mental deterioration and ultimately death. The disorder is pathologically characterized by the deposition of extracellular plaques and the presence of neurofibrillary tangles. These plaques are considered to play an important role in the pathogenesis of the disease.

These plaques mainly comprise of fibrillar aggregates of β-amyloid peptide (Aβ), which are products of the amyloid precursor protein (APP), a 695 amino-acid protein. APP is initially processed by β-secretase forming a secreted peptide and a membrane bound C99 fragment. The C99 fragment is subsequently processed by the proteolytic activity of γ-secretase. Multiple sites of proteolysis on the C99 fragment lead to the production of a range of smaller peptides (Aβ37-42 amino acids). N-terminal truncations can also be found e.g. Aβ (4-42) for convenience Aβ40 and Aβ42 as used herein incorporates these N-terminal truncated peptides. Upon secretion, the Aβ peptides initially form soluble aggregates which ultimately lead to the formation of insoluble deposits and plaques. Aβ42 is believed to be the most neurotoxic, the shorter peptides have less propensity to aggregate and form plaques. The Aβ plaques in the brain are also associated with cerebral amyloid angiopathy, hereditary cerebral hemorrhage with amyloidosis, multi infarct dementia, dementia pugilistisca and Down's Syndrome.

γ-secretase is an association of proteins, comprising Aph1, Nicastrin, Presenillin and Pen-2 (review De Strooper 2003, Neuron 38, 9). Aβ42 is selectively increased in patients carrying particular mutations in a protein presenilin. These mutations are correlated with early onset a familial AD. Inhibition of γ-secretase resulting in the lowering of Aβ42 is a desirable activity for the pharmaceutical community and numerous inhibitors have been found e.g. Thompson et at (Bio. Org. and Med. Chem. Letters 2006, 16, 2357-63), Shaw et at (Bio. Org. and Med. Chem. Letters 2006, 17, 511-16) and Asberom et al (Bio. Org. and Med. Chem. Letters 2007, 15, 2219-2223). Inhibition of γ-secretase though is not without side-effects, some of which are due to the γ-secretase complex processing substrates other than C99, for e.g. Notch. A more desirable approach is to modulate the proteolytic activity of the γ-secretase complex in a manner that lowers Aβ42 in favor of shorter peptides without affecting the activity of γ-secretase on substrates such as Notch.

Compounds that have shown modulation of γ-secretase include certain non-steroidal, anti-inflammatory drugs (NSAIDs), for example Flurbiprofen, (Stock et at Bio. Org. and Med. Chem. Letters 2006, 16, 2219-2223). Other publications that disclose agents said to reduce Aβ42 through the modulation of γ-secretase include WO 04/074232, WO 05/054193, Perreto et at Journal of Medicinal Chemistry 2005, 48 5705-20, WO05/108362, WO 06/008558, WO 06/021441, WO 06/041874, WO 06/045554, WO04110350, WO 06/043964, WO 05/115990, EP1847524, WO 07/116,228, WO 07/110,667 and WO 07/124,394.

### DESCRIPTION OF THE DISCLOSURE

In a first embodiment compounds of formula (I), (II) and (III) are disclosed

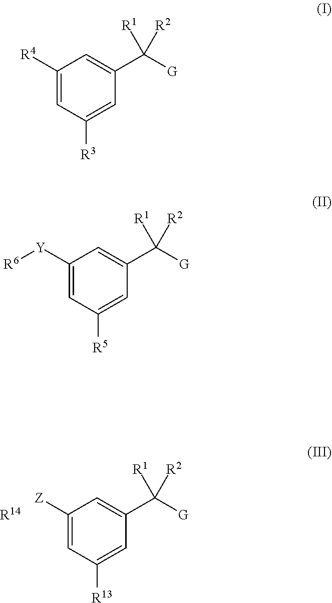

where G is a carboxylic acid or a tetrazole;

R¹ and R² are independently selected from H or R¹⁵;

Or

R¹ and R² are taken together to form a mono or bicyclic ring system having 4 to 11 ring atoms selected from C, N, O and S, provided that not more than 3 ring atoms in any single ring are other than C; and optionally independently singly or multiply substituted with one or more substituents selected from, halogen, hydroxyl, amino, cyano or a C₁₋₄ alkyl substituent

Or

R₁ and R₂ are taken together to form a 3-7 membered cycloalkyl ring substituted with R₂₅ and R₂₆ where R₂₅ and R₂₆ are attached to the same carbon and taken together to form a second 3-7 membered cycloalkyl ring wherein each cycloalkyl is optionally multiply and independently substituted with halo, hydroxy, cyano, CF₃, C₁-C₄ alkyl (for example 5, 5 spiro[2.3]hexyl system)

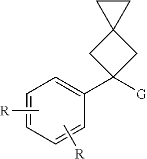

R¹⁵ is selected from C₃-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl; wherein R¹⁵ is optionally substituted with one or more substituents independently selected from the group consisting of halo, N₃, CN, NO₂, oxo, OH, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R)CO₂R¹¹; OC(O)N(R¹¹R¹²);

R³ is aryl and is optionally substituted with one or more substituents independently selected from halo, N₃, CN, NO₂, OH, R⁹, OR⁹, SR⁹, S(O)R⁹, SO₂R⁹, CO₂R⁹, OC(O)R⁹, C(O)R⁹; C(O)N(R⁹R¹¹); C(O)NH(R¹¹); C(O)NH(R⁹); SO₂N(R⁹R¹¹); SO₂NH(R⁹); SO₂NH(R¹¹); S(O)N(R⁹R¹¹); S(O)NH(R⁹); S(O)NH(R¹¹); NHSO₂R¹¹; N(R⁹)SO₂R¹¹; NHSOR11; N(R⁹)SOR¹¹; N(R⁹)SO₂N(R¹⁰R¹¹); NHSO₂N(R¹⁰R¹¹); N(R⁹)SO₂NH(R¹¹); N(R⁹)SO₂NH(R¹¹); N(R⁹R¹¹); NH(R⁹); NH(R¹¹); N(R⁹)C(O)R¹¹; NHC(O)R¹¹; N(R⁹)C(O)N(R¹¹R¹²); NHC(O)N(R¹¹R¹²); N(R⁹)C(O)NH(R¹¹); N(R⁹)C(O)NH(R¹²); N(R⁹)CO₂R¹¹; NHCO₂R¹¹; OC(O)N(R¹¹R¹²); OC(O)NH(R¹¹); OC(O)NH(R¹²);

R⁴ is selected from, C₁-C₆ alkyl, C₁-C₆ alkoxy, —O—(C₂-C₆ alkyl)-OH, —O—(C₂-C₆ alkyl)-O—(C₁-C₆ alkyl), heteroaryl, C₃-C₇ cycloalkyl, C₁-C₆ alkynyl heterocycyl, —O—(C₁-C₄ alkyl)-Het² or R⁷—X—; wherein X is selected from —C₁-C₆ alkyl, —(C₀-C₆ alkyl)-O—(C₁-C₄ alkyl)-, —C(O)—, S(O)p-, —C(O)NR⁸—, N(R⁸)—C(O)—, —SO₂N(R⁸)—, —N(R⁸)—SO₂—, —O—C(O)NR⁸—, —N(R⁸)—C(O)—O—, —N(R⁸)—C(O)NR⁸—, —N(R⁸)—C(O)—N(R⁸)—, —C(O)—O—, —O—C(O)—, —O—C(O)—O—, where the leftmost radical is attached to R⁷ and each alkyl group is optionally multiply substituted with groups independently selected from halo, —CF₃, —OCF₃, hydroxyl, amino, oxo and cyano;

p is an integer selected from 1 and 2;

R⁷ is selected from C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl,

wherein R⁴ and R⁷ are independently and optionally multiply substituted with halo, N₃, CN, NO₂, OH, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R⁹)CO₂R¹¹; OC(O)N(R¹¹R¹²);

R⁸ is selected from H, C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl, and R⁸ is optionally multiply substituted with groups independently selected from halo, —CF₃, —OCF₃, hydroxyl, amino, oxo or cyano;

R⁹ is selected from the following groups:

C₁-C₇-alkyl, C₃-C₇ saturated cycloalkyl, (C₁-C₃)alkyl-(C₃-C₇)cycloalkyl, C₃-C₇ partially unsaturated cycloalkyl, saturated 4-8 membered heterocycle, partially unsaturated 4-8 membered heterocycle phenyl, heteroaryl, C₁-C₇-alkoxy and O—C₂-C₇—O—C₁-C₄ each of which is optionally with one or more substituents independently selected from the group F, CI, Br, I, CF₃, CN, OH, oxo, NH₂, NR¹¹R¹²;

R¹⁰, R¹¹, R¹² are independently selected from the group consisting of C₁-C₇ alkyl, C₁-C₇ alkoxy, O—C₂-C₇—O—C₁₋₄, 4-8 membered heterocycle; and C₃-C₇ cycloalkyl, phenyl or heteroaryl;

each R¹⁰, R¹¹, R¹² group is optionally substituted with one or more substituents independently selected from the group consisting of F, CI, Br, I, CN, OH, oxo, amino and CF₃;

R⁵ is selected from heteroaryl, C₃-C₇ cycloalkyl, and heterocycyl,

R⁵ is optionally substituted with one or more substituents independently selected from the group consisting of halo, N₃, CN, NO₂, OH, oxo, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R⁹)CO₂R¹¹; OC(O)N(R¹¹R¹²);

Where Y is selected from a covalent bond, —O—, —C₁-C₆ alkyl, O—(C₁-C₆ alkyl)-, —(C₁-C₆ alkyl)-O—, —(C₁-C₆ alkyl)-O—(C₁-C₆ alkyl)-, —C(O)—, S(O)ₚ—, —O—C(R)(R)—, —C(O)NR⁸—, N(R⁸)—C(O)—, —SO₂N(R⁸)—, —N(R⁸)—SO₂—, —O—C(O)NR⁸—, —N(R)—C(O)—O—, —N(R⁸)—C(O)NR⁸—, —N(R⁸)—C(O)—N(R⁸)—, —C(O)—O—, —O—C(O)—, —O—C(O)—O—, where the leftmost radical is attached to R⁶;

p is 0, 1 or 2;

each alkyl group is optionally multiply substituted with groups independently selected from halo, hydroxyl, amino, cyano oxo, and CF₃;

R⁶ is selected from C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl;

R⁶ is optionally substituted with one or more substituents independently selected from the group consisting of halo, N₃, CN, NO₂, oxo, OH, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R⁹)CO₂R¹¹; OC(O)N(R¹¹R¹²);

R¹³ is selected from halo, CN, CF₃, OCF₃, C₁-C₇ alkyl, C₁₋₇ alkoxy, —O—(C₂-C₇-alkyl)-O—C₁₋₄ alkyl), —O—(C₁-C₄ alkyl)-(C₃-C₇)cycloalkyl and —(C₁-C₄ alkyl)-cycloalkyl each R¹³ is optionally multiply substituted with halo, cyano, CF₃ hydroxyl, oxo and amino;

R¹⁴ is selected from aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl;

R¹⁴ is optionally substituted with one or more substituents independently selected from the group consisting of halo, N₃, CN, NO₂, OH, oxo, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R⁹)CO₂R¹¹; OC(O)N(R¹¹R¹²);

Where Z is selected from —O—, —C₁-C₆ alkyl, O—(C₁-C₆ alkyl)-, —(C₁-C₆ alkyl)-O—, —(C₁-C₆ alkyl)-O—(C₁-C₆ alkyl)-, —C(O)—, S(O)ₚ—, —C(O)NR⁸—, N(R⁸)—C(O)—, —SO₂N(R⁸)—, —N(R⁸)—SO₂—, —O—C(O)NR⁸—, —N(R)—C(O)—O—, —N(R⁸)—C(O)NR⁸—, —N(R⁸)—C(O)—N(R⁸)—, —C(O)—O—, —O—C(O)—, —O—C(O)—O—, where the leftmost radical is attached to R¹⁴; and p is 0, 1 or 2.

In certain embodiments of each of Formulas (I), (II) and (III) R¹ is H and R² is R¹⁵.

In certain embodiments of each of Formulas (I), (II) and (III) R¹⁵ is optionally multiply and independently substituted with hydroxy, oxo, fluoro, methoxy, ethoxy, thiomethyl and thioethyl.

In certain embodiments of each of Formulas (I), (II) and (III) R¹⁵ is unsubstituted.

In certain embodiments of each of Formulas (I), (II) and (III) R⁹ is selected from the following groups C₁-C₇-alkyl, C₃-C₇ saturated cycloalkyl, (C₁-C₃)alkyl-(C₃-C₇)cycloalkyl and C₁-C₇-alkoxy each of which is optionally with one or more substituents independently selected from the group F, CI, Br, I, CF₃, CN, OH or oxo.

In a another embodiment a compound of formula (I) is selected:

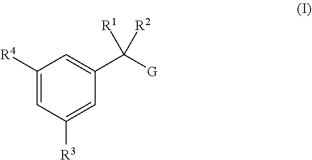

In another embodiment a compound of formula (I) is selected where G is a carboxylic acid.

In another embodiment a compound of formula (I) is selected where G is a tetrazole.

In another embodiment a compound of formula (I) is selected where R¹ and R² are independently selected from H or R¹⁵.

In another embodiment a compound of formula (I) is selected where R¹ and R² when taken together to form a mono or bicyclic ring system comprising of 4 to 11 ring atoms selected from C, N, O and S provided that not more than 3 ring atoms in any single ring are other than C and each ring is optionally independently singly or multiply substituted with one or more substituents selected from, halogen, hydroxyl, amino, cyano or a C₁₋₄ alkyl substituent.

In another embodiment a compound of formula (I) is selected where R₁ and R₂ are taken together to form a 3-7 membered cycloalkyl ring substituted with R₂₅ and R₂₆ where R₂₅ and R₂₆ are attached to the same carbon and taken together to form a second 3-7 membered cycloalkyl ring wherein each cycloalkyl is optionally multiply and independently substituted with halo, hydroxy, cyano, CF₃, C₁-C₄ alkyl.

For example 5,5-spiro[2.3]hexyl system

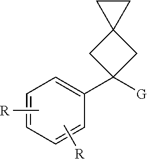

In another embodiment a compound of formula (I) is selected where R¹⁵ is C₃-C₆ alkyl.

In another embodiment a compound of formula (I) is selected where R¹⁵ is C₁-C₆ alkoxy.

In another embodiment a compound of formula (I) is selected where R¹⁵ is —O—(C₂-C₆ alkyl)-OH.

In another embodiment a compound of formula (I) is selected where R¹⁵ is —O—(C₂-C₆ alkyl)-O—(C₁-C₆ alkyl).

In another embodiment a compound of formula (I) is selected where R¹⁵ is aryl.

In another embodiment a compound of formula (I) is selected where R¹⁵ is, —(C₁-C₄ alkyl)-aryl.

In another embodiment a compound of formula (I) is selected where R¹⁵ is heteroaryl.

In another embodiment a compound of formula (I) is selected where R¹⁵ is —(C₁-C₄ alkyl)-heteroaryl.

In another embodiment a compound of formula (I) is selected where R¹⁵ is C₃-C₇ cycloalkyl.

In another embodiment a compound of formula (I) is selected where R¹⁵ is —(C₁-C₄ alkyl)-(C₃-C₇) cycloalkyl.

In another embodiment a compound of formula (I) is selected where R¹⁵ is heterocycyl

In another embodiment a compound of formula (I) is selected where R¹⁵ is —(C₁-C₄ alkyl)-heterocycyl.

R¹⁵ is optionally substituted with one or more substituents independently selected from the group consisting of halo, N₃, CN, NO₂, oxo, OH, R⁹, OR⁹, SR⁹, S(O)R⁹, SO₂R⁹, CO₂R⁹, OC(O)R⁹, C(O)R⁹; C(O)N(R⁹R¹¹); SO₂N(R⁹R¹¹); S(O)N(R⁹R¹¹); N(R⁹)SO₂R¹¹; N(R⁹)SOR¹¹; N(R⁹)SO₂N(R¹⁰R¹¹); N(R⁹R¹¹); N(R⁹)C(O)R¹¹; N(R⁹)C(O)N(R¹¹R¹²); N(R⁹)CO₂R¹¹; OC(O)N(R¹¹R¹²).

In another embodiment a compound of formula (I) is selected where R₁ and R₂ are taken together to form a cyclobutyl ring.

In another embodiment a compound of formula (I) is selected where R₁ and R₂ are taken together to form a 5,5-di substituted spiro[2.3]hexyl ring system.

In another embodiment a compound of formula (I) is selected where R¹⁵ is n-propyl.

In another embodiment a compound of formula (I) is selected where R¹⁵ is isobutyl.

In another embodiment a compound of formula (I) is selected where R¹⁵ is CH₂-cPr.

In another embodiment a compound of formula (I) is selected where R¹⁵ is CH₂-c-Bu.

In another embodiment a compound of formula (I) is selected where R¹⁵ is cyclopentyl.

In certain embodiments of each of Formulas (I), (II) and (III) R¹⁵ is optionally substituted with one or more halo.

In certain embodiments of each of Formulas (I), (II) and (III) R¹⁵ is unsubstituted.

In another embodiment a compound of formula (I) where R³ is phenyl.

In a another embodiment a compound of formula (I) where R³ is phenyl and is optionally substituted with one or more susbstituents independently selected from R⁹, OR⁹, SR⁹, S(O)R⁹, SO₂R⁹, CO₂R⁹, OC(O)R⁹, C(O)R⁹, N(R⁹)SO₂R¹¹ and SO₂N(R⁹R¹¹).

In a further embodiment R³ is phenyl and is optionally substituted with one or more susbstituents independently selected from R⁹, OR⁹, SR⁹, S(O)R⁹, SO₂R⁹, CO₂R⁹, OC(O)R⁹, C(O)R⁹, N(R⁹) SO₂R¹¹ and SO₂N(R⁹R¹¹).

In another embodiment R⁹ is selected the following groups: C₁-C₇-alkyl, C₃-C₇ saturated cycloalkyl, C₃-C₇ partially unsaturated cycloalkyl, saturated 4-8 membered heterocycle, phenyl, (C₁-C₇)-alkoxy and O—(C₂-C₇-alkyl)-O—(C₁-C₄) alkyl each of which is optionally with one or more substituents independently selected from the group F, CI, Br, I, CF₃, CN, OH, oxo, NH₂, NR¹⁰R¹¹.

In another embodiment of R³ is optionally substituted with one or more substituents independently selected from halo, N₃, CN, NO₂, OH, R⁹, OR⁹, SR⁹, S(O)R⁹, SO₂R⁹, CO₂R⁹, OC(O)R⁹, C(O)R⁹; C(O)N(R⁹R¹¹); C(O)NH(R¹¹); N(R⁹R¹¹); NH(R⁹); NH(R¹¹); N(R⁹)C(O)R¹¹; NHC(O)R¹¹; N(R⁹)C(O)N(R¹¹R¹²); NHC(O)N(R¹¹R¹²); N(R⁹)C(O)NH(R¹¹); N(R⁹)C(O)NH(R¹²); N(R⁹)CO₂R¹¹; NHCO₂R¹¹; OC(O)N(R¹¹R¹²); OC(O)NH(R¹¹) or OC(O)NH(R¹²).

In another embodiments R³ is optionally substituted with one or more substituents independently selected from halo, N₃, CN, NO₂, OH, R⁹, OR⁹, SR⁹, S(O)R⁹ or SO₂R⁹.

In certain embodiments of each of Formula (I), (II) and (III) R³ is optionally substituted with one or more substituents independently selected from halo, CN, NO₂, R⁹, OR⁹ or SR⁹.

In another embodiments R³ is optionally substituted with one or more substituents independently selected from CO₂R⁹, OC(O)R⁹, C(O)R⁹; C(O)N(R⁹R¹¹); C(O)NH(R¹¹); N(R⁹R¹¹); NH(R⁹); NH(R¹¹); N(R⁹)C(O)R¹¹; NHC(O)R¹¹; N(R⁹)C(O)N(R¹¹R¹²); NHC(O)N(R¹¹R¹²); N(R⁹)C(O)NH(R¹¹); N(R⁹)C(O)NH(R¹²); N(R⁹)CO₂R¹¹; NHCO₂R¹¹; OC(O)N(R¹¹R¹²); OC(O)NH(R¹¹); OC(O)NH(R¹²).

In another embodiment a compound of formula (I) is selected where R⁴ is selected from C₁-C₆ alkyl, C₁-C₆ alkoxy, —O—(C₂-C₆ alkyl)-OH, —O—(C₂-C₆ alkyl)-O—(C₁-C₆ alkyl), heteroaryl, C₃-C₇ cycloalkyl, heterocycyl, C₁-C₆ alkynyl or —O—(C₁-C₄ alkyl)-Het².

In another embodiment a compound of formula (I) is selected where R⁴ is selected from C₁-C₆ alkyl.

In another embodiment a compound of formula (I) is selected where R⁴ is selected from C₁-C₆ alkoxy.

In another embodiment a compound of formula (I) is selected where R⁴ is —O—(C₂-C₆ alkyl)-O—(C₁-C₆ alkyl).

In another embodiment a compound of formula (I) is selected where R⁴ is heteroaryl.

In another embodiment a compound of formula (I) is selected where R⁴ is C₃-C₇ cycloalkyl.

In another embodiment a compound of formula (I) is selected where R⁴ is heterocycyl.

In another embodiment a compound of formula (I) is selected where R⁴ is C₁-C₆ alkynyl.

In another embodiment a compound of formula (I) is selected where R⁴ is —O—(C₁-C₄ alkyl)-Het².

In another embodiment a compound of formula (I) is selected where R⁴ is trifluoroethoxy.

In another embodiment a compound of formula (I) is selected where R⁴ is —O—(C₁-C₄ alkyl)-Het².

In another embodiment Het² is selected from benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl.

In another embodiment Het² is selected from benzo[c][1,2,5]oxadiazyl or benzo[c][1,2,5]thiadiazolyl.

In another embodiment Het² is benzo[c][1,2,5]oxadiazyl.

In another embodiment Het² is benzo[c][1,2,5]thiadiazolyl.

In another embodiment a compound of formula (I) is selected where X is selected from —C₁-C₆ alkyl, —(C₀-C₆ alkyl)-O—(C₁-C₄ alkyl)-.

In another embodiment a compound of formula (I) is selected where X is selected from —C(O)—, S(O)p—, —C(O)NR⁸—, N(R⁸)—C(O)—, —SO₂N(R⁸)—, —N(R⁸)—SO₂—, —O—C(O)NR⁸—, —N(R⁸)—C(O)—O—, —N(R⁸)—C(O)NR⁸—, —N(R⁸)—C(O)—N(R⁸)—, —C(O)—O—, —O—C(O)—.

In another embodiment a compound of formula (I) is selected where R⁷ is selected from C₁-C₆ alkyl, C₁-C₆ alkoxy, —O—(C₂-C₆ alkyl)-OH, —O—(C₂-C₆ alkyl)-O—(C₁-C₆ alkyl).

In another embodiment a compound of formula (I) is selected where R⁷ is selected from aryl or —(C₁-C₄ alkyl)-aryl.

In another embodiment a compound of formula (I) is selected where R⁷ is selected from heteroaryl or —(C₁-C₄ alkyl)-heteroaryl.

In another embodiment a compound of formula (I) is selected where R⁷ is selected from C₃-C₇ cycloalkyl or —(C₁-C₄ alkyl)-(C₃-C₇)cycloalkyl.

In another embodiment a compound of formula (I) is selected where R⁷ is selected from heterocycyl or —(C₁-C₄ alkyl)-heterocycyl.

In a another embodiment a compound of formula (II) is selected.

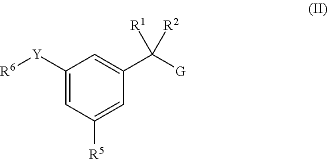

In another embodiment of a compound of formula (II) is selected where G is CO2H.

In another embodiment of a compound of formula (II) is selected where G is a tetrazole.

In another embodiment a compound of formula (II) is selected where G is a carboxylic acid.

In another embodiment a compound of formula (II) is selected where G is a tetrazole.

In another embodiment a compound of formula (II) is selected where R1 and R2 are independently selected from H or R15.

In another embodiment a compound of formula (II) is selected when R1 and R2 groups when taken together to form a mono or bicyclic ring system comprising of 4 to 11 ring atoms selected from C, N, O and S provided that not more than 3 ring atoms in any single ring are other than C and each ring is optionally independently singly or multiply substituted with one or more substituents selected from, halogen, hydroxyl, amino, cyano or a C1-4 alkyl substituent.

In another embodiment a compound of formula (II) is selected where R1 and R2 are taken together to form a 3-7 membered cycloalkyl ring substituted with R25 and R26 where R25 and R26 are attached to the same carbon and taken together to form a second 3-7 membered cycloalkyl ring wherein each cycloalkyl is optionally multiply and independently substituted with halo, hydroxy, cyano, CF₃, C1-C4 alkyl. For example 5,5-spiro[2.3]hexyl system

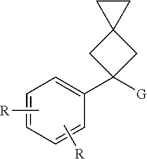

In another embodiment a compound of formula (II) is selected where R15 is C3-C6 alkyl.

In another embodiment a compound of formula (II) is selected where R15 is C1-C6 alkoxy.

In another embodiment a compound of formula (II) is selected where R15 is —O—(C₂-C₆ alkyl)-OH.

In another embodiment a compound of formula (II) is selected where R15 is —O—(C₂-C₆ alkyl)-O—(C1-C6 alkyl).

In another embodiment a compound of formula (II) is selected where R15 is aryl.

In another embodiment a compound of formula (II) is selected where R15 is, —(C1-C4 alkyl)-aryl.

In another embodiment a compound of formula (II) is selected where R15 is heteroaryl.

In another embodiment a compound of formula (II) is selected where R15 is —(C1-C4 alkyl)-heteroaryl.

In another embodiment a compound of formula (II) is selected where R15 is C3-C7 cycloalkyl.

In another embodiment a compound of formula (II) is selected where R15 is —(C1-C4 alkyl)-(C3-C7)cycloalkyl.

In another embodiment a compound of formula (II) is selected where R15 is heterocycyl.

In another embodiment a compound of formula (II) is selected where R15 is —(C1-C4 alkyl)-heterocycyl.

R15 is optionally substituted with one or more substituents independently selected from the group consisting of halo, N3, CN, NO2, oxo, OH, R9, OR9, SR9, S(O)R9, SO2R9, CO2R9, OC(O)R9, C(O)R9; C(O)N(R9R11); SO2N(R9R11); S(O)N(R9R11); N(R9)SO2R11; N(R9)SOR11; N(R9)SO2N(R10R11); N(R9R11); N(R9)C(O)R11; N(R9)C(O)N(R11R12); N(R9)CO2R11; OC(O)N(R11R12).

In another embodiment a compound of formula (I) is selected where R1 and R2 are taken together to form a cyclobutyl ring.

In another embodiment a compound of formula (I) is selected where R1 and R2 are taken together to form a 5,5-di substituted spiro[2.3]hexyl ring system.

In another embodiment a compound of formula (I) is selected where R15 is n-propyl.

In another embodiment a compound of formula (I) is selected where R15 is isobutyl.

In another embodiment a compound of formula (I) is selected where R15 is CH2-cPr.

In another embodiment a compound of formula (I) is selected where R15 is CH2-c-Bu.

In another embodiment a compound of formula (I) is selected where R15 is cyclopentyl.

In another embodiment a compound of formula (II) is selected where R5 is heteroaryl.

In a further embodiment R5 is selected from furyl, thienyl, pyrrolyl, pyrazolyl, imidazolyl, isoxazyl, oxazyl, thiazolyl, isothiazolyl, 1,2,4-oxadiazole, triazozyl, pyridyl, benzo[c][1,2,5]oxadiazolyl, benzo[c][1,2,5]thiadiazolyl, imidazopyridinyl.

In a further embodiment R5 is selected from benzo[c][1,2,5]oxadiazolyl and benzo[c][1,2,5]thiadiazolyl.

In a further embodiment R5 is selected from benzo[c][1,2,5]oxadiazolyl.

In a further embodiment R5 is selected from benzo[c][1,2,5]thiadiazolyl.

In another embodiment R5 is a C3-C7 cycloalkyl.

In another embodiment R5 is a heterocycyl.

In another embodiment a compound of formula (II) is selected where Y is selected from a covalent bond, —O—, N(R8)-.

In another embodiment a compound of formula (II) is selected where Y is selected from —C1-C6 alkyl, O—(C1-C6 alkyl)-, —(C1-C6 alkyl)-O—, —(C1-C6 alkyl)-O—(C1-C6 alkyl)-, —C(O)—, S(O)p-, —O—C(R)(R)—, —C(O)NR8-, C(O)—, —SO2N(R8)-, —N(R8)-SO2-, —O—C(O)NR8-, —N(R)—C(O)—O—, —N(R8)-C(O)NR8-, —N(R8)-C(O)— N(R8)-, —C(O)—O—, —O—C(O)—.

In another embodiment a compound of formula (II) is selected where R6 is selected from C1-C6 alkyl, C1-C6 alkoxy, —O—(C2-C6 alkyl)-OH, —O—(C2-C6 alkyl)-O—(C1-C6 alkyl).

In another embodiment a compound of formula (II) is selected where R6 is selected from aryl or —(C1-C4 alkyl)-aryl.

In another embodiment a compound of formula (II) is selected where R6 is selected from heteroaryl or —(C1-C4 alkyl)-heteroaryl.

In another embodiment a compound of formula (II) is selected where R6 is selected from C3-C7 cycloalkyl or —(C1-C4 alkyl)-(C3-C7)cycloalkyl.

In another embodiment a compound of formula (II) is selected where R6 is selected from heterocycyl or —(C1-C4 alkyl)-heterocycyl.

In another embodiment a compound of formula (III) is selected.

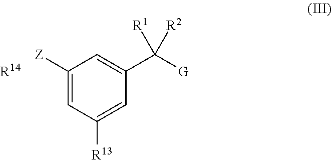

In another embodiment of a compound of formula (III) is selected where G is CO2H.

In another embodiment of a compound of formula (III) is selected where G is a tetrazole.

In another embodiment a compound of formula (III) is selected where G is a carboxylic acid.

In another embodiment a compound of formula (III) is selected where G is a tetrazole.

In another embodiment a compound of formula (III) is selected where R1 and R2 are independently selected from H or R15.

In another embodiment a compound of formula (III) is selected where R1 and R2 are taken together to form a mono or bicyclic ring system having 4 to 11 ring atoms selected from C, N, O and S, provided that not more than 3 ring atoms in any single ring are other than C.

In another embodiment a compound of formula (III) is selected when the R1 and R2 groups when taken together to form a mono or bicyclic ring system comprising of 4 to 11 ring atoms selected from C, N, O and S provided that not more than 3 ring atoms in any single ring are other than C and each ring is optionally independently singly or multiply substituted with one or more substituents selected from, halogen, hydroxyl, amino, cyano or a C1-4 alkyl substituent

In another embodiment a compound of formula (III) is selected where R1 and R2 are taken together to form a 3-7 membered cycloalkyl ring substituted with R25 and R26 where R25 and R26 are attached to the same carbon and taken together to form a second 3-7 membered cycloalkyl ring wherein each cycloalkyl is optionally multiply and independently substituted with halo, hydroxy, cyano, CF3, C1-C4 alkyl.

For example 5,5-spiro[2.3]hexyl system

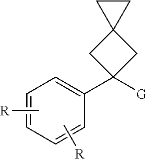

In another embodiment a compound of formula (III) is selected where R15 is C3-C6 alkyl.

In another embodiment a compound of formula (III) is selected where R15 is C1-C6 alkoxy.

In another embodiment a compound of formula (III) is selected where R15 is —O—(C2-C6 alkyl)-OH.

In another embodiment a compound of formula (III) is selected where R15 is —O—(C2-C6 alkyl)-O—(C1-C6 alkyl).

In another embodiment a compound of formula (III) is selected where R15 is aryl.

In another embodiment a compound of formula (III) is selected where R15 is, —(C1-C4 alkyl)-aryl.

In another embodiment a compound of formula (III) is selected where R15 is heteroaryl.

In another embodiment a compound of formula (III) is selected where R15 is —(C1-C4 alkyl)-heteroaryl.

In another embodiment a compound of formula (III) is selected where R15 is C3-C7 cycloalkyl.

In another embodiment a compound of formula (III) is selected where R15 is —(C1-C4 alkyl)-(C3-C7)cycloalkyl.

In another embodiment a compound of formula (III) is selected where R15 is heterocycyl

In another embodiment a compound of formula (III) is selected where R15 is —(C1-C4 alkyl)-heterocycyl.

R15 is optionally substituted with one or more substituents independently selected from the group consisting of halo, N3, CN, NO2, oxo, OH, R9, OR9, SR9, S(O)R9, SO2R9, CO2R9, OC(O)R9, C(O)R9; C(O)N(R9R11); SO2N(R9R11); S(O)N(R9R11); N(R9)SO2R11; N(R9)SOR11; N(R9)SO2N(R10R11); N(R9R11); N(R9)C(O)R11; N(R9)C(O)N(R11R12); N(R9)CO2R11; OC(O)N(R11R12).

In another embodiment a compound of formula (I) is selected where R1 and R2 are taken together to form a cyclobutyl ring.

In another embodiment a compound of formula (I) is selected where R1 and R2 are taken together to form a 5,5-di substituted spiro[2.3]hexyl ring system.

In another embodiment a compound of formula (I) is selected where R15 is n-propyl.

In another embodiment a compound of formula (I) is selected where R15 is isobutyl.

In another embodiment a compound of formula (I) is selected where R15 is CH2-cPr.

In another embodiment a compound of formula (I) is selected where R15 is CH2-c-Bu.

In another embodiment a compound of formula (I) is selected where R15 is cyclopentyl.

In another embodiment a compound of formula (III) is selected where R13 is selected from F, Cl or CF3.

In another embodiment R13 is selected from CN, OCF3, C1-C7 alkyl, C₁₋₇ alkoxy, —O—(C2-C7-alkyl)-O—(C1-4 alkyl).

In another embodiment a compound of formula (III) is selected where R13 is selected from —O—(C2-C7-alkyl)-O—(C1-4 alkyl) and —(C1-C4 alkyl)-(C3-C7)cycloalkyl.

In another embodiment a compound of formula (III) is selected where R13 is —O—(C1-C4 alkyl)-C3-C7 cycloalkyl.

In another embodiment a compound of formula (III) is selected where R13 is selected from F, Cl.

In another embodiment a compound of formula (III) is selected where R13 is CN.

In another embodiment a compound of formula (III) is selected where R13 is OCF3.

In another embodiment a compound of formula (III) is selected where R13 is C1-C7 alkyl or CF3.

In another embodiment a compound of formula (III) is selected where R13 is selected is —O—(C2-C7-alkyl)-O—(C1-4 alkyl).

In another embodiment a compound of formula (III) is selected where R13 is —(C1-C4 alkyl)-(C3-C7)cycloalkyl.

In another embodiment a compound of formula (III) is selected where R13 is selected from —O—(C1-C4 alkyl)-(C3-C7)cycloalkyl.

In another embodiment a compound of formula (III) is selected where Z is selected from —O—, —C1-C6 alkyl, O—(C1-C6 alkyl)-, —(C1-C6 alkyl)-O—, —(C1-C6 alkyl)-O—(C1-C6 alkyl)-,

Where the leftmost radical is attached to R14.

In another embodiment a compound of formula (III) is selected where Z is selected from
—C(O)—, S(O)p-, —O—C(R)(R)—, —C(O)NR8-, N(R8)-C(O)—, —SO2N(R8)-, —N(R8)-SO2-, —O—C(O)NR8-, —N(R)—C(O)—O—, —N(R8)-C(O)NR8-, —N(R8)-C(O)— N(R8)-, —C(O)—O—, —O—C(O)—

where the leftmost radical is attached to R14.

p is 0, 1 or 2.

In another embodiment a compound of formula (III) is selected where R14 is selected from aryl or —(C₁-C₄ alkyl)-aryl.

In another embodiment R¹⁴ is selected from heteroaryl, or —(C₁-C₄ alkyl)-heteroaryl.

In another embodiment R¹⁴ is selected from C₃-C₇ cycloalkyl, or —(C₁-C₄ alkyl)-(C₃-C₇) cycloalkyl.

In another embodiment R¹⁴ is selected from heterocycyl or —(C₁-C₄ alkyl)-heterocycyl.

In another embodiment a compound selected from any of Examples Cpd# 1 to 1929 is selected.

In another embodiment a pharmaceutical composition comprising the compound of any of claims of the previous embodiments and a pharmaceutically acceptable carrier or excipient.

In another embodiment a method for treating a neurodegenerative disorder comprising administering to a patient and effective amount of the pharmaceutical composition of the previous embodiment.

In a further embodiment the method of the previous embodiment wherein the disorder is Alzheimer's disease.

In another embodiment a method of treating a disease characterized by an elevated level of Aβ₄₂ with a compound of any of the previous embodiments In another embodiment a method of lowering Aβ₄₂ in a mammal, which method comprises of administering a therapeutically effective amount of any of the previous embodiments.

### EXAMPLES

A compound of formula (IV)

TABLE 1
(IV)

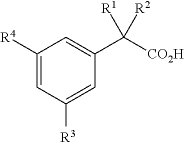

Where

| Cpd # | R¹ | R² | R³ | R⁴ |
|---|---|---|---|---|
| 1 | c-Bu | | 4-CF₃-phenyl- | OMe |
| 2 | CH₂—c-Bu | H | 4-CF₃-phenyl- | OMe |
| 3 | 5,5- | | 4-CF₃-phenyl- | OMe |
| | spiro[2.3]hexane | | | |
| 4 | H | nPr | 4-CF₃-phenyl- | OMe |
| 5 | H | i-Pr | 4-CF₃-phenyl- | OMe |
| 6 | H | nBu | 4-CF₃-phenyl- | OMe |
| 7 | H | i-Bu | 4-CF₃-phenyl- | OMe |
| 8 | H | CH₂—c-Pr | 4-CF₃-phenyl- | OMe |
| 9 | c-Pr | | 4-CF₃-phenyl- | OMe |
| 10 | c-Pentyl | H | 4-CF₃-phenyl- | OMe |
| 11 | c-Bu | | 4-CF₃-phenyl- | OEt |
| 12 | CH₂—c-Bu | H | 4-CF₃-phenyl- | OEt |
| 13 | 5,5- | | 4-CF₃-phenyl- | OEt |
| | spiro[2.3]hexane | | | |
| 14 | H | nPr | 4-CF₃-phenyl- | OEt |
| 15 | H | i-Pr | 4-CF₃-phenyl- | OEt |
| 16 | H | nBu | 4-CF₃-phenyl- | OEt |
| 17 | H | i-Bu | 4-CF₃-phenyl- | OEt |
| 18 | H | CH₂—c-Pr | 4-CF₃-phenyl- | OEt |
| 19 | c-Pr | | 4-CF₃-phenyl- | OEt |
| 20 | c-Pentyl | H | 4-CF₃-phenyl- | OEt |
| 21 | c-Bu | | 4-CF₃-phenyl- | O—nPr |
| 22 | CH₂—c-Bu | H | 4-CF₃-phenyl- | O—nPr |
| 23 | 5,5- | | 4-CF₃-phenyl- | O—nPr |
| | spiro[2.3]hexane | | | |
| 24 | H | nPr | 4-CF₃-phenyl- | O—nPr |
| 25 | H | i-Pr | 4-CF₃-phenyl- | O—nPr |
| 26 | H | nBu | 4-CF₃-phenyl- | O—nPr |
| 27 | H | i-Bu | 4-CF₃-phenyl- | O—nPr |
| 28 | H | CH₂—c-Pr | 4-CF₃-phenyl- | O—nPr |
| 29 | c-Pr | | 4-CF₃-phenyl- | O—nPr |
| 30 | c-Pentyl | H | 4-CF₃-phenyl- | O—nPr |
| 31 | c-Bu | | 4-CF₃-phenyl- | O—iPr |
| 32 | CH₂—c-Bu | H | 4-CF₃-phenyl- | O—iPr |
| 33 | 5,5- | | 4-CF₃-phenyl- | O—iPr |
| | spiro[2.3]hexane | | | |
| 34 | H | nPr | 4-CF₃-phenyl- | O—iPr |
| 35 | H | i-Pr | 4-CF₃-phenyl- | O—iPr |
| 36 | H | nBu | 4-CF₃-phenyl- | O—iPr |
| 37 | H | i-Bu | 4-CF₃-phenyl- | O—iPr |
| 38 | H | CH₂—c-Pr | 4-CF₃-phenyl- | O—iPr |
| 39 | c-Pr | | 4-CF₃-phenyl- | O—iPr |
| 40 | c-Pentyl | H | 4-CF₃-phenyl- | O—iPr |
| 41 | c-Bu | | 4-CF₃-phenyl- | O—CH₂CF₃ |
| 42 | CH₂—c-Bu | H | 4-CF₃-phenyl- | O—CH₂CF₃ |
| 43 | 5,5- | | 4-CF₃-phenyl- | O—CH₂CF₃ |
| | spiro[2.3]hexane | | | |
| 44 | H | nPr | 4-CF₃-phenyl- | O—CH₂CF₃ |
| 45 | H | i-Pr | 4-CF₃-phenyl- | O—CH₂CF₃ |
| 46 | H | nBu | 4-CF₃-phenyl- | O—CH₂CF₃ |
| 47 | H | i-Bu | 4-CF₃-phenyl- | O—CH₂CF₃ |
| 48 | H | CH₂—c-Pr | 4-CF₃-phenyl- | O—CH₂CF₃ |
| 49 | c-Pr | | 4-CF₃-phenyl- | O—CH₂CF₃ |
| 50 | c-Pentyl | H | 4-CF₃-phenyl- | O—CH₂CF₃ |
| 51 | c-Bu | | 4-CF₃-phenyl- | O—CH₂CH₂OMe |
| 52 | CH₂—c-Bu | H | 4-CF₃-phenyl- | O—CH₂CH₂OMe |
| 53 | 5,5- | | 4-CF₃-phenyl- | O—CH₂CH₂OMe |
| | spiro[2.3]hexane | | | |
| 54 | H | nPr | 4-CF₃-phenyl- | O—CH₂CH₂OMe |
| 55 | H | i-Pr | 4-CF₃-phenyl- | O—CH₂CH₂OMe |
| 56 | H | nBu | 4-CF₃-phenyl- | O—CH₂CH₂OMe |
| 57 | H | i-Bu | 4-CF₃-phenyl- | O—CH₂CH₂OMe |
| 58 | H | CH₂—c-Pr | 4-CF₃-phenyl- | O—CH₂CH₂OMe |
| 59 | c-Pr | | 4-CF₃-phenyl- | O—CH₂CH₂OMe |
| 60 | c-Pentyl | H | 4-CF₃-phenyl- | O—CH₂CH₂OMe |
| 61 | c-Bu | | 4-Cl-phenyl- | OMe |
| 62 | CH₂—c-Bu | H | 4-Cl-phenyl- | OMe |
| 63 | 5,5- | | 4-Cl-phenyl- | OMe |
| | spiro[2.3]hexane | | | |
| 64 | H | nPr | 4-Cl-phenyl- | OMe |
| 65 | H | i-Pr | 4-Cl-phenyl- | OMe |
| 66 | H | nBu | 4-Cl-phenyl- | OMe |
| 67 | H | i-Bu | 4-Cl-phenyl- | OMe |
| 68 | H | CH₂—c-Pr | 4-Cl-phenyl- | OMe |
| 69 | c-Pr | | 4-Cl-phenyl- | OMe |
| 70 | c-Pentyl | H | 4-Cl-phenyl- | OMe |
| 71 | c-Bu | | 4-Cl-phenyl- | OEt |
| 72 | CH₂—c-Bu | H | 4-Cl-phenyl- | OEt |
| 73 | 5,5- | | 4-Cl-phenyl- | OEt |
| | spiro[2.3]hexane | | | |
| 74 | H | nPr | 4-Cl-phenyl- | OEt |
| 75 | H | i-Pr | 4-Cl-phenyl- | OEt |
| 76 | H | nBu | 4-Cl-phenyl- | OEt |
| 77 | H | i-Bu | 4-Cl-phenyl- | OEt |
| 78 | H | CH₂—c-Pr | 4-Cl-phenyl- | OEt |
| 79 | c-Pr | | 4-Cl-phenyl- | OEt |
| 80 | c-Pentyl | H | 4-Cl-phenyl- | OEt |
| 81 | c-Bu | | 4-Cl-phenyl- | O—nPr |
| 82 | CH₂—c-Bu | H | 4-Cl-phenyl- | O—nPr |
| 83 | 5,5- | | 4-Cl-phenyl- | O—nPr |
| | spiro[2.3]hexane | | | |
| 84 | H | nPr | 4-Cl-phenyl- | O—nPr |
| 85 | H | i-Pr | 4-Cl-phenyl- | O—nPr |
| 86 | H | nBu | 4-Cl-phenyl- | O—nPr |
| 87 | H | i-Bu | 4-Cl-phenyl- | O—nPr |
| 88 | H | CH₂—c-Pr | 4-Cl-phenyl- | O—nPr |
| 89 | c-Pr | | 4-Cl-phenyl- | O—nPr |
| 90 | c-Pentyl | H | 4-Cl-phenyl- | O—nPr |
| 91 | c-Bu | | 4-Cl-phenyl- | O—iPr |
| 92 | CH₂—c-Bu | H | 4-Cl-phenyl- | O—iPr |
| 93 | 5,5- | | 4-Cl-phenyl- | O—iPr |
| | spiro[2.3]hexane | | | |
| 94 | H | nPr | 4-Cl-phenyl- | O—iPr |
| 95 | H | i-Pr | 4-Cl-phenyl- | O—iPr |
| 96 | H | nBu | 4-Cl-phenyl- | O—iPr |
| 97 | H | i-Bu | 4-Cl-phenyl- | O—iPr |
| 98 | H | CH₂—c-Pr | 4-Cl-phenyl- | O—iPr |
| 99 | c-Pr | | 4-Cl-phenyl- | O—iPr |
| 100 | c-Pentyl | H | 4-Cl-phenyl- | O—iPr |
| 101 | c-Bu | | 4-Cl-phenyl- | O—CH₂CF₃ |
| 102 | CH₂—c-Bu | H | 4-Cl-phenyl- | O—CH₂CF₃ |
| 103 | 5,5- | | 4-Cl-phenyl- | O—CH₂CF₃ |
| | spiro[2.3]hexane | | | |
| 104 | H | nPr | 4-Cl-phenyl- | O—CH₂CF₃ |
| 105 | H | i-Pr | 4-Cl-phenyl- | O—CH₂CF₃ |
| 106 | H | nBu | 4-Cl-phenyl- | O—CH₂CF₃ |
| 107 | H | i-Bu | 4-Cl-phenyl- | O—CH₂CF₃ |
| 108 | H | CH₂—c-Pr | 4-Cl-phenyl- | O—CH₂CF₃ |
| 109 | c-Pr | | 4-Cl-phenyl- | O—CH₂CF₃ |
| 110 | c-Pentyl | H | 4-Cl-phenyl- | O—CH₂CF₃ |
| 111 | c-Bu | | 4-Cl-phenyl- | O—CH₂CH₂OMe |
| 112 | CH₂—c-Bu | H | 4-Cl-phenyl- | O—CH₂CH₂OMe |
| 113 | 5,5- | | 4-Cl-phenyl- | O—CH₂CH₂OMe |
| | spiro[2.3]hexane | | | |
| 114 | H | nPr | 4-Cl-phenyl- | O—CH₂CH₂OMe |
| 115 | H | i-Pr | 4-Cl-phenyl- | O—CH₂CH₂OMe |
| 116 | H | nBu | 4-Cl-phenyl- | O—CH₂CH₂OMe |
| 117 | H | i-Bu | 4-Cl-phenyl- | O—CH₂CH₂OMe |
| 118 | H | CH₂—c-Pr | 4-Cl-phenyl- | O—CH₂CH₂OMe |
| 119 | c-Pr | | 4-Cl-phenyl- | O—CH₂CH₂OMe |
| 120 | c-Pentyl | H | 4-Cl-phenyl- | O—CH₂CH₂OMe |
| 121 | c-Bu | | 4-F-phenyl- | OMe |
| 122 | CH₂—c-Bu | H | 4-F-phenyl- | OMe |
| 123 | 5,5- | | 4-F-phenyl- | OMe |
| | spiro[2.3]hexane | | | |
| 124 | H | nPr | 4-F-phenyl- | OMe |
| 125 | H | i-Pr | 4-F-phenyl- | OMe |
| 126 | H | nBu | 4-F-phenyl- | OMe |
| 127 | H | i-Bu | 4-F-phenyl- | OMe |
| 128 | H | CH₂—c-Pr | 4-F-phenyl- | OMe |
| 129 | c-Pr | | 4-F-phenyl- | OMe |
| 130 | c-Pentyl | H | 4-F-phenyl- | OMe |
| 131 | c-Bu | | 4-F-phenyl- | OEt |
| 132 | CH₂—c-Bu | H | 4-F-phenyl- | OEt |
| 133 | 5,5- | | 4-F-phenyl- | OEt |
| | spiro[2.3]hexane | | | |
| 134 | H | nPr | 4-F-phenyl- | OEt |
| 135 | H | i-Pr | 4-F-phenyl- | OEt |
| 136 | H | nBu | 4-F-phenyl- | OEt |
| 137 | H | i-Bu | 4-F-phenyl- | OEt |
| 138 | H | CH₂—c-Pr | 4-F-phenyl- | OEt |
| 139 | c-Pr | | 4-F-phenyl- | OEt |
| 140 | c-Pentyl | H | 4-F-phenyl- | OEt |
| 141 | c-Bu | | 4-F-phenyl- | O—nPr |
| 142 | CH₂—c-Bu | H | 4-F-phenyl- | O—nPr |
| 143 | 5,5- | | 4-F-phenyl- | O—nPr |
| | spiro[2.3]hexane | | | |
| 144 | H | nPr | 4-F-phenyl- | O—nPr |
| 145 | H | i-Pr | 4-F-phenyl- | O—nPr |
| 146 | H | nBu | 4-F-phenyl- | O—nPr |
| 147 | H | i-Bu | 4-F-phenyl- | O—nPr |
| 148 | H | CH₂—c-Pr | 4-F-phenyl- | O—nPr |
| 149 | c-Pr | | 4-F-phenyl- | O—nPr |
| 150 | c-Pentyl | H | 4-F-phenyl- | O—nPr |
| 151 | c-Bu | | 4-F-phenyl- | O—iPr |
| 152 | CH₂—c-Bu | H | 4-F-phenyl- | O—iPr |
| 153 | 5,5- | | 4-F-phenyl- | O—iPr |
| | spiro[2.3]hexane | | | |
| 154 | H | nPr | 4-F-phenyl- | O—iPr |
| 155 | H | i-Pr | 4-F-phenyl- | O—iPr |
| 156 | H | nBu | 4-F-phenyl- | O—iPr |
| 157 | H | i-Bu | 4-F-phenyl- | O—iPr |
| 158 | H | CH₂—c-Pr | 4-F-phenyl- | O—iPr |
| 158 | c-Pr | | 4-F-phenyl- | O—iPr |
| 160 | c-Pentyl | H | 4-F-phenyl- | O—iPr |
| 161 | c-Bu | | 4-F-phenyl- | O—CH₂CF₃ |
| 162 | CH₂—c-Bu | H | 4-F-phenyl- | O—CH₂CF₃ |
| 163 | 5,5- | | 4-F-phenyl- | O—CH₂CF₃ |
| | spiro[2.3]hexane | | | |
| 164 | H | nPr | 4-F-phenyl- | O—CH₂CF₃ |
| 165 | H | i-Pr | 4-F-phenyl- | O—CH₂CF₃ |
| 166 | H | nBu | 4-F-phenyl- | O—CH₂CF₃ |
| 167 | H | i-Bu | 4-F-phenyl- | O—CH₂CF₃ |
| 168 | H | CH₂—c-Pr | 4-F-phenyl- | O—CH₂CF₃ |
| 169 | c-Pr | | 4-F-phenyl- | O—CH₂CF₃ |
| 170 | c-Pentyl | H | 4-F-phenyl- | O—CH₂CF₃ |
| 171 | c-Bu | | 4-F-phenyl- | O—CH₂CH₂OMe |
| 172 | CH₂—c-Bu | H | 4-F-phenyl- | O—CH₂CH₂OMe |
| 173 | 5,5- | | 4-F-phenyl- | O—CH₂CH₂OMe |
| | spiro[2.3]hexane | | | |
| 174 | H | nPr | 4-F-phenyl- | O—CH₂CH₂OMe |
| 175 | H | i-Pr | 4-F-phenyl- | O—CH₂CH₂OMe |
| 176 | H | nBu | 4-F-phenyl- | O—CH₂CH₂OMe |
| 177 | H | i-Bu | 4-F-phenyl- | O—CH₂CH₂OMe |
| 178 | H | CH₂—c-Pr | 4-F-phenyl- | O—CH₂CH₂OMe |
| 179 | c-Pr | | 4-F-phenyl- | O—CH₂CH₂OMe |
| 180 | c-Pentyl | H | 4-F-phenyl- | O—CH₂CH₂OMe |

A compound of formula (V) where

TABLE 2
(V)

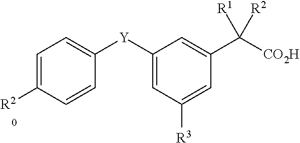

| Cpd # | R¹ | R² | R³ | Y | R²⁰ |
|---|---|---|---|---|---|
| 181 | c-Bu | | 4-CF₃-phenyl- | —C(O)— | F |
| 182 | CH₂—c-Bu | H | 4-CF₃-phenyl- | —C(O)— | F |
| 183 | 5,5- | | 4-CF₃-phenyl- | —C(O)— | F |
| | spiro[2.3] | | | | |
| | hexane | | | | |
| 184 | H | nPr | 4-CF₃-phenyl- | —C(O)— | F |
| 185 | H | i-Pr | 4-CF₃-phenyl- | —C(O)— | F |
| 186 | H | nBu | 4-CF₃-phenyl- | —C(O)— | F |
| 187 | H | i-Bu | 4-CF₃-phenyl- | —C(O)— | F |
| 188 | H | CH₂—c-Pr | 4-CF₃-phenyl- | —C(O)— | F |
| 189 | c-Pr | | 4-CF₃-phenyl- | —C(O)— | F |
| 190 | c-Pentyl | H | 4-CF₃-phenyl- | —C(O)— | F |
| 191 | c-Bu | | 4-CF₃-phenyl- | SO₂ | F |
| 192 | CH₂—c-Bu | H | 4-CF₃-phenyl- | SO₂ | F |
| 193 | 5,5- | | 4-CF₃-phenyl- | SO₂ | F |
| | spiro[2.3] | | | | |
| | hexane | | | | |
| 194 | H | nPr | 4-CF₃-phenyl- | SO₂ | F |
| 195 | H | i-Pr | 4-CF₃-phenyl- | SO₂ | F |
| 196 | H | nBu | 4-CF₃-phenyl- | SO₂ | F |
| 197 | H | i-Bu | 4-CF₃-phenyl- | SO₂ | F |
| 198 | H | CH₂—c-Pr | 4-CF₃-phenyl- | SO₂ | F |
| 199 | c-Pr | | 4-CF₃-phenyl- | SO₂ | F |
| 200 | c-Pentyl | H | 4-CF₃-phenyl- | SO₂ | F |
| 201 | c-Bu | | 4-CF₃-phenyl- | CH₂ | F |
| 202 | CH₂—c-Bu | H | 4-CF₃-phenyl- | CH₂ | F |
| 203 | 5,5- | | 4-CF₃-phenyl- | CH₂ | F |
| | spiro[2.3] | | | | |
| | hexane | | | | |
| 204 | H | nPr | 4-CF₃-phenyl- | CH₂ | F |
| 205 | H | i-Pr | 4-CF₃-phenyl- | CH₂ | F |
| 206 | H | nBu | 4-CF₃-phenyl- | CH₂ | F |
| 207 | H | i-Bu | 4-CF₃-phenyl- | CH₂ | F |
| 208 | H | CH₂—c-Pr | 4-CF₃-phenyl- | CH₂ | F |
| 209 | c-Pr | | 4-CF₃-phenyl- | CH₂ | F |
| 210 | c-Pentyl | H | 4-CF₃-phenyl- | CH₂ | F |
| 211 | c-Bu | | 4-CF₃-phenyl- | —NHSO₂ | F |
| 212 | CH₂—c-Bu | H | 4-CF₃-phenyl- | —NHSO₂ | F |
| 213 | 5,5- | | 4-CF₃-phenyl- | —NHSO₂ | F |
| | spiro[2.3] | | | | |
| | hexane | | | | |
| 214 | H | nPr | 4-CF₃-phenyl- | —NHSO₂ | F |
| 215 | H | i-Pr | 4-CF₃-phenyl- | —NHSO₂ | F |
| 216 | H | nBu | 4-CF₃-phenyl- | —NHSO₂ | F |
| 217 | H | i-Bu | 4-CF₃-phenyl- | —NHSO₂ | F |
| 218 | H | CH₂—c-Pr | 4-CF₃-phenyl- | —NHSO₂ | F |
| 219 | c-Pr | | 4-CF₃-phenyl- | —NHSO₂ | F |
| 220 | c-Pentyl | H | 4-CF₃-phenyl- | —NHSO₂ | F |
| 221 | c-Bu | | 4-CF₃-phenyl- | —SO₂NH— | F |
| 222 | CH₂—c-Bu | H | 4-CF₃-phenyl- | —SO₂NH— | F |
| 223 | 5,5- | | 4-CF₃-phenyl- | —SO₂NH— | F |
| | spiro[2.3] | | | | |
| | hexane | | | | |
| 224 | H | nPr | 4-CF₃-phenyl- | —SO₂NH— | F |
| 225 | H | i-Pr | 4-CF₃-phenyl- | —SO₂NH— | F |
| 226 | H | nBu | 4-CF₃-phenyl- | —SO₂NH— | F |
| 227 | H | i-Bu | 4-CF₃-phenyl- | —SO₂NH— | F |
| 228 | H | CH₂—c-Pr | 4-CF₃-phenyl- | —SO₂NH— | F |
| 229 | c-Pr | | 4-CF₃-phenyl- | —SO₂NH— | F |
| 230 | c-Pentyl | H | 4-CF₃-phenyl- | —SO₂NH— | F |
| 231 | c-Bu | | 4-CF₃-phenyl- | —C(O)— | Cl |
| 232 | CH₂—c-Bu | H | 4-CF₃-phenyl- | —C(O)— | Cl |
| 233 | 5,5- | | 4-CF₃-phenyl- | —C(O)— | Cl |
| | spiro[2.3] | | | | |
| | hexane | | | | |
| 234 | H | nPr | 4-CF₃-phenyl- | —C(O)— | Cl |
| 235 | H | i-Pr | 4-CF₃-phenyl- | —C(O)— | Cl |
| 236 | H | nBu | 4-CF₃-phenyl- | —C(O)— | Cl |
| 237 | H | i-Bu | 4-CF₃-phenyl- | —C(O)— | Cl |
| 238 | H | CH₂—c-Pr | 4-CF₃-phenyl- | —C(O)— | Cl |
| 239 | c-Pr | | 4-CF₃-phenyl- | —C(O)— | Cl |
| 240 | c-Pentyl | H | 4-CF₃-phenyl- | —C(O)— | Cl |
| 241 | | | | | |
| 251 | c-Bu | | 4-CF₃-phenyl- | SO₂ | Cl |
| 252 | CH₂—c-Bu | H | 4-CF₃-phenyl- | SO₂ | Cl |
| 253 | 5,5- | | 4-CF₃-phenyl- | SO₂ | Cl |
| | spiro[2.3] | | | | |
| | hexane | | | | |
| 254 | H | nPr | 4-CF₃-phenyl- | SO₂ | Cl |
| 255 | H | i-Pr | 4-CF₃-phenyl- | SO₂ | Cl |
| 256 | H | nBu | 4-CF₃-phenyl- | SO₂ | Cl |
| 257 | H | i-Bu | 4-CF₃-phenyl- | SO₂ | Cl |
| 258 | H | CH₂—c-Pr | 4-CF₃-phenyl- | SO₂ | Cl |
| 259 | c-Pr | | 4-CF₃-phenyl- | SO₂ | Cl |
| 260 | c-Pentyl | H | 4-CF₃-phenyl- | SO₂ | Cl |
| 261 | c-Bu | | 4-CF₃-phenyl- | CH₂ | Cl |
| 262 | CH₂—c-Bu | H | 4-CF₃-phenyl- | CH₂ | Cl |
| 263 | 5,5- | | 4-CF₃-phenyl- | CH₂ | Cl |
| | spiro[2.3] | | | | |
| | hexane | | | | |
| 264 | H | nPr | 4-CF₃-phenyl- | CH₂ | Cl |
| 265 | H | i-Pr | 4-CF₃-phenyl- | CH₂ | Cl |
| 266 | H | nBu | 4-CF₃-phenyl- | CH₂ | Cl |
| 267 | H | i-Bu | 4-CF₃-phenyl- | CH₂ | Cl |
| 268 | H | CH₂—c-Pr | 4-CF₃-phenyl- | CH₂ | Cl |
| 269 | c-Pr | | 4-CF₃-phenyl- | CH₂ | Cl |
| 270 | c-Pentyl | H | 4-CF₃-phenyl- | CH₂ | Cl |
| 271 | c-Bu | | 4-CF₃-phenyl- | —NHSO₂ | Cl |
| 272 | CH₂—c-Bu | H | 4-CF₃-phenyl- | —NHSO₂ | Cl |
| 273 | 5,5- | | 4-CF₃-phenyl- | —NHSO₂ | Cl |
| | spiro[2.3] | | | | |
| | hexane | | | | |
| 274 | H | nPr | 4-CF₃-phenyl- | —NHSO₂ | Cl |
| 275 | H | i-Pr | 4-CF₃-phenyl- | —NHSO₂ | Cl |
| 276 | H | nBu | 4-CF₃-phenyl- | —NHSO₂ | Cl |
| 277 | H | i-Bu | 4-CF₃-phenyl- | —NHSO₂ | Cl |
| 278 | H | CH₂—c-Pr | 4-CF₃-phenyl- | —NHSO₂ | Cl |
| 279 | c-Pr | | 4-CF₃-phenyl- | —NHSO₂ | Cl |
| 280 | c-Pentyl | H | 4-CF₃-phenyl- | —NHSO₂ | Cl |
| 281 | c-Bu | | 4-CF₃-phenyl- | —SO₂NH— | Cl |
| 282 | CH₂—c-Bu | H | 4-CF₃-phenyl- | —SO₂NH— | Cl |
| 283 | 5,5- | | 4-CF₃-phenyl- | —SO₂NH— | Cl |
| | spiro[2.3] | | | | |
| | hexane | | | | |
| 284 | H | nPr | 4-CF₃-phenyl- | —SO₂NH— | Cl |
| 285 | H | i-Pr | 4-CF₃-phenyl- | —SO₂NH— | Cl |
| 286 | H | nBu | 4-CF₃-phenyl- | —SO₂NH— | Cl |
| 287 | H | i-Bu | 4-CF₃-phenyl- | —SO₂NH— | Cl |
| 288 | H | CH₂—c-Pr | 4-CF₃-phenyl- | —SO₂NH— | Cl |
| 289 | c-Pr | | 4-CF₃-phenyl- | —SO₂NH— | Cl |
| 290 | c-Pentyl | H | 4-CF₃-phenyl- | —SO₂NH— | Cl |
| 291 | c-Bu | | 4-CF₃-phenyl- | —C(O)— | CF₃ |
| 292 | CH₂—c-Bu | H | 4-CF₃-phenyl- | —C(O)— | CF₃ |
| 293 | 5,5- | | 4-CF₃-phenyl- | —C(O)— | CF₃ |
| | spiro[2.3] | | | | |
| | hexane | | | | |
| 294 | H | nPr | 4-CF₃-phenyl- | —C(O)— | CF₃ |
| 295 | H | i-Pr | 4-CF₃-phenyl- | —C(O)— | CF₃ |
| 296 | H | nBu | 4-CF₃-phenyl- | —C(O)— | CF₃ |
| 297 | H | i-Bu | 4-CF₃-phenyl- | —C(O)— | CF₃ |
| 298 | H | CH₂—c-Pr | 4-CF₃-phenyl- | —C(O)— | CF₃ |
| 299 | c-Pr | | 4-CF₃-phenyl- | —C(O)— | CF₃ |
| 300 | c-Pentyl | H | 4-CF₃-phenyl- | —C(O)— | CF₃ |
| 301 | c-Bu | | 4-CF₃-phenyl- | SO₂ | CF₃ |
| 302 | CH₂—c-Bu | H | 4-CF₃-phenyl- | SO₂ | CF₃ |
| 303 | 5,5- | | 4-CF₃-phenyl- | SO₂ | CF₃ |
| | spiro[2.3] | | | | |
| | hexane | | | | |
| 304 | H | nPr | 4-CF₃-phenyl- | SO₂ | CF₃ |
| 305 | H | i-Pr | 4-CF₃-phenyl- | SO₂ | CF₃ |
| 306 | H | nBu | 4-CF₃-phenyl- | SO₂ | CF₃ |
| 307 | H | i-Bu | 4-CF₃-phenyl- | SO₂ | CF₃ |
| 308 | H | CH₂—c-Pr | 4-CF₃-phenyl- | SO₂ | CF₃ |
| 309 | c-Pr | | 4-CF₃-phenyl- | SO₂ | CF₃ |
| 310 | c-Pentyl | H | 4-CF₃-phenyl- | SO₂ | CF₃ |
| 311 | c-Bu | | 4-CF₃-phenyl- | CH₂ | CF₃ |
| 312 | CH₂—c-Bu | H | 4-CF₃-phenyl- | CH₂ | CF₃ |
| 313 | 5,5- | | 4-CF₃-phenyl- | CH₂ | CF₃ |
| | spiro[2.3] | | | | |
| | hexane | | | | |
| 314 | H | nPr | 4-CF₃-phenyl- | CH₂ | CF₃ |
| 315 | H | i-Pr | 4-CF₃-phenyl- | CH₂ | CF₃ |
| 316 | H | nBu | 4-CF₃-phenyl- | CH₂ | CF₃ |
| 317 | H | i-Bu | 4-CF₃-phenyl- | CH₂ | CF₃ |
| 318 | H | CH₂—c-Pr | 4-CF₃-phenyl- | CH₂ | CF₃ |
| 319 | c-Pr | | 4-CF₃-phenyl- | CH₂ | CF₃ |
| 320 | c-Pentyl | H | 4-CF₃-phenyl- | CH₂ | CF₃ |
| 321 | c-Bu | | 4-CF₃-phenyl- | —NHSO₂ | CF₃ |
| 322 | CH₂—c-Bu | H | 4-CF₃-phenyl- | —NHSO₂ | CF₃ |
| 323 | 5,5- | | 4-CF₃-phenyl- | —NHSO₂ | CF₃ |
| | spiro[2.3] | | | | |
| | hexane | | | | |
| 324 | H | nPr | 4-CF₃-phenyl- | —NHSO₂ | CF₃ |
| 325 | H | i-Pr | 4-CF₃-phenyl- | —NHSO₂ | CF₃ |
| 326 | H | nBu | 4-CF₃-phenyl- | —NHSO₂ | CF₃ |
| 327 | H | i-Bu | 4-CF₃-phenyl- | —NHSO₂ | CF₃ |
| 328 | H | CH₂—c-Pr | 4-CF₃-phenyl- | —NHSO₂ | CF₃ |
| 329 | c-Pr | | 4-CF₃-phenyl- | —NHSO₂ | CF₃ |
| 330 | c-Pentyl | H | 4-CF₃-phenyl- | —NHSO₂ | CF₃ |
| 331 | c-Bu | | 4-CF₃-phenyl- | —SO₂NH— | CF₃ |
| 332 | CH₂—c-Bu | H | 4-CF₃-phenyl- | —SO₂NH— | CF₃ |
| 333 | 5,5- | | 4-CF₃-phenyl- | —SO₂NH— | CF₃ |
| | spiro[2.3] | | | | |
| | hexane | | | | |
| 334 | H | nPr | 4-CF₃-phenyl- | —SO₂NH— | CF₃ |
| 335 | H | i-Pr | 4-CF₃-phenyl- | —SO₂NH— | CF₃ |
| 336 | H | nBu | 4-CF₃-phenyl- | —SO₂NH— | CF₃ |
| 337 | H | i-Bu | 4-CF₃-phenyl- | —SO₂NH— | CF₃ |
| 338 | H | CH₂—c-Pr | 4-CF₃-phenyl- | —SO₂NH— | CF₃ |
| 339 | c-Pr | | 4-CF₃-phenyl- | —SO₂NH— | CF₃ |
| 340 | c-Pentyl | H | 4-CF₃-phenyl- | —SO₂NH— | CF₃ |

A compound of formula (VI) where

TABLE 3
(VI)

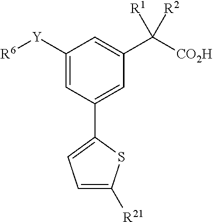

| Cpd# | R¹ | R² | Y | R⁶ | R²¹ |
|---|---|---|---|---|---|
| 341 | c-Bu | | O | —CH₂-cyclopropyl | H |
| 342 | CH₂—c-Bu | H | O | —CH₂-cyclopropyl | H |
| 343 | 5,5- | | O | —CH₂-cyclopropyl | H |
| | spiro[2.3]hexane | | | | |
| 344 | H | nPr | O | —CH₂-cyclopropyl | H |
| 345 | H | i-Pr | O | —CH₂-cyclopropyl | H |
| 346 | H | nBu | O | —CH₂-cyclopropyl | H |
| 347 | H | i-Bu | O | —CH₂-cyclopropyl | H |
| 348 | H | CH₂—c-Pr | O | —CH₂-cyclopropyl | H |
| 349 | c-Pr | | O | —CH₂-cyclopropyl | H |
| 350 | c-Pentyl | H | O | —CH₂-cyclopropyl | H |
| 351 | c-Bu | | O | —CH₂-cyclopropyl | CF₃ |
| 352 | CH₂—c-Bu | H | O | —CH₂-cyclopropyl | CF₃ |
| 353 | 5,5- | | O | —CH₂-cyclopropyl | CF₃ |
| | spiro[2.3]hexane | | | | |
| 354 | H | nPr | O | —CH₂-cyclopropyl | CF₃ |
| 355 | H | i-Pr | O | —CH₂-cyclopropyl | CF₃ |
| 356 | H | nBu | O | —CH₂-cyclopropyl | CF₃ |
| 357 | H | i-Bu | O | —CH₂-cyclopropyl | CF₃ |
| 358 | H | CH₂—c-Pr | O | —CH₂-cyclopropyl | CF₃ |
| 359 | c-Pr | | O | —CH₂-cyclopropyl | CF₃ |
| 360 | c-Pentyl | H | O | —CH₂-cyclopropyl | CF₃ |
| 361 | c-Bu | | O | —CH₂-cyclopropyl | CH₃ |
| 362 | CH₂—c-Bu | H | O | —CH₂-cyclopropyl | CH₃ |
| 363 | 5,5- | | O | —CH₂-cyclopropyl | CH₃ |
| | spiro[2.3]hexane | | | | |
| 364 | H | nPr | O | —CH₂-cyclopropyl | CH₃ |
| 365 | H | i-Pr | O | —CH₂-cyclopropyl | CH₃ |
| 366 | H | nBu | O | —CH₂-cyclopropyl | CH₃ |
| 367 | H | i-Bu | O | —CH₂-cyclopropyl | CH₃ |
| 368 | H | CH₂—c-Pr | O | —CH₂-cyclopropyl | CH₃ |
| 369 | c-Pr | | O | —CH₂-cyclopropyl | CH₃ |
| 370 | c-Pentyl | H | O | —CH₂-cyclopropyl | CH₃ |
| 371 | c-Bu | | O | —CH₂-cyclopropyl | Cl |
| 372 | CH₂—c-Bu | H | O | —CH₂-cyclopropyl | Cl |
| 373 | 5,5- | | O | —CH₂-cyclopropyl | Cl |
| | spiro[2.3]hexane | | | | |
| 374 | H | nPr | O | —CH₂-cyclopropyl | Cl |
| 375 | H | i-Pr | O | —CH₂-cyclopropyl | Cl |
| 376 | H | nBu | O | —CH₂-cyclopropyl | Cl |
| 377 | H | i-Bu | O | —CH₂-cyclopropyl | Cl |
| 378 | H | CH₂—c-Pr | O | —CH₂-cyclopropyl | Cl |
| 379 | c-Pr | | O | —CH₂-cyclopropyl | Cl |
| 380 | c-Pentyl | H | O | —CH₂-cyclopropyl | Cl |
| 381 | c-Bu | | O | —CH₂—p-C₆H₄—F | H |
| 382 | CH₂—c-Bu | H | O | —CH₂—p-C₆H₄—F | H |
| 383 | 5,5- | | O | —CH₂—p-C₆H₄—F | H |
| | spiro[2.3]hexane | | | | |
| 384 | H | nPr | O | —CH₂—p-C₆H₄—F | H |
| 385 | H | i-Pr | O | —CH₂—p-C₆H₄—F | H |
| 386 | H | nBu | O | —CH₂—p-C₆H₄—F | H |
| 387 | H | i-Bu | O | —CH₂—p-C₆H₄—F | H |
| 388 | H | CH₂—c-Pr | O | —CH₂—p-C₆H₄—F | H |
| 389 | c-Pr | | O | —CH₂—p-C₆H₄—F | H |
| 390 | c-Pentyl | H | O | —CH₂—p-C₆H₄—F | H |
| 391 | c-Bu | | O | —CH₂—p-C₆H₄—F | CF₃ |
| 392 | CH₂—c-Bu | H | O | —CH₂—p-C₆H₄—F | CF₃ |
| 393 | 5,5- | | O | —CH₂—p-C₆H₄—F | CF₃ |
| | spiro[2.3]hexane | | | | |
| 394 | H | nPr | O | —CH₂—p-C₆H₄—F | CF₃ |
| 395 | H | i-Pr | O | —CH₂—p-C₆H₄—F | CF₃ |
| 396 | H | nBu | O | —CH₂—p-C₆H₄—F | CF₃ |
| 397 | H | i-Bu | O | —CH₂—p-C₆H₄—F | CF₃ |
| 398 | H | CH₂—c-Pr | O | —CH₂—p-C₆H₄—F | CF₃ |
| 399 | c-Pr | | O | —CH₂—p-C₆H₄—F | CF₃ |
| 400 | c-Pentyl | H | O | —CH₂—p-C₆H₄—F | CF₃ |
| 401 | c-Bu | | O | —CH₂—p-C₆H₄—F | CH₃ |
| 402 | CH₂—c-Bu | H | O | —CH₂—p-C₆H₄—F | CH₃ |
| 403 | 5,5- | | O | —CH₂—p-C₆H₄—F | CH₃ |
| | spiro[2.3]hexane | | | | |
| 404 | H | nPr | O | —CH₂—p-C₆H₄—F | CH₃ |
| 405 | H | i-Pr | O | —CH₂—p-C₆H₄—F | CH₃ |
| 406 | H | nBu | O | —CH₂—p-C₆H₄—F | CH₃ |
| 407 | H | i-Bu | O | —CH₂—p-C₆H₄—F | CH₃ |
| 408 | H | CH₂—c-Pr | O | —CH₂—p-C₆H₄—F | CH₃ |
| 409 | c-Pr | | O | —CH₂—p-C₆H₄—F | CH₃ |
| 410 | c-Pentyl | H | O | —CH₂—p-C₆H₄—F | CH₃ |
| 411 | c-Bu | | O | —CH₂—p-C₆H₄—F | Cl |
| 412 | CH₂—c-Bu | H | O | —CH₂—p-C₆H₄—F | Cl |
| 413 | 5,5- | | O | —CH₂—p-C₆H₄—F | Cl |
| | spiro[2.3]hexane | | | | |
| 414 | H | nPr | O | —CH₂—p-C₆H₄—F | Cl |
| 415 | H | i-Pr | O | —CH₂—p-C₆H₄—F | Cl |
| 416 | H | nBu | O | —CH₂—p-C₆H₄—F | Cl |
| 417 | H | i-Bu | O | —CH₂—p-C₆H₄—F | Cl |
| 418 | H | CH₂—c-Pr | O | —CH₂—p-C₆H₄—F | Cl |
| 419 | c-Pr | | O | —CH₂—p-C₆H₄—F | Cl |
| 420 | c-Pentyl | H | O | —CH₂—p-C₆H₄—F | Cl |
| 421 | c-Bu | | O | —CH₂—p-C₆H₄—Cl | H |
| 422 | CH₂—c-Bu | H | O | —CH₂—p-C₆H₄—Cl | H |
| 423 | 5,5- | | O | —CH₂—p-C₆H₄—Cl | H |
| | spiro[2.3]hexane | | | | |
| 424 | H | nPr | O | —CH₂—p-C₆H₄—Cl | H |
| 425 | H | i-Pr | O | —CH₂—p-C₆H₄—Cl | H |
| 426 | H | nBu | O | —CH₂—p-C₆H₄—Cl | H |
| 427 | H | i-Bu | O | —CH₂—p-C₆H₄—Cl | H |
| 428 | H | CH₂—c-Pr | O | —CH₂—p-C₆H₄—Cl | H |
| 429 | c-Pr | | O | —CH₂—p-C₆H₄—Cl | H |
| 430 | c-Pentyl | H | O | —CH₂—p-C₆H₄—Cl | H |
| 431 | c-Bu | | O | —CH₂—p-C₆H₄—Cl | CF₃ |
| 432 | CH₂—c-Bu | H | O | —CH₂—p-C₆H₄—Cl | CF₃ |
| 433 | 5,5- | | O | —CH₂—p-C₆H₄—Cl | CF₃ |
| | spiro[2.3]hexane | | | | |
| 434 | H | nPr | O | —CH₂—p-C₆H₄—Cl | CF₃ |
| 435 | H | i-Pr | O | —CH₂—p-C₆H₄—Cl | CF₃ |
| 436 | H | nBu | O | —CH₂—p-C₆H₄—Cl | CF₃ |
| 437 | H | i-Bu | O | —CH₂—p-C₆H₄—Cl | CF₃ |
| 438 | H | CH₂—c-Pr | O | —CH₂—p-C₆H₄—Cl | CF₃ |
| 439 | c-Pr | | O | —CH₂—p-C₆H₄—Cl | CF₃ |
| 440 | c-Pentyl | H | O | —CH₂—p-C₆H₄—Cl | CF₃ |
| 441 | c-Bu | | O | —CH₂—p-C₆H₄—Cl | CH₃ |
| 442 | CH₂—c-Bu | H | O | —CH₂—p-C₆H₄—Cl | CH₃ |
| 443 | 5,5- | | O | —CH₂—p-C₆H₄—Cl | CH₃ |
| | spiro[2.3]hexane | | | | |
| 444 | H | nPr | O | —CH₂—p-C₆H₄—Cl | CH₃ |
| 445 | H | i-Pr | O | —CH₂—p-C₆H₄—Cl | CH₃ |
| 446 | H | nBu | O | —CH₂—p-C₆H₄—Cl | CH₃ |
| 447 | H | i-Bu | O | —CH₂—p-C₆H₄—Cl | CH₃ |
| 448 | H | CH₂—c-Pr | O | —CH₂—p-C₆H₄—Cl | CH₃ |
| 449 | c-Pr | | O | —CH₂—p-C₆H₄—Cl | CH₃ |
| 450 | c-Pentyl | H | O | —CH₂—p-C₆H₄—Cl | CH₃ |
| 451 | c-Bu | | O | —CH₂—p-C₆H₄—Cl | Cl |
| 452 | CH₂—c-Bu | H | O | —CH₂—p-C₆H₄—Cl | Cl |
| 453 | 5,5- | | O | —CH₂—p-C₆H₄—Cl | Cl |
| | spiro[2.3]hexane | | | | |
| 454 | H | nPr | O | —CH₂—p-C₆H₄—Cl | Cl |
| 455 | H | i-Pr | O | —CH₂—p-C₆H₄—Cl | Cl |
| 456 | H | nBu | O | —CH₂—p-C₆H₄—Cl | Cl |
| 457 | H | i-Bu | O | —CH₂—p-C₆H₄—Cl | Cl |
| 458 | H | CH₂—c-Pr | O | —CH₂—p-C₆H₄—Cl | Cl |
| 459 | c-Pr | | O | —CH₂—p-C₆H₄—Cl | Cl |
| 460 | c-Pentyl | H | O | —CH₂—p-C₆H₄—Cl | Cl |
| 461 | c-Bu | | O | —CH₂—p-C₆H₄—CF₃ | H |
| 462 | CH₂—c-Bu | H | O | —CH₂—p-C₆H₄—CF₃ | H |
| 463 | 5,5- | | O | —CH₂—p-C₆H₄—CF₃ | H |
| | spiro[2.3]hexane | | | | |
| 464 | H | nPr | O | —CH₂—p-C₆H₄—CF₃ | H |
| 465 | H | i-Pr | O | —CH₂—p-C₆H₄—CF₃ | H |
| 466 | H | nBu | O | —CH₂—p-C₆H₄—CF₃ | H |
| 467 | H | i-Bu | O | —CH₂—p-C₆H₄—CF₃ | H |
| 468 | H | CH₂—c-Pr | O | —CH₂—p-C₆H₄—CF₃ | H |
| 469 | c-Pr | | O | —CH₂—p-C₆H₄—CF₃ | H |
| 470 | c-Pentyl | H | O | —CH₂—p-C₆H₄—CF₃ | H |
| 471 | c-Bu | | O | —CH₂—p-C₆H₄—CF₃ | CF₃ |
| 472 | CH₂—c-Bu | H | O | —CH₂—p-C₆H₄—CF₃ | CF₃ |
| 473 | 5,5- | | O | —CH₂—p-C₆H₄—CF₃ | CF₃ |
| | spiro[2.3]hexane | | | | |
| 474 | H | nPr | O | —CH₂—p-C₆H₄—CF₃ | CF₃ |
| 475 | H | i-Pr | O | —CH₂—p-C₆H₄—CF₃ | CF₃ |
| 476 | H | nBu | O | —CH₂—p-C₆H₄—CF₃ | CF₃ |
| 477 | H | i-Bu | O | —CH₂—p-C₆H₄—CF₃ | CF₃ |
| 478 | H | CH₂—c-Pr | O | —CH₂—p-C₆H₄—CF₃ | CF₃ |
| 479 | c-Pr | | O | —CH₂—p-C₆H₄—CF₃ | CF₃ |
| 480 | c-Pentyl | H | O | —CH₂—p-C₆H₄—CF₃ | CF₃ |
| 481 | c-Bu | | O | —CH₂—p-C₆H₄—CF₃ | CH₃ |
| 482 | CH₂—c-Bu | H | O | —CH₂—p-C₆H₄—CF₃ | CH₃ |
| 483 | 5,5- | | O | —CH₂—p-C₆H₄—CF₃ | CH₃ |
| | spiro[2.3]hexane | | | | |
| 484 | H | nPr | O | —CH₂—p-C₆H₄—CF₃ | CH₃ |
| 485 | H | i-Pr | O | —CH₂—p-C₆H₄—CF₃ | CH₃ |
| 486 | H | nBu | O | —CH₂—p-C₆H₄—CF₃ | CH₃ |
| 487 | H | i-Bu | O | —CH₂—p-C₆H₄—CF₃ | CH₃ |
| 488 | H | CH₂—c-Pr | O | —CH₂—p-C₆H₄—CF₃ | CH₃ |
| 489 | c-Pr | | O | —CH₂—p-C₆H₄—CF₃ | CH₃ |
| 490 | c-Pentyl | H | O | —CH₂—p-C₆H₄—CF₃ | CH₃ |
| 491 | c-Bu | | O | —CH₂—p-C₆H₄—CF₃ | Cl |
| 492 | CH₂—c-Bu | H | O | —CH₂—p-C₆H₄—CF₃ | Cl |
| 493 | 5,5- | | O | —CH₂—p-C₆H₄—CF₃ | Cl |
| | spiro[2.3]hexane | | | | |
| 494 | H | nPr | O | —CH₂—p-C₆H₄—CF₃ | Cl |
| 495 | H | i-Pr | O | —CH₂—p-C₆H₄—CF₃ | Cl |
| 496 | H | nBu | O | —CH₂—p-C₆H₄—CF₃ | Cl |
| 497 | H | i-Bu | O | —CH₂—p-C₆H₄—CF₃ | Cl |
| 498 | H | CH₂—c-Pr | O | —CH₂—p-C₆H₄—CF₃ | Cl |
| 499 | c-Pr | | O | —CH₂—p-C₆H₄—CF₃ | Cl |
| 500 | c-Pentyl | H | O | —CH₂—p-C₆H₄—CF₃ | Cl |
| 501 | c-Bu | | O | Et | H |
| 502 | CH₂—c-Bu | H | O | Et | H |
| 503 | 5,5- | | O | Et | H |
| | spiro[2.3]hexane | | | | |
| 504 | H | nPr | O | Et | H |
| 505 | H | i-Pr | O | Et | H |
| 506 | H | nBu | O | Et | H |
| 507 | H | i-Bu | O | Et | H |
| 508 | H | CH₂—c-Pr | O | Et | H |
| 509 | c-Pr | | O | Et | H |
| 51 | c-Pentyl | H | O | Et | H |
| 511 | c-Bu | | O | Et | CF₃ |
| 512 | CH₂—c-Bu | H | O | Et | CF₃ |
| 513 | 5,5- | | O | Et | CF₃ |
| | spiro[2.3]hexane | | | | |
| 514 | H | nPr | O | Et | CF₃ |
| 515 | H | i-Pr | O | Et | CF₃ |
| 516 | H | nBu | O | Et | CF₃ |
| 517 | H | i-Bu | O | Et | CF₃ |
| 518 | H | CH₂—c-Pr | O | Et | CF₃ |
| 519 | c-Pr | | O | Et | CF₃ |
| 520 | c-Pentyl | H | O | Et | CF₃ |
| 521 | c-Bu | | O | Et | CH₃ |
| 522 | CH₂—c-Bu | H | O | Et | CH₃ |
| 523 | 5,5- | | O | Et | CH₃ |
| | spiro[2.3]hexane | | | | |
| 524 | H | nPr | O | Et | CH₃ |
| 525 | H | i-Pr | O | Et | CH₃ |
| 526 | H | nBu | O | Et | CH₃ |
| 527 | H | i-Bu | O | Et | CH₃ |
| 528 | H | CH₂—c-Pr | O | Et | CH₃ |
| 529 | c-Pr | | O | Et | CH₃ |
| 530 | c-Pentyl | H | O | Et | CH₃ |
| 531 | c-Bu | | O | Et | Cl |
| 532 | CH₂—c-Bu | H | O | Et | Cl |
| 533 | 5,5- | | O | Et | Cl |
| | spiro[2.3]hexane | | | | |
| 534 | H | nPr | O | Et | Cl |
| 535 | H | i-Pr | O | Et | Cl |
| 536 | H | nBu | O | Et | Cl |
| 537 | H | i-Bu | O | Et | Cl |
| 538 | H | CH₂—c-Pr | O | Et | Cl |
| 539 | c-Pr | | O | Et | Cl |
| 540 | c-Pentyl | H | O | Et | Cl |
| 541 | c-Bu | | O | CH₂CF₃ | H |
| 542 | CH₂—c-Bu | H | O | CH₂CF₃ | H |
| 543 | 5,5- | | O | CH₂CF₃ | H |
| | spiro[2.3]hexane | | | | |
| 544 | H | nPr | O | CH₂CF₃ | H |
| 545 | H | i-Pr | O | CH₂CF₃ | H |
| 546 | H | nBu | O | CH₂CF₃ | H |
| 547 | H | i-Bu | O | CH₂CF₃ | H |
| 548 | H | CH₂—c-Pr | O | CH₂CF₃ | H |
| 549 | c-Pr | | O | CH₂CF₃ | H |
| 550 | c-Pentyl | H | O | CH₂CF₃ | H |
| 551 | c-Bu | | O | CH₂CF₃ | CF₃ |
| 552 | CH₂—c-Bu | H | O | CH₂CF₃ | CF₃ |
| 553 | 5,5- | | O | CH₂CF₃ | CF₃ |
| | spiro[2.3]hexane | | | | |
| 554 | H | nPr | O | CH₂CF₃ | CF₃ |
| 555 | H | i-Pr | O | CH₂CF₃ | CF₃ |
| 556 | H | nBu | O | CH₂CF₃ | CF₃ |
| 557 | H | i-Bu | O | CH₂CF₃ | CF₃ |
| 558 | H | CH₂—c-Pr | O | CH₂CF₃ | CF₃ |
| 559 | c-Pr | | O | CH₂CF₃ | CF₃ |
| 560 | c-Pentyl | H | O | CH₂CF₃ | CF₃ |
| 561 | c-Bu | | O | CH₂CF₃ | CH₃ |
| 562 | CH₂—c-Bu | H | O | CH₂CF₃ | CH₃ |
| 563 | 5,5- | | O | CH₂CF₃ | CH₃ |
| | spiro[2.3]hexane | | | | |
| 564 | H | nPr | O | CH₂CF₃ | CH₃ |
| 565 | H | i-Pr | O | CH₂CF₃ | CH₃ |
| 566 | H | nBu | O | CH₂CF₃ | CH₃ |
| 567 | H | i-Bu | O | CH₂CF₃ | CH₃ |
| 568 | H | CH₂—c-Pr | O | CH₂CF₃ | CH₃ |
| 569 | c-Pr | | O | CH₂CF₃ | CH₃ |
| 570 | c-Pentyl | H | O | CH₂CF₃ | CH₃ |
| 571 | c-Bu | | O | CH₂CF₃ | Cl |
| 572 | CH₂—c-Bu | H | O | CH₂CF₃ | Cl |
| 573 | 5,5- | | O | CH₂CF₃ | Cl |
| | spiro[2.3]hexane | | | | |
| 574 | H | nPr | O | CH₂CF₃ | Cl |
| 575 | H | i-Pr | O | CH₂CF₃ | Cl |
| 576 | H | nBu | O | CH₂CF₃ | Cl |
| 577 | H | i-Bu | O | CH₂CF₃ | Cl |
| 578 | H | CH₂—c-Pr | O | CH₂CF₃ | Cl |
| 579 | c-Pr | | O | CH₂CF₃ | Cl |
| 580 | c-Pentyl | H | O | CH₂CF₃ | Cl |
| 581 | c-Bu | | O | CH₂CH₂OMe | H |
| 582 | CH₂—c-Bu | H | O | CH₂CH₂OMe | H |
| 583 | 5,5- | | O | CH₂CH₂OMe | H |
| | spiro[2.3]hexane | | | | |
| 584 | H | nPr | O | CH₂CH₂OMe | H |
| 585 | H | i-Pr | O | CH₂CH₂OMe | H |
| 586 | H | nBu | O | CH₂CH₂OMe | H |
| 587 | H | i-Bu | O | CH₂CH₂OMe | H |
| 588 | H | CH₂—c-Pr | O | CH₂CH₂OMe | H |
| 589 | c-Pr | | O | CH₂CH₂OMe | H |
| 590 | c-Pentyl | H | O | CH₂CH₂OMe | H |
| 591 | c-Bu | | O | CH₂CH₂OMe | CF₃ |
| 592 | CH₂—c-Bu | H | O | CH₂CH₂OMe | CF₃ |
| 593 | 5,5- | | O | CH₂CH₂OMe | CF₃ |
| | spiro[2.3]hexane | | | | |
| 594 | H | nPr | O | CH₂CH₂OMe | CF₃ |
| 595 | H | i-Pr | O | CH₂CH₂OMe | CF₃ |
| 596 | H | nBu | O | CH₂CH₂OMe | CF₃ |
| 597 | H | i-Bu | O | CH₂CH₂OMe | CF₃ |
| 598 | H | CH₂—c-Pr | O | CH₂CH₂OMe | CF₃ |
| 599 | c-Pr | | O | CH₂CH₂OMe | CF₃ |
| 600 | c-Pentyl | H | O | CH₂CH₂OMe | CF₃ |
| 601 | c-Bu | | O | CH₂CH₂OMe | CH₃ |
| 602 | CH₂—c-Bu | H | O | CH₂CH₂OMe | CH₃ |
| 603 | 5,5- | | O | CH₂CH₂OMe | CH₃ |
| | spiro[2.3]hexane | | | | |
| 604 | H | nPr | O | CH₂CH₂OMe | CH₃ |
| 605 | H | i-Pr | O | CH₂CH₂OMe | CH₃ |
| 606 | H | nBu | O | CH₂CH₂OMe | CH₃ |
| 607 | H | i-Bu | O | CH₂CH₂OMe | CH₃ |
| 608 | H | CH₂—c-Pr | O | CH₂CH₂OMe | CH₃ |
| 609 | c-Pr | | O | CH₂CH₂OMe | CH₃ |
| 610 | c-Pentyl | H | O | CH₂CH₂OMe | CH₃ |
| 611 | c-Bu | | O | CH₂CH₂OMe | Cl |
| 612 | CH₂—c-Bu | H | O | CH₂CH₂OMe | Cl |
| 613 | 5,5- | | O | CH₂CH₂OMe | Cl |
| | spiro[2.3]hexane | | | | |
| 614 | H | nPr | O | CH₂CH₂OMe | Cl |
| 615 | H | i-Pr | O | CH₂CH₂OMe | Cl |
| 616 | H | nBu | O | CH₂CH₂OMe | Cl |
| 617 | H | i-Bu | O | CH₂CH₂OMe | Cl |
| 618 | H | CH₂—c-Pr | O | CH₂CH₂OMe | Cl |
| 619 | c-Pr | | O | CH₂CH₂OMe | Cl |
| 620 | c-Pentyl | H | O | CH₂CH₂OMe | Cl |
| 621 | c-Bu | | — | p-C₆H₄—F | H |
| 622 | CH₂—c-Bu | H | — | p-C₆H₄—F | H |
| 623 | 5,5- | | — | p-C₆H₄—F | H |
| | spiro[2.3]hexane | | | | |
| 624 | H | nPr | — | p-C₆H₄—F | H |
| 625 | H | i-Pr | — | p-C₆H₄—F | H |
| 626 | H | nBu | — | p-C₆H₄—F | H |
| 627 | H | i-Bu | — | p-C₆H₄—F | H |
| 628 | H | CH₂—c-Pr | — | p-C₆H₄—F | H |
| 629 | c-Pr | | — | p-C₆H₄—F | H |
| 630 | c-Pentyl | H | — | p-C₆H₄—F | H |
| 631 | c-Bu | | — | p-C₆H₄—F | CF₃ |
| 632 | CH₂—c-Bu | H | — | p-C₆H₄—F | CF₃ |
| 633 | 5,5- | | — | p-C₆H₄—F | CF₃ |
| | spiro[2.3]hexane | | | | |
| 634 | H | nPr | — | p-C₆H₄—F | CF₃ |
| 635 | H | i-Pr | — | p-C₆H₄—F | CF₃ |
| 636 | H | nBu | — | p-C₆H₄—F | CF₃ |
| 637 | H | i-Bu | — | p-C₆H₄—F | CF₃ |
| 638 | H | CH₂—c-Pr | — | p-C₆H₄—F | CF₃ |
| 639 | c-Pr | | — | p-C₆H₄—F | CF₃ |
| 640 | c-Pentyl | H | — | p-C₆H₄—F | CF₃ |
| 641 | c-Bu | | — | p-C₆H₄—F | CH₃ |
| 642 | CH₂—c-Bu | H | — | p-C₆H₄—F | CH₃ |
| 643 | 5,5- | | — | p-C₆H₄—F | CH₃ |
| | spiro[2.3]hexane | | | | |
| 644 | H | nPr | — | p-C₆H₄—F | CH₃ |
| 645 | H | i-Pr | — | p-C₆H₄—F | CH₃ |
| 646 | H | nBu | — | p-C₆H₄—F | CH₃ |
| 647 | H | i-Bu | — | p-C₆H₄—F | CH₃ |
| 648 | H | CH₂—c-Pr | — | p-C₆H₄—F | CH₃ |
| 649 | c-Pr | | — | p-C₆H₄—F | CH₃ |
| 650 | c-Pentyl | H | — | p-C₆H₄—F | CH₃ |
| 651 | c-Bu | | — | p-C₆H₄—F | Cl |
| 652 | CH₂—c-Bu | H | — | p-C₆H₄—F | Cl |
| 653 | 5,5- | | — | p-C₆H₄—F | Cl |
| | spiro[2.3]hexane | | | | |
| 654 | H | nPr | — | p-C₆H₄—F | Cl |
| 655 | H | i-Pr | — | p-C₆H₄—F | Cl |
| 656 | H | nBu | — | p-C₆H₄—F | Cl |
| 657 | H | i-Bu | — | p-C₆H₄—F | Cl |
| 658 | H | CH₂—c-Pr | — | p-C₆H₄—F | Cl |
| 659 | c-Pr | | — | p-C₆H₄—F | Cl |
| 660 | c-Pentyl | H | — | p-C₆H₄—F | Cl |
| 661 | c-Bu | | — | p-C₆H₄—Cl | H |
| 662 | CH₂—c-Bu | H | — | p-C₆H₄—Cl | H |
| 663 | 5,5- | | — | p-C₆H₄—Cl | H |
| | spiro[2.3]hexane | | | | |
| 664 | H | nPr | — | p-C₆H₄—Cl | H |
| 665 | H | i-Pr | — | p-C₆H₄—Cl | H |
| 666 | H | nBu | — | p-C₆H₄—Cl | H |
| 667 | H | i-Bu | — | p-C₆H₄—Cl | H |
| 668 | H | CH₂—c-Pr | — | p-C₆H₄—Cl | H |
| 669 | c-Pr | | — | p-C₆H₄—Cl | H |
| 670 | c-Pentyl | H | — | p-C₆H₄—Cl | H |
| 671 | c-Bu | | — | p-C₆H₄—Cl | CF₃ |
| 672 | CH₂—c-Bu | H | — | p-C₆H₄—Cl | CF₃ |
| 673 | 5,5- | | — | p-C₆H₄—Cl | CF₃ |
| | spiro[2.3]hexane | | | | |
| 674 | H | nPr | — | p-C₆H₄—Cl | CF₃ |
| 675 | H | i-Pr | — | p-C₆H₄—Cl | CF₃ |
| 676 | H | nBu | — | p-C₆H₄—Cl | CF₃ |
| 677 | H | i-Bu | — | p-C₆H₄—Cl | CF₃ |
| 678 | H | CH₂—c-Pr | — | p-C₆H₄—Cl | CF₃ |
| 679 | c-Pr | | — | p-C₆H₄—Cl | CF₃ |
| 680 | c-Pentyl | H | — | p-C₆H₄—Cl | CF₃ |
| 681 | c-Bu | | — | p-C₆H₄—Cl | CH₃ |
| 682 | CH₂—c-Bu | H | — | p-C₆H₄—Cl | CH₃ |
| 683 | 5,5- | | — | p-C₆H₄—Cl | CH₃ |
| | spiro[2.3]hexane | | | | |
| 684 | H | nPr | — | p-C₆H₄—Cl | CH₃ |
| 685 | H | i-Pr | — | p-C₆H₄—Cl | CH₃ |
| 686 | H | nBu | — | p-C₆H₄—Cl | CH₃ |
| 687 | H | i-Bu | — | p-C₆H₄—Cl | CH₃ |
| 688 | H | CH₂—c-Pr | — | p-C₆H₄—Cl | CH₃ |
| 689 | c-Pr | | — | p-C₆H₄—Cl | CH₃ |
| 690 | c-Pentyl | H | — | p-C₆H₄—Cl | CH₃ |
| 691 | c-Bu | | — | p-C₆H₄—Cl | Cl |
| 692 | CH₂—c-Bu | H | — | p-C₆H₄—Cl | Cl |
| 693 | 5,5- | | — | p-C₆H₄—Cl | Cl |
| | spiro[2.3]hexane | | | | |
| 694 | H | nPr | — | p-C₆H₄—Cl | Cl |
| 695 | H | i-Pr | — | p-C₆H₄—Cl | Cl |
| 696 | H | nBu | — | p-C₆H₄—Cl | Cl |
| 697 | H | i-Bu | — | p-C₆H₄—Cl | Cl |
| 698 | H | CH₂—c-Pr | — | p-C₆H₄—Cl | Cl |
| 699 | c-Pr | | — | p-C₆H₄—Cl | Cl |
| 700 | c-Pentyl | H | — | p-C₆H₄—Cl | Cl |
| 701 | c-Bu | | — | p-C₆H₄—CF₃ | H |
| 702 | CH₂—c-Bu | H | — | p-C₆H₄—CF₃ | H |
| 703 | 5,5- | | — | p-C₆H₄—CF₃ | H |
| | spiro[2.3]hexane | | | | |
| 704 | H | nPr | — | p-C₆H₄—CF₃ | H |
| 705 | H | i-Pr | — | p-C₆H₄—CF₃ | H |
| 706 | H | nBu | — | p-C₆H₄—CF₃ | H |
| 707 | H | i-Bu | — | p-C₆H₄—CF₃ | H |
| 708 | H | CH₂—c-Pr | — | p-C₆H₄—CF₃ | H |
| 709 | c-Pr | | — | p-C₆H₄—CF₃ | H |
| 710 | c-Pentyl | H | — | p-C₆H₄—CF₃ | H |
| 711 | c-Bu | | — | p-C₆H₄—CF₃ | CF₃ |
| 712 | CH₂—c-Bu | H | — | p-C₆H₄—CF₃ | CF₃ |
| 713 | 5,5- | | — | p-C₆H₄—CF₃ | CF₃ |
| | spiro[2.3]hexane | | | | |
| 714 | H | nPr | — | p-C₆H₄—CF₃ | CF₃ |
| 715 | H | i-Pr | — | p-C₆H₄—CF₃ | CF₃ |
| 716 | H | nBu | — | p-C₆H₄—CF₃ | CF₃ |
| 717 | H | i-Bu | — | p-C₆H₄—CF₃ | CF₃ |
| 718 | H | CH₂—c-Pr | — | p-C₆H₄—CF₃ | CF₃ |
| 719 | c-Pr | | — | p-C₆H₄—CF₃ | CF₃ |
| 720 | c-Pentyl | H | — | p-C₆H₄—CF₃ | CF₃ |
| 721 | c-Bu | | — | p-C₆H₄—CF₃ | CH₃ |
| 722 | CH₂—c-Bu | H | — | p-C₆H₄—CF₃ | CH₃ |
| 723 | 5,5- | | — | p-C₆H₄—CF₃ | CH₃ |
| | spiro[2.3]hexane | | | | |
| 724 | H | nPr | — | p-C₆H₄—CF₃ | CH₃ |
| 725 | H | i-Pr | — | p-C₆H₄—CF₃ | CH₃ |
| 726 | H | nBu | — | p-C₆H₄—CF₃ | CH₃ |
| 727 | H | i-Bu | — | p-C₆H₄—CF₃ | CH₃ |
| 728 | H | CH₂—c-Pr | — | p-C₆H₄—CF₃ | CH₃ |
| 729 | c-Pr | | — | p-C₆H₄—CF₃ | CH₃ |
| 730 | c-Pentyl | H | — | p-C₆H₄—CF₃ | CH₃ |
| 731 | c-Bu | | — | p-C₆H₄—CF₃ | Cl |
| 732 | CH₂—c-Bu | H | — | p-C₆H₄—CF₃ | Cl |
| 733 | 5,5- | | — | p-C₆H₄—CF₃ | Cl |
| | spiro[2.3]hexane | | | | |
| 734 | H | nPr | — | p-C₆H₄—CF₃ | Cl |
| 735 | H | i-Pr | — | p-C₆H₄—CF₃ | Cl |
| 736 | H | nBu | — | p-C₆H₄—CF₃ | Cl |
| 737 | H | i-Bu | — | p-C₆H₄—CF₃ | Cl |
| 738 | H | CH₂—c-Pr | — | p-C₆H₄—CF₃ | Cl |
| 739 | c-Pr | | — | p-C₆H₄—CF₃ | Cl |
| 740 | c-Pentyl | H | — | p-C₆H₄—CF₃ | Cl |

A compound of formula (VII) where

TABLE 4
(VII)

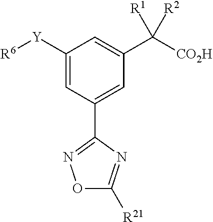

| Cpd# | R¹ | R² | Y | R⁶ | R²² |
|---|---|---|---|---|---|
| 741 | c-Bu | | O | —CH₂-cyclopropyl | H |
| 742 | CH₂—c-Bu | H | O | —CH₂-cyclopropyl | H |
| 743 | 5,5- | | O | —CH₂-cyclopropyl | H |
| | spiro[2.3]hexane | | | | |
| 744 | H | nPr | O | —CH₂-cyclopropyl | H |
| 745 | H | i-Pr | O | —CH₂-cyclopropyl | H |
| 746 | H | nBu | O | —CH₂-cyclopropyl | H |
| 747 | H | i-Bu | O | —CH₂-cyclopropyl | H |
| 748 | H | CH₂—c-Pr | O | —CH₂-cyclopropyl | H |
| 749 | c-Pr | | O | —CH₂-cyclopropyl | H |
| 750 | c-Pentyl | H | O | —CH₂-cyclopropyl | H |
| 751 | c-Bu | | O | —CH₂-cyclopropyl | CF₃ |
| 752 | CH₂—c-Bu | H | O | —CH₂-cyclopropyl | CF₃ |
| 753 | 5,5- | | O | —CH₂-cyclopropyl | CF₃ |
| | spiro[2.3]hexane | | | | |
| 754 | H | nPr | O | —CH₂-cyclopropyl | CF₃ |
| 755 | H | i-Pr | O | —CH₂-cyclopropyl | CF₃ |
| 756 | H | nBu | O | —CH₂-cyclopropyl | CF₃ |
| 757 | H | i-Bu | O | —CH₂-cyclopropyl | CF₃ |
| 758 | H | CH₂—c-Pr | O | —CH₂-cyclopropyl | CF₃ |
| 759 | c-Pr | | O | —CH₂-cyclopropyl | CF₃ |
| 760 | c-Pentyl | H | O | —CH₂-cyclopropyl | CF₃ |
| 761 | c-Bu | | O | —CH₂-cyclopropyl | CH₃ |
| 762 | CH₂—c-Bu | H | O | —CH₂-cyclopropyl | CH₃ |
| 763 | 5,5- | | O | —CH₂-cyclopropyl | CH₃ |
| | spiro[2.3]hexane | | | | |
| 764 | H | nPr | O | —CH₂-cyclopropyl | CH₃ |
| 765 | H | i-Pr | O | —CH₂-cyclopropyl | CH₃ |
| 766 | H | nBu | O | —CH₂-cyclopropyl | CH₃ |
| 767 | H | i-Bu | O | —CH₂-cyclopropyl | CH₃ |
| 768 | H | CH₂—c-Pr | O | —CH₂-cyclopropyl | CH₃ |
| 769 | c-Pr | | O | —CH₂-cyclopropyl | CH₃ |
| 770 | c-Pentyl | H | O | —CH₂-cyclopropyl | CH₃ |
| 771 | c-Bu | | O | —CH₂—p-C₆H₄—F | H |
| 772 | CH₂—c-Bu | H | O | —CH₂—p-C₆H₄—F | H |
| 773 | 5,5- | | O | —CH₂—p-C₆H₄—F | H |
| | spiro[2.3]hexane | | | | |
| 774 | H | nPr | O | —CH₂—p-C₆H₄—F | H |
| 775 | H | i-Pr | O | —CH₂—p-C₆H₄—F | H |
| 776 | H | nBu | O | —CH₂—p-C₆H₄—F | H |
| 777 | H | i-Bu | O | —CH₂—p-C₆H₄—F | H |
| 778 | H | CH₂—c-Pr | O | —CH₂—p-C₆H₄—F | H |
| 779 | c-Pr | | O | —CH₂—p-C₆H₄—F | H |
| 780 | c-Pentyl | H | O | —CH₂—p-C₆H₄—F | H |
| 781 | c-Bu | | O | —CH₂—p-C₆H₄—F | CF₃ |
| 782 | CH₂—c-Bu | H | O | —CH₂—p-C₆H₄—F | CF₃ |
| 783 | 5,5- | | O | —CH₂—p-C₆H₄—F | CF₃ |
| | spiro[2.3]hexane | | | | |
| 784 | H | nPr | O | —CH₂—p-C₆H₄—F | CF₃ |
| 785 | H | i-Pr | O | —CH₂—p-C₆H₄—F | CF₃ |
| 786 | H | nBu | O | —CH₂—p-C₆H₄—F | CF₃ |
| 787 | H | i-Bu | O | —CH₂—p-C₆H₄—F | CF₃ |
| 788 | H | CH₂—c-Pr | O | —CH₂—p-C₆H₄—F | CF₃ |
| 789 | c-Pr | | O | —CH₂—p-C₆H₄—F | CF₃ |
| 790 | c-Pentyl | H | O | —CH₂—p-C₆H₄—F | CF₃ |
| 791 | c-Bu | | O | —CH₂—p-C₆H₄—F | CH₃ |
| 792 | CH₂—c-Bu | H | O | —CH₂—p-C₆H₄—F | CH₃ |
| 793 | 5,5- | | O | —CH₂—p-C₆H₄—F | CH₃ |
| | spiro[2.3]hexane | | | | |
| 794 | H | nPr | O | —CH₂—p-C₆H₄—F | CH₃ |
| 795 | H | i-Pr | O | —CH₂—p-C₆H₄—F | CH₃ |
| 796 | H | nBu | O | —CH₂—p-C₆H₄—F | CH₃ |
| 797 | H | i-Bu | O | —CH₂—p-C₆H₄—F | CH₃ |
| 798 | H | CH₂—c-Pr | O | —CH₂—p-C₆H₄—F | CH₃ |
| 799 | c-Pr | | O | —CH₂—p-C₆H₄—F | CH₃ |
| 800 | c-Pentyl | H | O | —CH₂—p-C₆H₄—F | CH₃ |
| 801 | c-Bu | | O | —CH₂—p-C₆H₄—Cl | H |
| 802 | CH₂—c-Bu | H | O | —CH₂—p-C₆H₄—Cl | H |
| 803 | 5,5- | | O | —CH₂—p-C₆H₄—Cl | H |
| | spiro[2.3]hexane | | | | |
| 804 | H | nPr | O | —CH₂—p-C₆H₄—Cl | H |
| 805 | H | i-Pr | O | —CH₂—p-C₆H₄—Cl | H |
| 806 | H | nBu | O | —CH₂—p-C₆H₄—Cl | H |
| 807 | H | i-Bu | O | —CH₂—p-C₆H₄—Cl | H |
| 808 | H | CH₂—c-Pr | O | —CH₂—p-C₆H₄—Cl | H |
| 809 | c-Pr | | O | —CH₂—p-C₆H₄—Cl | H |
| 810 | c-Pentyl | H | O | —CH₂—p-C₆H₄—Cl | H |
| 811 | c-Bu | | O | —CH₂—p-C₆H₄—C1 | CF₃ |
| 812 | CH₂—c-Bu | H | O | —CH₂—p-C₆H₄—Cl | CF₃ |
| 813 | 5,5- | | O | —CH₂—p-C₆H₄—Cl | CF₃ |
| | spiro[2.3]hexane | | | | |
| 814 | H | nPr | O | —CH₂—p-C₆H₄—Cl | CF₃ |
| 815 | H | i-Pr | O | —CH₂—p-C₆H₄—Cl | CF₃ |
| 816 | H | nBu | O | —CH₂—p-C₆H₄—Cl | CF₃ |
| 817 | H | i-Bu | O | —CH₂—p-C₆H₄—Cl | CF₃ |
| 818 | H | CH₂—c-Pr | O | —CH₂—p-C₆H₄—Cl | CF₃ |
| 819 | c-Pr | | O | —CH₂—p-C₆H₄—Cl | CF₃ |
| 820 | c-Pentyl | H | O | —CH₂—p-C₆H₄—Cl | CF₃ |
| 821 | c-Bu | | O | —CH₂—p-C₆H₄—Cl | CH₃ |
| 822 | CH₂—c-Bu | H | O | —CH₂—p-C₆H₄—Cl | CH₃ |
| 823 | 5,5- | | O | —CH₂—p-C₆H₄—Cl | CH₃ |
| | spiro[2.3]hexane | | | | |
| 824 | H | nPr | O | —CH₂—p-C₆H₄—Cl | CH₃ |
| 825 | H | i-Pr | O | —CH₂—p-C₆H₄—Cl | CH₃ |
| 826 | H | nBu | O | —CH₂—p-C₆H₄—Cl | CH₃ |
| 827 | H | i-Bu | O | —CH₂—p-C₆H₄—Cl | CH₃ |
| 828 | H | CH₂—c-Pr | O | —CH₂—p-C₆H₄—Cl | CH₃ |
| 829 | c-Pr | | O | —CH₂—p-C₆H₄—Cl | CH₃ |
| 830 | c-Pentyl | H | O | —CH₂—p-C₆H₄—Cl | CH₃ |
| 831 | c-Bu | | O | —CH₂—p-C₆H₄—CF₃ | H |
| 832 | CH₂—c-Bu | H | O | —CH₂—p-C₆H₄—CF₃ | H |
| 833 | 5,5- | | O | —CH₂—p-C₆H₄—CF₃ | H |
| | spiro[2.3]hexane | | | | |
| 834 | H | nPr | O | —CH₂—p-C₆H₄—CF₃ | H |
| 835 | H | i-Pr | O | —CH₂—p-C₆H₄—CF₃ | H |
| 836 | H | nBu | O | —CH₂—p-C₆H₄—CF₃ | H |
| 837 | H | i-Bu | O | —CH₂—p-C₆H₄—CF₃ | H |
| 838 | H | CH₂—c-Pr | O | —CH₂—p-C₆H₄—CF₃ | H |
| 839 | c-Pr | | O | —CH₂—p-C₆H₄—CF₃ | H |
| 840 | c-Pentyl | H | O | —CH₂—p-C₆H₄—CF₃ | H |
| 841 | c-Bu | | O | —CH₂—p-C₆H₄—CF₃ | CF₃ |
| 842 | CH₂—c-Bu | H | O | —CH₂—p-C₆H₄—CF₃ | CF₃ |
| 843 | 5,5- | | O | —CH₂—p-C₆H₄—CF₃ | CF₃ |
| | spiro[2.3]hexane | | | | |
| 844 | H | nPr | O | —CH₂—p-C₆H₄—CF₃ | CF₃ |
| 845 | H | i-Pr | O | —CH₂—p-C₆H₄—CF₃ | CF₃ |
| 846 | H | nBu | O | —CH₂—p-C₆H₄—CF₃ | CF₃ |
| 847 | H | i-Bu | O | —CH₂—p-C₆H₄—CF₃ | CF₃ |
| 848 | H | CH₂—c-Pr | O | —CH₂—p-C₆H₄—CF₃ | CF₃ |
| 849 | c-Pr | | O | —CH₂—p-C₆H₄—CF₃ | CF₃ |
| 850 | c-Pentyl | H | O | —CH₂—p-C₆H₄—CF₃ | CF₃ |
| 851 | c-Bu | | O | —CH₂—p-C₆H₄—CF₃ | CH₃ |
| 852 | CH₂—c-Bu | H | O | —CH₂—p-C₆H₄—CF₃ | CH₃ |
| 853 | 5,5- | | O | —CH₂—p-C₆H₄—CF₃ | CH₃ |
| | spiro[2.3]hexane | | | | |
| 854 | H | nPr | O | —CH₂—p-C₆H₄—CF₃ | CH₃ |
| 855 | H | i-Pr | O | —CH₂—p-C₆H₄—CF₃ | CH₃ |
| 856 | H | nBu | O | —CH₂—p-C₆H₄—CF₃ | CH₃ |
| 857 | H | i-Bu | O | —CH₂—p-C₆H₄—CF₃ | CH₃ |
| 858 | H | CH₂—c-Pr | O | —CH₂—p-C₆H₄—CF₃ | CH₃ |
| 859 | c-Pr | | O | —CH₂—p-C₆H₄—CF₃ | CH₃ |
| 860 | c-Pentyl | H | O | —CH₂—p-C₆H₄—CF₃ | CH₃ |
| 861 | c-Bu | | O | Et | H |
| 862 | CH₂—c-Bu | H | O | Et | H |
| 863 | 5,5- | | O | Et | H |
| | spiro[2.3]hexane | | | | |
| 864 | H | nPr | O | Et | H |
| 865 | H | i-Pr | O | Et | H |
| 866 | H | nBu | O | Et | H |
| 867 | H | i-Bu | O | Et | H |
| 868 | H | CH₂—c-Pr | O | Et | H |
| 869 | c-Pr | | O | Et | H |
| 870 | c-Pentyl | H | O | Et | H |
| 871 | c-Bu | | O | Et | CF₃ |
| 872 | CH₂—c-Bu | H | O | Et | CF₃ |
| 873 | 5,5- | | O | Et | CF₃ |
| | spiro[2.3]hexane | | | | |
| 874 | H | nPr | O | Et | CF₃ |
| 875 | H | i-Pr | O | Et | CF₃ |
| 876 | H | nBu | O | Et | CF₃ |
| 877 | H | i-Bu | O | Et | CF₃ |
| 878 | H | CH₂—c-Pr | O | Et | CF₃ |
| 879 | c-Pr | | O | Et | CF₃ |
| 880 | c-Pentyl | H | O | Et | CF₃ |
| 881 | c-Bu | | O | Et | CH₃ |
| 882 | CH₂—c-Bu | H | O | Et | CH₃ |
| 883 | 5,5- | | O | Et | CH₃ |
| | spiro[2.3]hexane | | | | |
| 884 | H | nPr | O | Et | CH₃ |
| 885 | H | i-Pr | O | Et | CH₃ |
| 886 | H | nBu | O | Et | CH₃ |
| 887 | H | i-Bu | O | Et | CH₃ |
| 888 | H | CH₂—c-Pr | O | Et | CH₃ |
| 889 | c-Pr | | O | Et | CH₃ |
| 890 | c-Pentyl | H | O | Et | CH₃ |
| 891 | c-Bu | | O | CH₂CF₃ | H |
| 892 | CH₂—c-Bu | H | O | CH₂CF₃ | H |
| 893 | 5,5- | | O | CH₂CF₃ | H |
| | spiro[2.3]hexane | | | | |
| 894 | H | nPr | O | CH₂CF₃ | H |
| 895 | H | i-Pr | O | CH₂CF₃ | H |
| 896 | H | nBu | O | CH₂CF₃ | H |
| 897 | H | i-Bu | O | CH₂CF₃ | H |
| 898 | H | CH₂—c-Pr | O | CH₂CF₃ | H |
| 899 | c-Pr | | O | CH₂CF₃ | H |
| 900 | c-Pentyl | H | O | CH₂CF₃ | H |
| 901 | c-Bu | | O | CH₂CF₃ | CF₃ |
| 902 | CH₂—c-Bu | H | O | CH₂CF₃ | CF₃ |
| 903 | 5,5- | | O | CH₂CF₃ | CF₃ |
| | spiro[2.3]hexane | | | | |
| 904 | H | nPr | O | CH₂CF₃ | CF₃ |
| 905 | H | i-Pr | O | CH₂CF₃ | CF₃ |
| 906 | H | nBu | O | CH₂CF₃ | CF₃ |
| 907 | H | i-Bu | O | CH₂CF₃ | CF₃ |
| 908 | H | CH₂—c-Pr | O | CH₂CF₃ | CF₃ |
| 909 | c-Pr | | O | CH₂CF₃ | CF₃ |
| 910 | c-Pentyl | H | O | CH₂CF₃ | CF₃ |
| 911 | c-Bu | | O | CH₂CF₃ | CH₃ |
| 912 | CH₂—c-Bu | H | O | CH₂CF₃ | CH₃ |
| 913 | 5,5- | | O | CH₂CF₃ | CH₃ |
| | spiro[2.3]hexane | | | | |
| 914 | H | nPr | O | CH₂CF₃ | CH₃ |
| 915 | H | i-Pr | O | CH₂CF₃ | CH₃ |
| 916 | H | nBu | O | CH₂CF₃ | CH₃ |
| 917 | H | i-Bu | O | CH₂CF₃ | CH₃ |
| 918 | H | CH₂—c-Pr | O | CH₂CF₃ | CH₃ |
| 919 | c-Pr | | O | CH₂CF₃ | CH₃ |
| 920 | c-Pentyl | H | O | CH₂CF₃ | CH₃ |
| 921 | c-Bu | | O | CH₂CH₂OMe | H |
| 922 | CH₂—c-Bu | H | O | CH₂CH₂OMe | H |
| 923 | 5,5- | | O | CH₂CH₂OMe | H |
| | spiro[2.3]hexane | | | | |
| 924 | H | nPr | O | CH₂CH₂OMe | H |
| 925 | H | i-Pr | O | CH₂CH₂OMe | H |
| 926 | H | nBu | O | CH₂CH₂OMe | H |
| 927 | H | i-Bu | O | CH₂CH₂OMe | H |
| 928 | H | CH₂—c-Pr | O | CH₂CH₂OMe | H |
| 929 | c-Pr | | O | CH₂CH₂OMe | H |
| 930 | c-Pentyl | H | O | CH₂CH₂OMe | H |
| 931 | c-Bu | | O | CH₂CH₂OMe | CF₃ |
| 932 | CH₂—c-Bu | H | O | CH₂CH₂OMe | CF₃ |
| 933 | 5,5- | | O | CH₂CH₂OMe | CF₃ |
| | spiro[2.3]hexane | | | | |
| 934 | H | nPr | O | CH₂CH₂OMe | CF₃ |
| 935 | H | i-Pr | O | CH₂CH₂OMe | CF₃ |
| 936 | H | nBu | O | CH₂CH₂OMe | CF₃ |
| 937 | H | i-Bu | O | CH₂CH₂OMe | CF₃ |
| 938 | H | CH₂—c-Pr | O | CH₂CH₂OMe | CF₃ |
| 939 | c-Pr | | O | CH₂CH₂OMe | CF₃ |
| 940 | c-Pentyl | H | O | CH₂CH₂OMe | CF₃ |
| 941 | c-Bu | | O | CH₂CH₂OMe | CH₃ |
| 942 | CH₂—c-Bu | H | O | CH₂CH₂OMe | CH₃ |
| 943 | 5,5- | | O | CH₂CH₂OMe | CH₃ |
| | spiro[2.3]hexane | | | | |
| 944 | H | nPr | O | CH₂CH₂OMe | CH₃ |
| 945 | H | i-Pr | O | CH₂CH₂OMe | CH₃ |
| 946 | H | nBu | O | CH₂CH₂OMe | CH₃ |
| 947 | H | i-Bu | O | CH₂CH₂OMe | CH₃ |
| 948 | H | CH₂—c-Pr | O | CH₂CH₂OMe | CH₃ |
| 949 | c-Pr | | O | CH₂CH₂OMe | CH₃ |
| 950 | c-Pentyl | H | O | CH₂CH₂OMe | CH₃ |
| 951 | c-Bu | | — | Ph | H |
| 952 | CH₂—c-Bu | H | — | Ph | H |
| 953 | 5,5- | | — | Ph | H |
| | spiro[2.3]hexane | | | | |
| 954 | H | nPr | — | Ph | H |
| 955 | H | i-Pr | — | Ph | H |
| 956 | H | nBu | — | Ph | H |
| 957 | H | i-Bu | — | Ph | H |
| 958 | H | CH₂—c-Pr | — | Ph | H |
| 959 | c-Pr | | — | Ph | H |
| 960 | c-Pentyl | H | — | Ph | H |
| 961 | c-Bu | | — | Ph | CF₃ |
| 962 | CH₂—c-Bu | H | — | Ph | CF₃ |
| 963 | 5,5- | | — | Ph | CF₃ |
| | spiro[2.3]hexane | | | | |
| 964 | H | nPr | — | Ph | CF₃ |
| 965 | H | i-Pr | — | Ph | CF₃ |
| 966 | H | nBu | — | Ph | CF₃ |
| 967 | H | i-Bu | — | Ph | CF₃ |
| 968 | H | CH₂—c-Pr | — | Ph | CF₃ |
| 969 | c-Pr | | — | Ph | CF₃ |
| 970 | c-Pentyl | H | — | Ph | CF₃ |
| 971 | c-Bu | | — | Ph | CH₃ |
| 972 | CH₂—c-Bu | H | — | Ph | CH₃ |
| 973 | 5,5- | | — | Ph | CH₃ |
| | spiro[2.3]hexane | | | | |
| 974 | H | nPr | — | Ph | CH₃ |
| 975 | H | i-Pr | — | Ph | CH₃ |
| 976 | H | nBu | — | Ph | CH₃ |
| 977 | H | i-Bu | — | Ph | CH₃ |
| 978 | H | CH₂—c-Pr | — | Ph | CH₃ |
| 979 | c-Pr | | — | Ph | CH₃ |
| 980 | c-Pentyl | H | — | Ph | CH₃ |
| 981 | c-Bu | | — | p-C₆H₄—F | H |
| 982 | CH₂—c-Bu | H | — | p-C₆H₄—F | H |
| 983 | 5,5- | | — | p-C₆H₄—F | H |
| | spiro[2.3]hexane | | | | |
| 984 | H | nPr | — | p-C₆H₄—F | H |
| 985 | H | i-Pr | — | p-C₆H₄—F | H |
| 986 | H | nBu | — | p-C₆H₄—F | H |
| 987 | H | i-Bu | — | p-C₆H₄—F | H |
| 988 | H | CH₂—c-Pr | — | p-C₆H₄—F | H |
| 989 | c-Pr | | — | p-C₆H₄—F | H |
| 990 | c-Pentyl | H | — | p-C₆H₄—F | H |
| 991 | c-Bu | | — | p-C₆H₄—F | CF₃ |
| 992 | CH₂—c-Bu | H | — | p-C₆H₄—F | CF₃ |
| 993 | 5,5- | | — | CF₃ | |
| | spiro[2.3]hexane | | | | |
| 994 | H | nPr | — | p-C₆H₄—F | CF₃ |
| 995 | H | i-Pr | — | p-C₆H₄—F | CF₃ |
| 996 | H | nBu | — | p-C₆H₄—F | CF₃ |
| 997 | H | i-Bu | — | p-C₆H₄—F | CF₃ |
| 998 | H | CH₂—c-Pr | — | p-C₆H₄—F | CF₃ |
| 999 | c-Pr | | — | p-C₆H₄—F | CF₃ |
| 1000 | c-Pentyl | H | — | p-C₆H₄—F | CF₃ |
| 1001 | c-Bu | | — | p-C₆H₄—F | CH₃ |
| 1002 | CH₂—c-Bu | H | — | p-C₆H₄—F | CH₃ |
| 1003 | 5,5- | | — | p-C₆H₄—F | CH₃ |
| | spiro[2.3]hexane | | | | |
| 1004 | H | nPr | — | p-C₆H₄—F | CH₃ |
| 1005 | H | i-Pr | — | p-C₆H₄—F | CH₃ |
| 1006 | H | nBu | — | p-C₆H₄—F | CH₃ |
| 1007 | H | i-Bu | — | p-C₆H₄—F | CH₃ |
| 1008 | H | CH₂—c-Pr | — | p-C₆H₄—F | CH₃ |
| 1009 | c-Pr | | — | p-C₆H₄—F | CH₃ |
| 1010 | c-Pentyl | H | — | p-C₆H₄—F | CH₃ |
| 1011 | c-Bu | | — | p-C₆H₄—Cl | H |
| 1012 | CH₂—c-Bu | H | — | p-C₆H₄—Cl | H |
| 1013 | 5,5- | | — | H | |
| | spiro[2.3]hexane | | | | |
| 1014 | H | nPr | — | p-C₆H₄—Cl | H |
| 1015 | H | i-Pr | — | p-C₆H₄—Cl | H |
| 1016 | H | nBu | — | p-C₆H₄—Cl | H |
| 1017 | H | i-Bu | — | p-C₆H₄—Cl | H |
| 1018 | H | CH₂—c-Pr | — | p-C₆H₄—Cl | H |
| 1019 | c-Pr | | — | p-C₆H₄—Cl | H |
| 1020 | c-Pentyl | H | — | p-C₆H₄—Cl | H |
| 1021 | c-Bu | | — | p-C₆H₄—Cl | CF₃ |
| 1022 | CH₂—c-Bu | H | — | p-C₆H₄—Cl | CF₃ |
| 1023 | 5,5- | | — | p-C₆H₄—Cl | CF₃ |
| | spiro[2.3]hexane | | | | |
| 1024 | H | nPr | — | p-C₆H₄—Cl | CF₃ |
| 1025 | H | i-Pr | — | p-C₆H₄—Cl | CF₃ |
| 1026 | H | nBu | — | p-C₆H₄—Cl | CF₃ |
| 1027 | H | i-Bu | — | p-C₆H₄—Cl | CF₃ |
| 1028 | H | CH₂—c-Pr | — | p-C₆H₄—Cl | CF₃ |
| 1029 | c-Pr | | — | p-C₆H₄—Cl | CF₃ |
| 1030 | c-Pentyl | H | — | p-C₆H₄—Cl | CF₃ |
| 1031 | c-Bu | | — | p-C₆H₄—Cl | CH₃ |
| 1032 | CH₂—c-Bu | H | — | p-C₆H₄—Cl | CH₃ |
| 1033 | 5,5- | | — | p-C₆H₄—Cl | CH₃ |
| | spiro[2.3]hexane | | | | |
| 1034 | H | nPr | — | p-C₆H₄—Cl | CH₃ |
| 1035 | H | i-Pr | — | p-C₆H₄—Cl | CH₃ |
| 1036 | H | nBu | — | p-C₆H₄—Cl | CH₃ |
| 1037 | H | i-Bu | — | p-C₆H₄—Cl | CH₃ |
| 1038 | H | CH₂—c-Pr | — | p-C₆H₄—Cl | CH₃ |
| 1039 | c-Pr | | — | p-C₆H₄—Cl | CH₃ |
| 1040 | c-Pentyl | H | — | p-C₆H₄—Cl | CH₃ |
| 1041 | c-Bu | | — | p-C₆H₄—CF₃ | H |
| 1042 | CH₂—c-Bu | H | — | p-C₆H₄—CF₃ | H |
| 1043 | 5,5- | | — | p-C₆H₄—CF₃ | H |
| | spiro[2.3]hexane | | | | |
| 1044 | H | nPr | — | p-C₆H₄—CF₃ | H |
| 1045 | H | i-Pr | — | p-C₆H₄—CF₃ | H |
| 1046 | H | nBu | — | p-C₆H₄—CF₃ | H |
| 1047 | H | i-Bu | — | p-C₆H₄—CF₃ | H |
| 1048 | H | CH₂—c-Pr | — | p-C₆H₄—CF₃ | H |
| 1049 | c-Pr | | — | p-C₆H₄—CF₃ | H |
| 1050 | c-Pentyl | H | — | p-C₆H₄—CF₃ | H |
| 1051 | c-Bu | | — | p-C₆H₄—CF₃ | CF₃ |
| 1052 | CH₂—c-Bu | H | — | p-C₆H₄—CF₃ | CF₃ |
| 1053 | 5,5- | | — | p-C₆H₄—CF₃ | CF₃ |
| | spiro[2.3]hexane | | | | |
| 1054 | H | nPr | — | p-C₆H₄—CF₃ | CF₃ |
| 1055 | H | i-Pr | — | p-C₆H₄—CF₃ | CF₃ |
| 1056 | H | nBu | — | p-C₆H₄—CF₃ | CF₃ |
| 1057 | H | i-Bu | — | p-C₆H₄—CF₃ | CF₃ |
| 1058 | H | CH₂—c-Pr | — | p-C₆H₄—CF₃ | CF₃ |
| 1059 | c-Pr | | — | p-C₆H₄—CF₃ | CF₃ |
| 1060 | c-Pentyl | H | — | p-C₆H₄—CF₃ | CF₃ |
| 1061 | c-Bu | | — | p-C₆H₄—CF₃ | CH₃ |
| 1062 | CH₂—c-Bu | H | — | p-C₆H₄—CF₃ | CH₃ |
| 1063 | 5,5- | | — | p-C₆H₄—CF₃ | CH₃ |
| | spiro[2.3]hexane | | | | |
| 1064 | H | nPr | — | p-C₆H₄—CF₃ | CH₃ |
| 1065 | H | i-Pr | — | p-C₆H₄—CF₃ | CH₃ |
| 1066 | H | nBu | — | p-C₆H₄—CF₃ | CH₃ |
| 1067 | H | i-Bu | — | p-C₆H₄—CF₃ | CH₃ |
| 1068 | H | CH₂—c-Pr | | p-C₆H₄—CF₃ | CH₃ |
| 1069 | c-Pr | | — | p-C₆H₄—CF₃ | CH₃ |
| 1070 | c-Pentyl | H | — | p-C₆H₄—CF₃ | CH₃ |

A compound of formula (VIII) where

TABLE 5
(VIII)

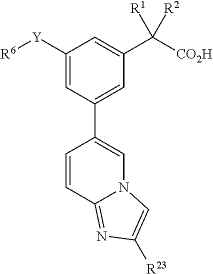

| Cpd # | R¹ | | R² | Y | R⁶ | R²³ |
|---|---|---|---|---|---|---|
| 1071 | c-Bu | | | O | —CH₂-cyclopropyl | H |
| 1072 | CH₂—c-Bu | | H | O | —CH₂-cyclopropyl | H |
| 1073 | 5,5-spiro | | | O | —CH₂-cyclopropyl | H |
| | [2.3]hexane | | | | | |
| 1074 | H | | nPr | O | —CH₂-cyclopropyl | H |
| 1075 | H | | i-Pr | O | —CH₂-cyclopropyl | H |
| 1076 | H | | nBu | O | —CH₂-cyclopropyl | H |
| 1077 | H | | i-Bu | O | —CH₂-cyclopropyl | H |
| 1078 | H | | CH₂—c-Pr | O | —CH₂-cyclopropyl | H |
| 1079 | | c-Pr | | O | —CH₂-cyclopropyl | H |
| 1080 | c-Pentyl | | H | O | —CH₂-cyclopropyl | H |
| 1081 | c-Bu | | | O | —CH₂-cyclopropyl | CF₃ |
| 1082 | CH₂—c-Bu | | H | O | —CH₂-cyclopropyl | CF₃ |
| 1083 | 5,5-spiro | | | O | —CH₂-cyclopropyl | CF₃ |
| | [2.3]hexane | | | | | |
| 1084 | H | | nPr | O | —CH₂-cyclopropyl | CF₃ |
| 1085 | H | | i-Pr | O | —CH₂-cyclopropyl | CF₃ |
| 1086 | H | | nBu | O | —CH₂-cyclopropyl | CF₃ |
| 1087 | H | | i-Bu | O | —CH₂-cyclopropyl | CF₃ |
| 1088 | H | | CH₂—c-Pr | O | —CH₂-cyclopropyl | CF₃ |
| 1089 | | c-Pr | | O | —CH₂-cyclopropyl | CF₃ |
| 1090 | c-Pentyl | | H | O | —CH₂-cyclopropyl | CF₃ |
| 1091 | c-Bu | | | O | —CH₂-cyclopropyl | CH₃ |
| 1092 | CH₂—c-Bu | | H | O | —CH₂-cyclopropyl | CH₃ |
| 1093 | 5,5-spiro | | | O | —CH₂-cyclopropyl | CH₃ |
| | [2.3]hexane | | | | | |
| 1094 | H | | nPr | O | —CH₂-cyclopropyl | CH₃ |
| 1095 | H | | i-Pr | O | —CH₂-cyclopropyl | CH₃ |
| 1096 | H | | nBu | O | —CH₂-cyclopropyl | CH₃ |
| 1097 | H | | i-Bu | O | —CH₂-cyclopropyl | CH₃ |
| 1098 | H | | CH₂—c-Pr | O | —CH₂-cyclopropyl | CH₃ |
| 1099 | | c-Pr | | O | —CH₂-cyclopropyl | CH₃ |
| 1100 | c-Pentyl | | H | O | —CH₂-cyclopropyl | CH₃ |
| 1101 | c-Bu | | | O | —CH₂—p-C₆H₄—F | H |
| 1102 | CH₂—c-Bu | | H | O | —CH₂—p-C₆H₄—F | H |
| 1103 | 5,5-spiro | | | O | —CH₂—p-C₆H₄—F | H |
| | [2.3]hexane | | | | | |
| 1104 | H | | nPr | O | —CH₂—p-C₆H₄—F | H |
| 1105 | H | | i-Pr | O | —CH₂—p-C₆H₄—F | H |
| 1106 | H | | nBu | O | —CH₂—p-C₆H₄—F | H |
| 1107 | H | | i-Bu | O | —CH₂—p-C₆H₄—F | H |
| 1108 | H | | CH₂—c-Pr | O | —CH₂—p-C₆H₄—F | H |
| 1109 | | c-Pr | | O | —CH₂—p-C₆H₄—F | H |
| 1110 | c-Pentyl | | H | O | —CH₂—p-C₆H₄—F | H |
| 1111 | c-Bu | | | O | —CH₂—p-C₆H₄—F | CF₃ |
| 1112 | CH₂—c-Bu | | H | O | —CH₂—p-C₆H₄—F | CF₃ |
| 1113 | 5,5-spiro | | | O | —CH₂—p-C₆H₄—F | CF₃ |
| | [2.3]hexane | | | | | |
| 1114 | H | | nPr | O | —CH₂—p-C₆H₄—F | CF₃ |
| 1115 | H | | i-Pr | O | —CH₂—p-C₆H₄—F | CF₃ |
| 1116 | H | | nBu | O | —CH₂—p-C₆H₄—F | CF₃ |
| 1117 | H | | i-Bu | O | —CH₂—p-C₆H₄—F | CF₃ |
| 1118 | H | | CH₂—c-Pr | O | —CH₂—p-C₆H₄—F | CF₃ |
| 1119 | | c-Pr | | O | —CH₂—p-C₆H₄—F | CF₃ |
| 1120 | c-Pentyl | | H | O | —CH₂—p-C₆H₄—F | CF₃ |
| 1121 | c-Bu | | | O | —CH₂—p-C₆H₄—F | CH₃ |
| 1122 | CH₂—c-Bu | | H | O | —CH₂—p-C₆H₄—F | CH₃ |
| 1123 | 5,5-spiro | | | O | —CH₂—p-C₆H₄—F | CH₃ |
| | [2.3]hexane | | | | | |
| 1124 | H | | nPr | O | —CH₂—p-C₆H₄—F | CH₃ |
| 1125 | H | | i-Pr | O | —CH₂—p-C₆H₄—F | CH₃ |
| 1126 | H | | nBu | O | —CH₂—p-C₆H₄—F | CH₃ |
| 1127 | H | | i-Bu | O | —CH₂—p-C₆H₄—F | CH₃ |
| 1128 | H | | CH₂—c-Pr | O | —CH₂—p-C₆H₄—F | CH₃ |
| 1129 | | c-Pr | | O | —CH₂—p-C₆H₄—F | CH₃ |
| 1130 | c-Pentyl | | H | O | —CH₂—p-C₆H₄—F | CH₃ |
| 1131 | c-Bu | | | O | —CH₂—p-C₆H₄—Cl | H |
| 1132 | CH₂—c-Bu | | H | O | —CH₂—p-C₆H₄—Cl | H |
| 1133 | 5,5-spiro | | | O | —CH₂—p-C₆H₄—Cl | H |
| | [2.3]hexane | | | | | |
| 1134 | H | | nPr | O | —CH₂—p-C₆H₄—Cl | H |
| 1135 | H | | i-Pr | O | —CH₂—p-C₆H₄—Cl | H |
| 1136 | H | | nBu | O | —CH₂—p-C₆H₄—Cl | H |
| 1137 | H | | i-Bu | O | —CH₂—p-C₆H₄—Cl | H |
| 1138 | H | | CH₂—c-Pr | O | —CH₂—p-C₆H₄—Cl | H |
| 1139 | | c-Pr | | O | —CH₂—p-C₆H₄—Cl | H |
| 1140 | c-Pentyl | | H | O | —CH₂—p-C₆H₄—Cl | H |
| 1141 | c-Bu | | | O | —CH₂—p-C₆H₄—Cl | CF₃ |
| 1142 | CH₂—c-Bu | | H | O | —CH₂—p-C₆H₄—Cl | CF₃ |
| 1143 | 5,5-spiro | | | O | —CH₂—p-C₆H₄—Cl | CF₃ |
| | [2.3]hexane | | | | | |
| 1144 | H | | nPr | O | —CH₂—p-C₆H₄—Cl | CF₃ |
| 1145 | H | | i-Pr | O | —CH₂—p-C₆H₄—Cl | CF₃ |
| 1146 | H | | nBu | O | —CH₂—p-C₆H₄—Cl | CF₃ |
| 1147 | H | | i-Bu | O | —CH₂—p-C₆H₄—Cl | CF₃ |
| 1148 | H | | CH₂—c-Pr | O | —CH₂—p-C₆H₄—Cl | CF₃ |
| 1149 | | c-Pr | | O | —CH₂—p-C₆H₄—Cl | CF₃ |
| 1150 | c-Pentyl | | H | O | —CH₂—p-C₆H₄—Cl | CF₃ |
| 1151 | c-Bu | | | O | —CH₂—p-C₆H₄—Cl | CH₃ |
| 1152 | CH₂—c-Bu | | H | O | —CH₂—p-C₆H₄—Cl | CH₃ |
| 1153 | 5,5-spiro | | | O | —CH₂—p-C₆H₄—Cl | CH₃ |
| | [2.3]hexane | | | | | |
| 1154 | H | | nPr | O | —CH₂—p-C₆H₄—Cl | CH₃ |
| 1155 | H | | i-Pr | O | —CH₂—p-C₆H₄—Cl | CH₃ |
| 1156 | H | | nBu | O | —CH₂—p-C₆H₄—Cl | CH₃ |
| 1157 | H | | i-Bu | O | —CH₂—p-C₆H₄—Cl | CH₃ |
| 1158 | H | | CH₂—c-Pr | O | —CH₂—p-C₆H₄—Cl | CH₃ |
| 1159 | | c-Pr | | O | —CH₂—p-C₆H₄—Cl | CH₃ |
| 1160 | c-Pentyl | | H | O | —CH₂—p-C₆H₄—Cl | CH₃ |
| 1161 | c-Bu | | | O | —CH₂—p-C₆H₄—CF₃ | H |
| 1162 | CH₂—c-Bu | | H | O | —CH₂—p-C₆H₄—CF₃ | H |
| 1163 | 5,5-spiro | | | O | —CH₂—p-C₆H₄—CF₃ | H |
| | [2.3]hexane | | | | | |
| 1164 | H | | nPr | O | —CH₂—p-C₆H₄—CF₃ | H |
| 1165 | H | | i-Pr | O | —CH₂—p-C₆H₄—CF₃ | H |
| 1166 | H | | nBu | O | —CH₂—p-C₆H₄—CF₃ | H |
| 1167 | H | | i-Bu | O | —CH₂—p-C₆H₄—CF₃ | H |
| 1168 | H | | CH₂—c-Pr | O | —CH₂—p-C₆H₄—CF₃ | H |
| 1169 | | c-Pr | | O | —CH₂—p-C₆H₄—CF₃ | H |
| 1170 | c-Pentyl | | H | O | —CH₂—p-C₆H₄—CF₃ | H |
| 1171 | c-Bu | | | O | —CH₂—p-C₆H₄—CF₃ | CF₃ |
| 1172 | CH₂—c-Bu | | H | O | —CH₂—p-C₆H₄—CF₃ | CF₃ |
| 1173 | 5,5-spiro | | | O | —CH₂—p-C₆H₄—CF₃ | CF₃ |
| | [2.3]hexane | | | | | |
| 1174 | H | | nPr | O | —CH₂—p-C₆H₄—CF₃ | CF₃ |
| 1175 | H | | i-Pr | O | —CH₂—p-C₆H₄—CF₃ | CF₃ |
| 1176 | H | | nBu | O | —CH₂—p-C₆H₄—CF₃ | CF₃ |
| 1177 | H | | i-Bu | O | —CH₂—p-C₆H₄—CF₃ | CF₃ |
| 1178 | H | | CH₂—c-Pr | O | —CH₂—p-C₆H₄—CF₃ | CF₃ |
| 1179 | | c-Pr | | O | —CH₂—p-C₆H₄—CF₃ | CF₃ |
| 1180 | c-Pentyl | | H | O | —CH₂—p-C₆H₄—CF₃ | CF₃ |
| 1181 | c-Bu | | | O | —CH₂—p-C₆H₄—CF₃ | CH₃ |
| 1182 | CH₂—c-Bu | | H | O | —CH₂—p-C₆H₄—CF₃ | CH₃ |
| 1183 | 5,5-spiro | | | O | —CH₂—p-C₆H₄—CF₃ | CH₃ |
| | [2.3]hexane | | | | | |
| 1184 | H | | nPr | O | —CH₂—p-C₆H₄—CF₃ | CH₃ |
| 1185 | H | | i-Pr | O | —CH₂—p-C₆H₄—CF₃ | CH₃ |
| 1186 | H | | nBu | O | —CH₂—p-C₆H₄—CF₃ | CH₃ |
| 1187 | H | | i-Bu | O | —CH₂—p-C₆H₄—CF₃ | CH₃ |
| 1188 | H | | CH₂—c-Pr | O | —CH₂—p-C₆H₄—CF₃ | CH₃ |
| 1189 | | c-Pr | | O | —CH₂—p-C₆H₄—CF₃ | CH₃ |
| 1190 | c-Pentyl | | H | O | —CH₂—p-C₆H₄—CF₃ | CH₃ |
| 1191 | c-Bu | | | O | Et | H |
| 1192 | CH₂—c-Bu | | H | O | Et | H |
| 1193 | 5,5-spiro | | | O | Et | H |
| | [2.3]hexane | | | | | |
| 1194 | H | | nPr | O | Et | H |
| 1195 | H | | i-Pr | O | Et | H |
| 1196 | H | | nBu | O | Et | H |
| 1197 | H | | i-Bu | O | Et | H |
| 1198 | H | | CH₂—c-Pr | O | Et | H |
| 1199 | | c-Pr | | O | Et | H |
| 1200 | c-Pentyl | | H | O | Et | H |
| 1201 | c-Bu | | | O | Et | CF₃ |
| 1202 | CH₂—c-Bu | | H | O | Et | CF₃ |
| 1203 | 5,5-spiro | | | O | Et | CF₃ |
| | [2.3]hexane | | | | | |
| 1204 | H | | nPr | O | Et | CF₃ |
| 1205 | H | | i-Pr | O | Et | CF₃ |
| 1206 | H | | nBu | O | Et | CF₃ |
| 1207 | H | | i-Bu | O | Et | CF₃ |
| 1208 | H | | CH₂—c-Pr | O | Et | CF₃ |
| 1209 | | c-Pr | | O | Et | CF₃ |
| 1210 | c-Pentyl | | H | O | Et | CF₃ |
| 1211 | c-Bu | | | O | Et | CH₃ |
| 1212 | CH₂—c-Bu | | H | O | Et | CH₃ |
| 1213 | 5,5-spiro | | | O | Et | CH₃ |
| | [2.3]hexane | | | | | |
| 1214 | H | | nPr | O | Et | CH₃ |
| 1215 | H | | i-Pr | O | Et | CH₃ |
| 1216 | H | | nBu | O | Et | CH₃ |
| 1217 | H | | i-Bu | O | Et | CH₃ |
| 1218 | H | | CH₂—c-Pr | O | Et | CH₃ |
| 1219 | | c-Pr | | O | Et | CH₃ |
| 1220 | c-Pentyl | | H | O | Et | CH₃ |
| 1221 | c-Bu | | | O | CH₂CF₃ | H |
| 1222 | CH₂—c-Bu | | H | O | CH₂CF₃ | H |
| 1223 | 5,5-spiro | | | O | CH₂CF₃ | H |
| | [2.3]hexane | | | | | |
| 1224 | H | | nPr | O | CH₂CF₃ | H |
| 1225 | H | | i-Pr | O | CH₂CF₃ | H |
| 1226 | H | | nBu | O | CH₂CF₃ | H |
| 1227 | H | | i-Bu | O | CH₂CF₃ | H |
| 1228 | H | | CH₂—c-Pr | O | CH₂CF₃ | H |
| 1229 | | c-Pr | | O | CH₂CF₃ | H |
| 1230 | c-Pentyl | | H | O | CH₂CF₃ | H |
| 1231 | c-Bu | | | O | CH₂CF₃ | CF₃ |
| 1232 | CH₂—c-Bu | | H | O | CH₂CF₃ | CF₃ |
| 1233 | 5,5-spiro | | | O | CH₂CF₃ | CF₃ |
| | [2.3]hexane | | | | | |
| 1234 | H | | nPr | O | CH₂CF₃ | CF₃ |
| 1235 | H | | i-Pr | O | CH₂CF₃ | CF₃ |
| 1236 | H | | nBu | O | CH₂CF₃ | CF₃ |
| 1237 | H | | i-Bu | O | CH₂CF₃ | CF₃ |
| 1238 | H | | CH₂—c-Pr | O | CH₂CF₃ | CF₃ |
| 1239 | | c-Pr | | O | CH₂CF₃ | CF₃ |
| 1240 | c-Pentyl | | H | O | CH₂CF₃ | CF₃ |
| 1241 | c-Bu | | | O | CH₂CF₃ | CH₃ |
| 1242 | CH₂—c-Bu | | H | O | CH₂CF₃ | CH₃ |
| 1243 | 5,5-spiro | | | O | CH₂CF₃ | CH₃ |
| | [2.3]hexane | | | | | |
| 1244 | H | | nPr | O | CH₂CF₃ | CH₃ |
| 1245 | H | | i-Pr | O | CH₂CF₃ | CH₃ |
| 1246 | H | | nBu | O | CH₂CF₃ | CH₃ |
| 1247 | H | | i-Bu | O | CH₂CF₃ | CH₃ |
| 1248 | H | | CH₂—c-Pr | O | CH₂CF₃ | CH₃ |
| 1249 | | c-Pr | | O | CH₂CF₃ | CH₃ |
| 1250 | c-Pentyl | | H | O | CH₂CF₃ | CH₃ |
| 1251 | c-Bu | | | O | CH₂CH₂OMe | H |
| 1252 | CH₂-c-Bu | | H | O | CH₂CH₂OMe | H |
| 1253 | 5,5-spiro | | | O | CH₂CH₂OMe | H |
| | [2.3]hexane | | | | | |
| 1254 | H | | nPr | O | CH₂CH₂OMe | H |
| 1255 | H | | i-Pr | O | CH₂CH₂OMe | H |
| 1256 | H | | nBu | O | CH₂CH₂OMe | H |
| 1257 | H | | i-Bu | O | CH₂CH₂OMe | H |
| 1258 | H | | CH₂—c-Pr | O | CH₂CH₂OMe | H |
| 1259 | | c-Pr | | O | CH₂CH₂OMe | H |
| 1260 | c-Pentyl | | H | O | CH₂CH₂OMe | H |
| 1261 | c-Bu | | | O | CH₂CH₂OMe | CF₃ |
| 1262 | CH₂—c-Bu | | H | O | CH₂CH₂OMe | CF₃ |
| 1263 | 5,5-spiro | | | O | CH₂CH₂OMe | CF₃ |
| | [2.3]hexane | | | | | |
| 1264 | H | | nPr | O | CH₂CH₂OMe | CF₃ |
| 1265 | H | | i-Pr | O | CH₂CH₂OMe | CF₃ |
| 1266 | H | | nBu | O | CH₂CH₂OMe | CF₃ |
| 1267 | H | | i-Bu | O | CH₂CH₂OMe | CF₃ |
| 1268 | H | | CH₂—c-Pr | O | CH₂CH₂OMe | CF₃ |
| 1269 | | c-Pr | | O | CH₂CH₂OMe | CF₃ |
| 1270 | c-Pentyl | | H | O | CH₂CH₂OMe | CF₃ |
| 1271 | c-Bu | | | O | CH₂CH₂OMe | CH₃ |
| 1272 | CH₂—c-Bu | | H | O | CH₂CH₂OMe | CH₃ |
| 1273 | 5,5-spiro | | | O | CH₂CH₂OMe | CH₃ |
| | [2.3]hexane | | | | | |
| 1274 | H | | nPr | O | CH₂CH₂OMe | CH₃ |
| 1275 | H | | i-Pr | O | CH₂CH₂OMe | CH₃ |
| 1276 | H | | nBu | O | CH₂CH₂OMe | CH₃ |
| 1277 | H | | i-Bu | O | CH₂CH₂OMe | CH₃ |
| 1278 | H | | CH₂—c-Pr | O | CH₂CH₂OMe | CH₃ |
| 1279 | | c-Pr | | O | CH₂CH₂OMe | CH₃ |
| 1280 | c-Pentyl | | H | O | CH₂CH₂OMe | CH₃ |
| 1281 | c-Bu | | | O | Ph | H |
| 1282 | CH₂—c-Bu | | H | O | Ph | H |
| 1283 | 5,5-spiro | | | O | Ph | H |
| | [2.3]hexane | | | | | |
| 1284 | H | | nPr | O | Ph | H |
| 1285 | H | | i-Pr | O | Ph | H |
| 1286 | H | | nBu | O | Ph | H |
| 1287 | H | | i-Bu | O | Ph | H |
| 1288 | H | | CH₂—c-Pr | O | Ph | H |
| 1289 | | c-Pr | | O | Ph | H |
| 1290 | c-Pentyl | | H | O | Ph | H |
| 1291 | c-Bu | | | O | Ph | CF₃ |
| 1292 | CH₂—c-Bu | | H | O | Ph | CF₃ |
| 1293 | 5,5-spiro | | | O | Ph | CF₃ |
| | [2.3]hexane | | | | | |
| 1294 | H | | nPr | O | Ph | CF₃ |
| 1295 | H | | i-Pr | O | Ph | CF₃ |
| 1296 | H | | nBu | O | Ph | CF₃ |
| 1297 | H | | i-Bu | O | Ph | CF₃ |
| 1298 | H | | CH₂—c-Pr | O | Ph | CF₃ |
| 1299 | | c-Pr | | O | Ph | CF₃ |
| 1300 | c-Pentyl | | H | O | Ph | CF₃ |
| 1301 | c-Bu | | | O | Ph | CH₃ |
| 1302 | CH₂—c-Bu | | H | O | Ph | CH₃ |
| 1303 | 5,5-spiro | | | O | Ph | CH₃ |
| | [2.3]hexane | | | | | |
| 1304 | H | | nPr | O | Ph | CH₃ |
| 1305 | H | | i-Pr | O | Ph | CH₃ |
| 1306 | H | | nBu | O | Ph | CH₃ |
| 1307 | H | | i-Bu | O | Ph | CH₃ |
| 1308 | H | | CH₂—c-Pr | O | Ph | CH₃ |
| 1309 | | c-Pr | | O | Ph | CH₃ |
| 1310 | c-Pentyl | | H | O | Ph | CH₃ |
| 1311 | c-Bu | | | O | p-C₆H₄—F | H |
| 1312 | CH₂—c-Bu | | H | O | p-C₆H₄—F | H |
| 1313 | 5,5-spiro | | | O | p-C₆H₄—F | H |
| | [2.3]hexane | | | | | |
| 1314 | H | | nPr | O | p-C₆H₄—F | H |
| 1315 | H | | i-Pr | O | p-C₆H₄—F | H |
| 1316 | H | | nBu | O | p-C₆H₄—F | H |
| 1317 | H | | i-Bu | O | p-C₆H₄—F | H |
| 1318 | H | | CH₂—c-Pr | O | p-C₆H₄—F | H |
| 1319 | | c-Pr | | O | p-C₆H₄—F | H |
| 1320 | c-Pentyl | | H | O | p-C₆H₄—F | H |
| 1321 | c-Bu | | | O | p-C₆H₄—F | CF₃ |
| 1322 | CH₂—c-Bu | | H | O | p-C₆H₄—F | CF₃ |
| 1323 | 5,5-spiro | | | O | p-C₆H₄—F | CF₃ |
| | [2.3]hexane | | | | | |
| 1324 | H | | nPr | O | p-C₆H₄—F | CF₃ |
| 1325 | H | | i-Pr | O | p-C₆H₄—F | CF₃ |
| 1326 | H | | nBu | O | p-C₆H₄—F | CF₃ |
| 1327 | H | | i-Bu | O | p-C₆H₄—F | CF₃ |
| 1328 | H | | CH₂—c-Pr | O | p-C₆H₄—F | CF₃ |
| 1329 | | c-Pr | | O | p-C₆H₄—F | CF₃ |
| 1330 | c-Pentyl | | H | O | p-C₆H₄—F | CF₃ |
| 1331 | c-Bu | | | O | p-C₆H₄—F | CH₃ |
| 1332 | CH₂—c-Bu | | H | O | p-C₆H₄—F | CH₃ |
| 1333 | 5,5-spiro | | | O | p-C₆H₄—F | CH₃ |
| | [2.3]hexane | | | | | |
| 1334 | H | | nPr | O | p-C₆H₄—F | CH₃ |
| 1335 | H | | i-Pr | O | p-C₆H₄—F | CH₃ |
| 1336 | H | | nBu | O | p-C₆H₄—F | CH₃ |
| 1337 | H | | i-Bu | O | p-C₆H₄—F | CH₃ |
| 1338 | H | | CH₂—c-Pr | O | p-C₆H₄—F | CH₃ |
| 1339 | | c-Pr | | O | p-C₆H₄—F | CH₃ |
| 1340 | c-Pentyl | | H | O | p-C₆H₄—F | CH₃ |
| 1341 | c-Bu | | | O | p-C₆H₄—Cl | H |
| 1342 | CH₂—c-Bu | | H | O | p-C₆H₄—Cl | H |
| 1343 | 5,5-spiro | | | O | p-C₆H₄—Cl | H |
| | [2.3]hexane | | | | | |
| 1344 | H | | nPr | O | p-C₆H₄—Cl | H |
| 1345 | H | | i-Pr | O | p-C₆H₄—Cl | H |
| 1346 | H | | nBu | O | p-C₆H₄—Cl | H |
| 1347 | H | | i-Bu | O | p-C₆H₄—Cl | H |
| 1348 | H | | CH₂—c-Pr | O | p-C₆H₄—Cl | H |
| 1349 | | c-Pr | | O | p-C₆H₄—Cl | H |
| 1350 | c-Pentyl | | H | O | p-C₆H₄—Cl | H |
| 1351 | c-Bu | | | O | p-C₆H₄—Cl | CF₃ |
| 1352 | CH₂—c-Bu | | H | O | p-C₆H₄—Cl | CF₃ |
| 1353 | 5,5-spiro | | | O | p-C₆H₄—Cl | CF₃ |
| | [2.3]hexane | | | | | |
| 1354 | H | | nPr | O | p-C₆H₄—Cl | CF₃ |
| 1355 | H | | i-Pr | O | p-C₆H₄—Cl | CF₃ |
| 1356 | H | | nBu | O | p-C₆H₄—Cl | CF₃ |
| 1357 | H | | i-Bu | O | p-C₆H₄—Cl | CF₃ |
| 1358 | H | | CH₂—c-Pr | O | p-C₆H₄—Cl | CF₃ |
| 1359 | | c-Pr | | O | p-C₆H₄—Cl | CF₃ |
| 1360 | c-Pentyl | | H | O | p-C₆H₄—Cl | CF₃ |
| 1361 | c-Bu | | | O | p-C₆H₄—Cl | CH₃ |
| 1362 | CH₂—c-Bu | | H | O | p-C₆H₄—Cl | CH₃ |
| 1363 | 5,5-spiro | | | O | p-C₆H₄—Cl | CH₃ |
| | [2.3]hexane | | | | | |
| 1364 | H | | nPr | O | p-C₆H₄—Cl | CH₃ |
| 1365 | H | | i-Pr | O | p-C₆H₄—Cl | CH₃ |
| 1366 | H | | nBu | O | p-C₆H₄—Cl | CH₃ |
| 1367 | H | | i-Bu | O | p-C₆H₄—Cl | CH₃ |
| 1368 | H | | CH₂—c-Pr | O | p-C₆H₄—Cl | CH₃ |
| 1369 | | c-Pr | | O | p-C₆H₄—Cl | CH₃ |
| 1370 | c-Pentyl | | H | O | p-C₆H₄—Cl | CH₃ |
| 1371 | c-Bu | | | O | p-C₆H₄—CF₃ | H |
| 1372 | CH₂—c-Bu | | H | O | p-C₆H₄—CF₃ | H |
| 1373 | 5,5-spiro | | | O | p-C₆H₄—CF₃ | H |
| | [2.3]hexane | | | | | |
| 1374 | H | | nPr | O | p-C₆H₄—CF₃ | H |
| 1375 | H | | i-Pr | O | p-C₆H₄—CF₃ | H |
| 1376 | H | | nBu | O | p-C₆H₄—CF₃ | H |
| 1377 | H | | i-Bu | O | p-C₆H₄—CF₃ | H |
| 1378 | H | | CH₂—c-Pr | O | p-C₆H₄—CF₃ | H |
| 1379 | | c-Pr | | O | p-C₆H₄—CF₃ | H |
| 1380 | c-Pentyl | | H | O | p-C₆H₄—CF₃ | H |
| 1381 | c-Bu | | | O | p-C₆H₄—CF₃ | CF₃ |
| 1382 | CH₂—c-Bu | | H | O | p-C₆H₄—CF₃ | CF₃ |
| 1383 | 5,5-spiro | | | O | p-C₆H₄—CF₃ | CF₃ |
| | [2.3]hexane | | | | | |
| 1384 | H | | nPr | O | p-C₆H₄—CF₃ | CF₃ |
| 1385 | H | | i-Pr | O | p-C₆H₄—CF₃ | CF₃ |
| 1386 | H | | nBu | O | p-C₆H₄—CF₃ | CF₃ |
| 1387 | H | | i-Bu | O | p-C₆H₄—CF₃ | CF₃ |
| 1388 | H | | CH₂—c-Pr | O | p-C₆H₄—CF₃ | CF₃ |
| 1389 | | c-Pr | | O | p-C₆H₄—CF₃ | CF₃ |
| 1390 | c-Pentyl | | H | O | p-C₆H₄—CF₃ | CF₃ |
| 1391 | c-Bu | | | O | p-C₆H₄—CF₃ | CH₃ |
| 1392 | CH₂—c-Bu | | H | O | p-C₆H₄—CF₃ | CH₃ |
| 1393 | 5,5-spiro | | | O | p-C₆H₄—CF₃ | CH₃ |
| | [2.3]hexane | | | | | |
| 1394 | H | | nPr | O | p-C₆H₄—CF₃ | CH₃ |
| 1395 | H | | i-Pr | O | p-C₆H₄—CF₃ | CH₃ |
| 1396 | H | | nBu | O | p-C₆H₄—CF₃ | CH₃ |
| 1397 | H | | i-Bu | O | p-C₆H₄—CF₃ | CH₃ |
| 1398 | H | | CH₂—c-Pr | O | p-C₆H₄—CF₃ | CH₃ |
| 1399 | | c-Pr | | O | p-C₆H₄—CF₃ | CH₃ |
| 1400 | c-Pentyl | | H | O | p-C₆H₄—CF₃ | CH₃ |
| 1401 | c-Bu | | | — | Ph | H |
| 1402 | CH₂—c-Bu | | H | — | Ph | H |
| 1403 | 5,5-spiro | | | — | Ph | H |
| | [2.3]hexane | | | | | |
| 1404 | H | | nPr | — | Ph | H |
| 1405 | H | | i-Pr | — | Ph | H |
| 1406 | H | | nBu | — | Ph | H |
| 1407 | H | | i-Bu | — | Ph | H |
| 1408 | H | | CH₂—c-Pr | — | Ph | H |
| 1409 | | c-Pr | | — | Ph | H |
| 1410 | c-Pentyl | | H | — | Ph | H |
| 1411 | c-Bu | | | — | Ph | CF₃ |
| 1412 | CH₂—c-Bu | | H | — | Ph | CF₃ |
| 1413 | 5,5-spiro | | | — | Ph | CF₃ |
| | [2.3]hexane | | | | | |
| 1414 | H | | nPr | — | Ph | CF₃ |
| 1415 | H | | i-Pr | — | Ph | CF₃ |
| 1416 | H | | nBu | — | Ph | CF₃ |
| 1417 | H | | i-Bu | — | Ph | CF₃ |
| 1418 | H | | CH₂—c-Pr | — | Ph | CF₃ |
| 1419 | | c-Pr | | — | Ph | CF₃ |
| 1420 | c-Pentyl | | H | — | Ph | CF₃ |
| 1421 | c-Bu | | | — | Ph | CH₃ |
| 1422 | CH₂—c-Bu | | H | — | Ph | CH₃ |
| 1423 | 5,5-spiro | | | — | Ph | CH₃ |
| | [2.3]hexane | | | | | |
| 1424 | H | | nPr | — | Ph | CH₃ |
| 1425 | H | | i-Pr | — | Ph | CH₃ |
| 1426 | H | | nBu | — | Ph | CH₃ |
| 1427 | H | | i-Bu | — | Ph | CH₃ |
| 1428 | H | | CH₂—c-Pr | — | Ph | CH₃ |
| 1429 | | c-Pr | | — | Ph | CH₃ |
| 1430 | c-Pentyl | | H | — | Ph | CH₃ |
| 1431 | c-Bu | | | — | p-C₆H₄—F | H |
| 1432 | CH₂—c-Bu | | H | — | p-C₆H₄—F | H |
| 1433 | 5,5-spiro | | | — | p-C₆H₄—F | H |
| | [2.3]hexane | | | | | |
| 1434 | H | | nPr | — | p-C₆H₄—F | H |
| 1435 | H | | i-Pr | — | p-C₆H₄—F | H |
| 1436 | H | | nBu | — | p-C₆H₄—F | H |
| 1437 | H | | i-Bu | — | p-C₆H₄—F | H |
| 1438 | H | | CH₂—c-Pr | — | p-C₆H₄—F | H |
| 1439 | | c-Pr | | — | p-C₆H₄—F | H |
| 1440 | c-Pentyl | | H | — | p-C₆H₄—F | H |
| 1441 | c-Bu | | | — | p-C₆H₄—F | CF₃ |
| 1442 | CH₂—c-Bu | | H | — | p-C₆H₄—F | CF₃ |
| 1443 | 5,5-spiro | | | — | p-C₆H₄—F | CF₃ |
| | [2.3]hexane | | | | | |
| 1444 | H | | nPr | — | p-C₆H₄—F | CF₃ |
| 1445 | H | | i-Pr | — | p-C₆H₄—F | CF₃ |
| 1446 | H | | nBu | — | p-C₆H₄—F | CF₃ |
| 1447 | H | | i-Bu | — | p-C₆H₄—F | CF₃ |
| 1448 | H | | CH₂—c-Pr | — | p-C₆H₄—F | CF₃ |
| 1449 | | c-Pr | | — | p-C₆H₄—F | CF₃ |
| 1450 | c-Pentyl | | H | — | p-C₆H₄—F | CF₃ |
| 1451 | c-Bu | | | — | p-C₆H₄—F | CH₃ |
| 1452 | CH₂—c-Bu | | H | — | p-C₆H₄—F | CH₃ |
| 1453 | 5,5-spiro | | | — | p-C₆H₄—F | CH₃ |
| | [2.3]hexane | | | | | |
| 1454 | H | | nPr | — | p-C₆H₄—F | CH₃ |
| 1455 | H | | i-Pr | — | p-C₆H₄—F | CH₃ |
| 1456 | H | | nBu | — | p-C₆H₄—F | CH₃ |
| 1457 | H | | i-Bu | — | p-C₆H₄—F | CH₃ |
| 1458 | H | | CH₂—c-Pr | — | p-C₆H₄—F | CH₃ |
| 1459 | | c-Pr | | — | p-C₆H₄—F | CH₃ |
| 1460 | c-Pentyl | | H | — | p-C₆H₄—F | CH₃ |
| 1461 | c-Bu | | | — | p-C₆H₄—Cl | H |
| 1462 | CH₂—c-Bu | | H | — | p-C₆H₄—Cl | H |
| 1463 | 5,5-spiro | | | — | p-C₆H₄—Cl | H |
| | [2.3]hexane | | | | | |
| 1464 | H | | nPr | — | p-C₆H₄—Cl | H |
| 1465 | H | | i-Pr | — | p-C₆H₄—Cl | H |
| 1466 | H | | nBu | — | p-C₆H₄—Cl | H |
| 1467 | H | | i-Bu | — | p-C₆H₄—Cl | H |
| 1468 | H | | CH₂—c-Pr | — | p-C₆H₄—Cl | H |
| 1469 | | c-Pr | | — | p-C₆H₄—Cl | H |
| 1470 | c-Pentyl | | H | — | p-C₆H₄—Cl | H |
| 1471 | c-Bu | | | — | p-C₆H₄—Cl | CF₃ |
| 1472 | CH₂—c-Bu | | H | — | p-C₆H₄—Cl | CF₃ |
| 1473 | 5,5-spiro | | | — | p-C₆H₄—Cl | CF₃ |
| | [2.3]hexane | | | | | |
| 1474 | H | | nPr | — | p-C₆H₄—Cl | CF₃ |
| 1475 | H | | i-Pr | — | p-C₆H₄—Cl | CF₃ |
| 1476 | H | | nBu | — | p-C₆H₄—Cl | CF₃ |
| 1477 | H | | i-Bu | — | p-C₆H₄—Cl | CF₃ |
| 1478 | H | | CH₂—c-Pr | — | p-C₆H₄—Cl | CF₃ |
| 1479 | | c-Pr | | — | p-C₆H₄—Cl | CF₃ |
| 1480 | c-Pentyl | | H | — | p-C₆H₄—Cl | CF₃ |
| 1481 | c-Bu | | | — | p-C₆H₄—Cl | CH₃ |
| 1482 | CH₂—c-Bu | | H | — | p-C₆H₄—Cl | CH₃ |
| 1483 | 5,5-spiro | | | — | p-C₆H₄—Cl | CH₃ |
| | [2.3]hexane | | | | | |
| 1484 | H | | nPr | — | p-C₆H₄—Cl | CH₃ |
| 1485 | H | | i-Pr | — | p-C₆H₄—Cl | CH₃ |
| 1486 | H | | nBu | — | p-C₆H₄—Cl | CH₃ |
| 1487 | H | | i-Bu | — | p-C₆H₄—Cl | CH₃ |
| 1488 | H | | CH₂—c-Pr | — | p-C₆H₄—Cl | CH₃ |
| 1489 | | c-Pr | | — | p-C₆H₄—Cl | CH₃ |
| 1490 | c-Pentyl | | H | — | p-C₆H₄—Cl | CH₃ |
| 1491 | c-Bu | | | — | p-C₆H₄—CF₃ | H |
| 1492 | CH₂—c-Bu | | H | — | p-C₆H₄—CF₃ | H |
| 1493 | 5,5-spiro | | | — | p-C₆H₄—CF₃ | H |
| | [2.3]hexane | | | | | |
| 1494 | H | | nPr | — | p-C₆H₄—CF₃ | H |
| 1495 | H | | i-Pr | — | p-C₆H₄—CF₃ | H |
| 1496 | H | | nBu | — | p-C₆H₄—CF₃ | H |
| 1497 | H | | i-Bu | — | p-C₆H₄—CF₃ | H |
| 1498 | H | | CH₂—c-Pr | — | p-C₆H₄—CF₃ | H |
| 1499 | | c-Pr | | — | p-C₆H₄—CF₃ | H |
| 1500 | c-Pentyl | | H | — | p-C₆H₄—CF₃ | H |
| 1501 | c-Bu | | | — | p-C₆H₄—CF₃ | CF₃ |
| 1502 | CH₂—c-Bu | | H | — | p-C₆H₄—CF₃ | CF₃ |
| 1503 | 5,5-spiro | | | — | p-C₆H₄—CF₃ | CF₃ |
| | [2.3]hexane | | | | | |
| 1504 | H | | nPr | — | p-C₆H₄—CF₃ | CF₃ |
| 1505 | H | | i-Pr | — | p-C₆H₄—CF₃ | CF₃ |
| 1506 | H | | nBu | — | p-C₆H₄—CF₃ | CF₃ |
| 1507 | H | | i-Bu | — | p-C₆H₄—CF₃ | CF₃ |
| 1508 | H | | CH₂—c-Pr | — | p-C₆H₄—CF₃ | CF₃ |
| 1509 | | c-Pr | | — | p-C₆H₄—CF₃ | CF₃ |
| 1510 | c-Pentyl | | H | — | p-C₆H₄—CF₃ | CF₃ |
| 1511 | c-Bu | | | — | p-C₆H₄—CF₃ | CH₃ |
| 1512 | CH₂—c-Bu | | H | — | p-C₆H₄—CF₃ | CH₃ |
| 1513 | 5,5-spiro | | | — | p-C₆H₄—CF₃ | CH₃ |
| | [2.3]hexane | | | | | |
| 1514 | H | | nPr | — | p-C₆H₄—CF₃ | CH₃ |
| 1515 | H | | i-Pr | — | p-C₆H₄—CF₃ | CH₃ |
| 1516 | H | | nBu | — | p-C₆H₄—CF₃ | CH₃ |
| 1517 | H | | i-Bu | — | p-C₆H₄—CF₃ | CH₃ |
| 1518 | H | | CH₂—c-Pr | — | p-C₆H₄—CF₃ | CH₃ |
| 1519 | | c-Pr | | — | p-C₆H₄—CF₃ | CH₃ |
| 1520 | c-Pentyl | | H | — | p-C₆H₄—CF₃ | CH₃ |

A compound of formula (IX) where

TABLE 6
(IX)

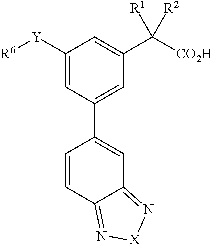

| Cpd # | R¹ | | R² | Y | R⁶ | X |
|---|---|---|---|---|---|---|
| 1521 | c-Bu | | | O | —CH₂-cyclopropyl | O |
| 1522 | CH₂—c-Bu | | H | O | —CH₂-cyclopropyl | O |
| 1523 | 5,5-spiro | | | O | —CH₂-cyclopropyl | O |
| | [2.3]hexane | | | | | |
| 1524 | H | | nPr | O | —CH₂-cyclopropyl | O |
| 1525 | H | | i-Pr | O | —CH₂-cyclopropyl | O |
| 1526 | H | | nBu | O | —CH₂-cyclopropyl | O |
| 1527 | H | | i-Bu | O | —CH₂-cyclopropyl | O |
| 1528 | H | | CH₂—c-Pr | O | —CH₂-cyclopropyl | O |
| 1529 | | c-Pr | | O | —CH₂-cyclopropyl | O |
| 1530 | c-Pentyl | | H | O | —CH₂-cyclopropyl | O |
| 1531 | c-Bu | | | O | —CH₂-cyclopropyl | S |
| 1532 | CH₂—c-Bu | | H | O | —CH₂-cyclopropyl | S |
| 1533 | 5,5-spiro | | | O | —CH₂-cyclopropyl | S |
| | [2.3]hexane | | | | | |
| 1534 | H | | nPr | O | —CH₂-cyclopropyl | S |
| 1535 | H | | i-Pr | O | —CH₂-cyclopropyl | S |
| 1536 | H | | nBu | O | —CH₂-cyclopropyl | S |
| 1537 | H | | i-Bu | O | —CH₂-cyclopropyl | S |
| 1538 | H | | CH₂—c-Pr | O | —CH₂-cyclopropyl | S |
| 1539 | | c-Pr | | O | —CH₂-cyclopropyl | S |
| 1540 | c-Pentyl | | H | O | —CH₂-cyclopropyl | S |
| 1541 | c-Bu | | | O | —CH₂—p-C₆H₄—F | O |
| 1542 | CH₂—c-Bu | | H | O | —CH₂—p-C₆H₄—F | O |
| 1543 | 5,5-spiro | | | O | —CH₂—p-C₆H₄—F | O |
| | [2.3]hexane | | | | | |
| 1544 | H | | nPr | O | —CH₂—p-C₆H₄—F | O |
| 1545 | H | | i-Pr | O | —CH₂—p-C₆H₄—F | O |
| 1546 | H | | nBu | O | —CH₂—p-C₆H₄—F | O |
| 1547 | H | | i-Bu | O | —CH₂—p-C₆H₄—F | O |
| 1548 | H | | CH₂—c-Pr | O | —CH₂—p-C₆H₄—F | O |
| 1549 | | c-Pr | | O | —CH₂—p-C₆H₄—F | O |
| 1550 | c-Pentyl | | H | O | —CH₂—p-C₆H₄—F | O |
| 1551 | c-Bu | | | O | —CH₂—p-C₆H₄—F | S |
| 1552 | CH₂—c-Bu | | H | O | —CH₂—p-C₆H₄—F | S |
| 1553 | 5,5-spiro | | | O | —CH₂—p-C₆H₄—F | S |
| | [2.3]hexane | | | | | |
| 1554 | H | | nPr | O | —CH₂—p-C₆H₄—F | S |
| 1555 | H | | i-Pr | O | —CH₂—p-C₆H₄—F | S |
| 1556 | H | | nBu | O | —CH₂—p-C₆H₄—F | S |
| 1557 | H | | i-Bu | O | —CH₂—p-C₆H₄—F | S |
| 1558 | H | | CH₂—c-Pr | O | —CH₂—p-C₆H₄—F | S |
| 1559 | | c-Pr | | O | —CH₂—p-C₆H₄—F | S |
| 1560 | c-Pentyl | | H | O | —CH₂—p-C₆H₄—F | S |
| 1561 | c-Bu | | | O | —CH₂—p-C₆H₄—Cl | O |
| 1562 | CH₂—c-Bu | | H | O | —CH₂—p-C₆H₄—Cl | O |
| 1563 | 5,5-spiro | | | O | —CH₂—p-C₆H₄—Cl | O |
| | [2.3]hexane | | | | | |
| 1564 | H | | nPr | O | —CH₂—p-C₆H₄—Cl | O |
| 1565 | H | | i-Pr | O | —CH₂—p-C₆H₄—Cl | O |
| 1566 | H | | nBu | O | —CH₂—p-C₆H₄—Cl | O |
| 1567 | H | | i-Bu | O | —CH₂—p-C₆H₄—Cl | O |
| 1568 | H | | CH₂—c-Pr | O | —CH₂—p-C₆H₄—Cl | O |
| 1569 | | c-Pr | | O | —CH₂—p-C₆H₄—Cl | O |
| 1570 | c-Pentyl | | H | O | —CH₂—p-C₆H₄—Cl | O |
| 1571 | c-Bu | | | O | —CH₂—p-C₆H₄—Cl | S |
| 1572 | CH₂—c-Bu | | H | O | —CH₂—p-C₆H₄—Cl | S |
| 1573 | 5,5-spiro | | | O | —CH₂—p-C₆H₄—Cl | S |
| | [2.3]hexane | | | | | |
| 1574 | H | | nPr | O | —CH₂—p-C₆H₄—Cl | S |
| 1575 | H | | i-Pr | O | —CH₂—p-C₆H₄—Cl | S |
| 1576 | H | | nBu | O | —CH₂—p-C₆H₄—Cl | S |
| 1577 | H | | i-Bu | O | —CH₂—p-C₆H₄—Cl | S |
| 1578 | H | | CH₂—c-Pr | O | —CH₂—p-C₆H₄—Cl | S |
| 1579 | | c-Pr | | O | —CH₂—p-C₆H₄—Cl | S |
| 1580 | c-Pentyl | | H | O | —CH₂—p-C₆H₄—Cl | S |
| 1581 | c-Bu | | | O | —CH₂—p-C₆H₄—CF₃ | O |
| 1582 | CH₂—c-Bu | | H | O | —CH₂—p-C₆H₄—CF₃ | O |
| 1583 | 5,5-spiro | | | O | —CH₂—p-C₆H₄—CF₃ | O |
| | [2.3]hexane | | | | | |
| 1584 | H | | nPr | O | —CH₂—p-C₆H₄—CF₃ | O |
| 1585 | H | | i-Pr | O | —CH₂—p-C₆H₄—CF₃ | O |
| 1586 | H | | nBu | O | —CH₂—p-C₆H₄—CF₃ | O |
| 1587 | H | | i-Bu | O | —CH₂—p-C₆H₄—CF₃ | O |
| 1588 | H | | CH₂—c-Pr | O | —CH₂—p-C₆H₄—CF₃ | O |
| 1589 | | c-Pr | | O | —CH₂—p-C₆H₄—CF₃ | O |
| 1590 | c-Pentyl | | H | O | —CH₂—p-C₆H₄—CF₃ | O |
| 1591 | c-Bu | | | O | —CH₂—p-C₆H₄—CF₃ | S |
| 1592 | CH₂—c-Bu | | H | O | —CH₂—p-C₆H₄—CF₃ | S |
| 1593 | 5,5-spiro | | | O | —CH₂—p-C₆H₄—CF₃ | S |
| | [2.3]hexane | | | | | |
| 1594 | H | | nPr | O | —CH₂—p-C₆H₄—CF₃ | S |
| 1595 | H | | i-Pr | O | —CH₂—p-C₆H₄—CF₃ | S |
| 1596 | H | | nBu | O | —CH₂—p-C₆H₄—CF₃ | S |
| 1597 | H | | i-Bu | O | —CH₂—p-C₆H₄—CF₃ | S |
| 1598 | H | | CH₂—c-Pr | O | —CH₂—p-C₆H₄—CF₃ | S |
| 1599 | | c-Pr | | O | —CH₂—p-C₆H₄—CF₃ | S |
| 1600 | c-Pentyl | | H | O | —CH₂—p-C₆H₄—CF₃ | S |
| 1601 | c-Bu | | | O | Et | O |
| 1602 | CH₂—c-Bu | | H | O | Et | O |
| 1603 | 5,5-spiro | | | O | Et | O |
| | [2.3]hexane | | | | | |
| 1604 | H | | nPr | O | Et | O |
| 1605 | H | | i-Pr | O | Et | O |
| 1606 | H | | nBu | O | Et | O |
| 1607 | H | | i-Bu | O | Et | O |
| 1608 | H | | CH₂—c-Pr | O | Et | O |
| 1609 | | c-Pr | | O | Et | O |
| 1610 | c-Pentyl | | H | O | Et | O |
| 1611 | c-Bu | | | O | Et | S |
| 1612 | CH₂—c-Bu | | H | O | Et | S |
| 1613 | 5,5-spiro | | | O | Et | S |
| | [2.3]hexane | | | | | |
| 1614 | H | | nPr | O | Et | S |
| 1615 | H | | i-Pr | O | Et | S |
| 1616 | H | | nBu | O | Et | S |
| 1617 | H | | i-Bu | O | Et | S |
| 1618 | H | | CH₂—c-Pr | O | Et | S |
| 1619 | | c-Pr | | O | Et | S |
| 1620 | c-Pentyl | | H | O | Et | S |
| 1621 | c-Bu | | | O | CH₂CF₃ | O |
| 1622 | CH₂—c-Bu | | H | O | CH₂CF₃ | O |
| 1623 | 5,5-spiro | | | O | CH₂CF₃ | O |
| | [2.3]hexane | | | | | |
| 1624 | H | | nPr | O | CH₂CF₃ | O |
| 1625 | H | | i-Pr | O | CH₂CF₃ | O |
| 1626 | H | | nBu | O | CH₂CF₃ | O |
| 1627 | H | | i-Bu | O | CH₂CF₃ | O |
| 1628 | H | | CH₂—c-Pr | O | CH₂CF₃ | O |
| 1629 | | c-Pr | | O | CH₂CF₃ | O |
| 1630 | c-Pentyl | | H | O | CH₂CF₃ | O |
| 1631 | c-Bu | | | O | CH₂CF₃ | S |
| 1632 | CH₂—c-Bu | | H | O | CH₂CF₃ | S |
| 1633 | 5,5-spiro | | | O | CH₂CF₃ | S |
| | [2.3]hexane | | | | | |
| 1634 | H | | nPr | O | CH₂CF₃ | S |
| 1635 | H | | i-Pr | O | CH₂CF₃ | S |
| 1636 | H | | nBu | O | CH₂CF₃ | S |
| 1637 | H | | i-Bu | O | CH₂CF₃ | S |
| 1638 | H | | CH₂—c-Pr | O | CH₂CF₃ | S |
| 1639 | | c-Pr | | O | CH₂CF₃ | S |
| 1640 | c-Pentyl | | H | O | CH₂CF₃ | S |
| 1641 | c-Bu | | | O | CH₂CH₂OMe | O |
| 1642 | CH₂—c-Bu | | H | O | CH₂CH₂OMe | O |
| 1643 | 5,5-spiro | | | O | CH₂CH₂OMe | O |
| | [2.3]hexane | | | | | |
| 1644 | H | | nPr | O | CH₂CH₂OMe | O |
| 1645 | H | | i-Pr | O | CH₂CH₂OMe | O |
| 1646 | H | | nBu | O | CH₂CH₂OMe | O |
| 1647 | H | | i-Bu | O | CH₂CH₂OMe | O |
| 1648 | H | | CH₂—c-Pr | O | CH₂CH₂OMe | O |
| 1649 | | c-Pr | | O | CH₂CH₂OMe | O |
| 1650 | c-Pentyl | | H | O | CH₂CH₂OMe | O |
| 1651 | c-Bu | | | O | CH₂CH₂OMe | S |
| 1652 | CH₂—c-Bu | | H | O | CH₂CH₂OMe | S |
| 1653 | 5,5-spiro | | | O | CH₂CH₂OMe | S |
| | [2.3]hexane | | | | | |
| 1654 | H | | nPr | O | CH₂CH₂OMe | S |
| 1655 | H | | i-Pr | O | CH₂CH₂OMe | S |
| 1656 | H | | nBu | O | CH₂CH₂OMe | S |
| 1657 | H | | i-Bu | O | CH₂CH₂OMe | S |
| 1658 | H | | CH₂—c-Pr | O | CH₂CH₂OMe | S |
| 1659 | | c-Pr | | O | CH₂CH₂OMe | S |
| 1660 | c-Pentyl | | H | O | CH₂CH₂OMe | S |
| 1661 | c-Bu | | | O | Ph | O |
| 1662 | CH₂—c-Bu | | H | O | Ph | O |
| 1663 | 5,5-spiro | | | O | Ph | O |
| | [2.3]hexane | | | | | |
| 1664 | H | | nPr | O | Ph | O |
| 1665 | H | | i-Pr | O | Ph | O |
| 1666 | H | | nBu | O | Ph | O |
| 1667 | H | | i-Bu | O | Ph | O |
| 1668 | H | | CH₂—c-Pr | O | Ph | O |
| 1669 | | c-Pr | | O | Ph | O |
| 1670 | c-Pentyl | | H | O | Ph | O |
| 1671 | c-Bu | | | O | Ph | S |
| 1672 | CH₂—c-Bu | | H | O | Ph | S |
| 1673 | 5,5-spiro | | | O | Ph | S |
| | [2.3]hexane | | | | | |
| 1674 | H | | nPr | O | Ph | S |
| 1675 | H | | i-Pr | O | Ph | S |
| 1676 | H | | nBu | O | Ph | S |
| 1677 | H | | i-Bu | O | Ph | S |
| 1678 | H | | CH₂—c-Pr | O | Ph | S |
| 1679 | | c-Pr | | O | Ph | S |
| 1680 | c-Pentyl | | H | O | Ph | S |
| 1681 | c-Bu | | | O | p-C₆H₄—F | O |
| 1682 | CH₂—c-Bu | | H | O | p-C₆H₄—F | O |
| 1683 | 5,5-spiro | | | O | p-C₆H₄—F | O |
| | [2.3]hexane | | | | | |
| 1684 | H | | nPr | O | p-C₆H₄—F | O |
| 1685 | H | | i-Pr | O | p-C₆H₄—F | O |
| 1686 | H | | nBu | O | p-C₆H₄—F | O |
| 1687 | H | | i-Bu | O | p-C₆H₄—F | O |
| 1688 | H | | CH₂—c-Pr | O | p-C₆H₄—F | O |
| 1689 | | c-Pr | | O | p-C₆H₄—F | O |
| 1690 | c-Pentyl | | H | O | p-C₆H₄—F | O |
| 1691 | c-Bu | | | O | p-C₆H₄—F | S |
| 1692 | CH₂—c-Bu | | H | O | p-C₆H₄—F | S |
| 1693 | 5,5-spiro | | | O | p-C₆H₄—F | S |
| | [2.3]hexane | | | | | |
| 1694 | H | | nPr | O | p-C₆H₄—F | S |
| 1695 | H | | i-Pr | O | p-C₆H₄—F | S |
| 1696 | H | | nBu | O | p-C₆H₄—F | S |
| 1697 | H | | i-Bu | O | p-C₆H₄—F | S |
| 1698 | H | | CH₂—c-Pr | O | p-C₆H₄—F | S |
| 1699 | | c-Pr | | O | p-C₆H₄—F | S |
| 1700 | c-Pentyl | | H | O | p-C₆H₄—F | S |
| 1701 | c-Bu | | | O | p-C₆H₄—Cl | O |
| 1702 | CH₂—c-Bu | | H | O | p-C₆H₄—Cl | O |
| 1703 | 5,5-spiro | | | O | p-C₆H₄—Cl | O |
| | [2.3]hexane | | | | | |
| 1704 | H | | nPr | O | p-C₆H₄—Cl | O |
| 1705 | H | | i-Pr | O | p-C₆H₄—Cl | O |
| 1706 | H | | nBu | O | p-C₆H₄—Cl | O |
| 1707 | H | | i-Bu | O | p-C₆H₄—Cl | O |
| 1708 | H | | CH₂—c-Pr | O | p-C₆H₄—Cl | O |
| 1709 | | c-Pr | | O | p-C₆H₄—Cl | O |
| 1710 | c-Pentyl | | H | O | p-C₆H₄—Cl | O |
| 1711 | c-Bu | | | O | p-C₆H₄—Cl | S |
| 1712 | CH₂—c-Bu | | H | O | p-C₆H₄—Cl | S |
| 1713 | 5,5-spiro | | | O | p-C₆H₄—Cl | S |
| | [2.3]hexane | | | | | |
| 1714 | H | | nPr | O | p-C₆H₄—Cl | S |
| 1715 | H | | i-Pr | O | p-C₆H₄—Cl | S |
| 1716 | H | | nBu | O | p-C₆H₄—Cl | S |
| 1717 | H | | i-Bu | O | p-C₆H₄—Cl | S |
| 1718 | H | | CH₂—c-Pr | O | p-C₆H₄—Cl | S |
| 1719 | | c-Pr | | O | p-C₆H₄—Cl | S |
| 1720 | c-Pentyl | | H | O | p-C₆H₄—Cl | S |
| 1721 | c-Bu | | | O | p-C₆H₄—CF₃ | O |
| 1722 | CH₂—c-Bu | | H | O | p-C₆H₄—CF₃ | O |
| 1723 | 5,5-spiro | | | O | p-C₆H₄—CF₃ | O |
| | [2.3]hexane | | | | | |
| 1724 | H | | nPr | O | p-C₆H₄—CF₃ | O |
| 1725 | H | | i-Pr | O | p-C₆H₄—CF₃ | O |
| 1726 | H | | nBu | O | p-C₆H₄—CF₃ | O |
| 1727 | H | | i-Bu | O | p-C₆H₄—CF₃ | O |
| 1728 | H | | CH₂—c-Pr | O | p-C₆H₄—CF₃ | O |
| 1729 | | c-Pr | | O | p-C₆H₄—CF₃ | O |
| 1730 | c-Pentyl | | H | O | p-C₆H₄—CF₃ | O |
| 1731 | c-Bu | | | O | p-C₆H₄—CF₃ | S |
| 1732 | CH₂—c-Bu | | H | O | p-C₆H₄—CF₃ | S |
| 1733 | 5,5-spiro | | | O | p-C₆H₄—CF₃ | S |
| | [2.3]hexane | | | | | |
| 1734 | H | | nPr | O | p-C₆H₄—CF₃ | S |
| 1735 | H | | i-Pr | O | p-C₆H₄—CF₃ | S |
| 1736 | H | | nBu | O | p-C₆H₄—CF₃ | S |
| 1737 | H | | i-Bu | O | p-C₆H₄—CF₃ | S |
| 1738 | H | | CH₂—c-Pr | O | p-C₆H₄—CF₃ | S |
| 1739 | | c-Pr | | O | p-C₆H₄—CF₃ | S |
| 1740 | c-Pentyl | | H | O | p-C₆H₄—CF₃ | S |
| 1741 | c-Bu | | | — | Ph | O |
| 1742 | CH₂—c-Bu | | H | — | Ph | O |
| 1743 | 5,5-spiro | | | — | Ph | O |
| | [2.3]hexane | | | | | |
| 1744 | H | | nPr | — | Ph | O |
| 1745 | H | | i-Pr | — | Ph | O |
| 1746 | H | | nBu | — | Ph | O |
| 1747 | H | | i-Bu | — | Ph | O |
| 1748 | H | | CH₂—c-Pr | — | Ph | O |
| 1749 | | c-Pr | | — | Ph | O |
| 1750 | c-Pentyl | | H | — | Ph | O |
| 1751 | c-Bu | | | — | Ph | S |
| 1752 | CH₂—c-Bu | | H | — | Ph | S |
| 1753 | 5,5-spiro | | | — | Ph | S |
| | [2.3]hexane | | | | | |
| 1754 | H | | nPr | — | Ph | S |
| 1755 | H | | i-Pr | — | Ph | S |
| 1756 | H | | nBu | — | Ph | S |
| 1757 | H | | i-Bu | — | Ph | S |
| 1758 | H | | CH₂—c-Pr | — | Ph | S |
| 1759 | | c-Pr | | — | Ph | S |
| 1760 | c-Pentyl | | H | — | Ph | S |
| 1761 | c-Bu | | | — | p-C₆H₄—F | O |
| 1762 | CH₂—c-Bu | | H | — | p-C₆H₄—F | O |
| 1763 | 5,5-spiro | | | — | p-C₆H₄—F | O |
| | [2.3]hexane | | | | | |
| 1764 | H | | nPr | — | p-C₆H₄—F | O |
| 1765 | H | | i-Pr | — | p-C₆H₄—F | O |
| 1766 | H | | nBu | — | p-C₆H₄—F | O |
| 1767 | H | | i-Bu | — | p-C₆H₄—F | O |
| 1768 | H | | CH₂—c-Pr | — | p-C₆H₄—F | O |
| 1769 | | c-Pr | | — | p-C₆H₄—F | O |
| 1770 | c-Pentyl | | H | — | p-C₆H₄—F | O |
| 1771 | c-Bu | | | — | p-C₆H₄—F | S |
| 1772 | CH₂—c-Bu | | H | — | p-C₆H₄—F | S |
| 1773 | 5,5-spiro | | | — | p-C₆H₄—F | S |
| | [2.3]hexane | | | | | |
| 1774 | H | | nPr | — | p-C₆H₄—F | S |
| 1775 | H | | i-Pr | — | p-C₆H₄—F | S |
| 1776 | H | | nBu | — | p-C₆H₄—F | S |
| 1777 | H | | i-Bu | — | p-C₆H₄—F | S |
| 1778 | H | | CH₂—c-Pr | — | p-C₆H₄—F | S |
| 1779 | | c-Pr | | — | p-C₆H₄—F | S |
| 1780 | c-Pentyl | | H | — | p-C₆H₄—F | S |
| 1781 | c-Bu | | | — | p-C₆H₄—Cl | O |
| 1782 | CH₂—c-Bu | | H | — | p-C₆H₄—Cl | O |
| 1783 | 5,5-spiro | | | — | p-C₆H₄—Cl | O |
| | [2.3]hexane | | | | | |
| 1784 | H | | nPr | — | p-C₆H₄—Cl | O |
| 1785 | H | | i-Pr | — | p-C₆H₄—Cl | O |
| 1786 | H | | nBu | — | p-C₆H₄—Cl | O |
| 1787 | H | | i-Bu | — | p-C₆H₄—Cl | O |
| 1788 | H | | CH₂—c-Pr | — | p-C₆H₄—Cl | O |
| 1789 | | c-Pr | | — | p-C₆H₄—Cl | O |
| 1790 | c-Pentyl | | H | — | p-C₆H₄—Cl | O |
| 1791 | c-Bu | | | — | p-C₆H₄—Cl | S |
| 1792 | CH₂—c-Bu | | H | — | p-C₆H₄—Cl | S |
| 1793 | 5,5-spiro | | | — | p-C₆H₄—Cl | S |
| | [2.3]hexane | | | | | |
| 1794 | H | | nPr | — | p-C₆H₄—Cl | S |
| 1795 | H | | i-Pr | — | p-C₆H₄—Cl | S |
| 1796 | H | | nBu | — | p-C₆H₄—Cl | S |
| 1797 | H | | i-Bu | — | p-C₆H₄—Cl | S |
| 1798 | H | | CH₂—c-Pr | — | p-C₆H₄—Cl | S |
| 1799 | | c-Pr | | — | p-C₆H₄—Cl | S |
| 1800 | c-Pentyl | | H | — | p-C₆H₄—Cl | S |
| 1801 | c-Bu | | | — | p-C₆H₄—CF₃ | O |
| 1802 | CH₂—c-Bu | | H | — | p-C₆H₄—CF₃ | O |
| 1803 | 5,5-spiro | | | — | p-C₆H₄—CF₃ | O |
| | [2.3]hexane | | | | | |
| 1804 | H | | nPr | — | p-C₆H₄—CF₃ | O |
| 1805 | H | | i-Pr | — | p-C₆H₄—CF₃ | O |
| 1806 | H | | nBu | — | p-C₆H₄—CF₃ | O |
| 1807 | H | | i-Bu | — | p-C₆H₄—CF₃ | O |
| 1808 | H | | CH₂—c-Pr | — | p-C₆H₄—CF₃ | O |
| 1809 | | c-Pr | | — | p-C₆H₄—CF₃ | O |
| 1810 | c-Pentyl | | H | — | p-C₆H₄—CF₃ | O |
| 1811 | c-Bu | | | — | p-C₆H₄—CF₃ | S |
| 1812 | CH₂—c-Bu | | H | — | p-C₆H₄—CF₃ | S |
| 1813 | 5,5-spiro | | | — | p-C₆H₄—CF₃ | S |
| | [2.3]hexane | | | | | |
| 1814 | H | | nPr | — | p-C₆H₄—CF₃ | S |
| 1815 | H | | i-Pr | — | p-C₆H₄—CF₃ | S |
| 1816 | H | | nBu | — | p-C₆H₄—CF₃ | S |
| 1817 | H | | i-Bu | — | p-C₆H₄—CF₃ | S |
| 1818 | H | | CH₂—c-Pr | — | p-C₆H₄—CF₃ | S |
| 1819 | | c-Pr | | — | p-C₆H₄—CF₃ | S |
| 1820 | c-Pentyl | | H | — | p-C₆H₄—CF₃ | S |

A compound of formula (X) where

TABLE 7
(X)

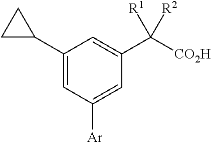

| Cpd # | R¹ | R² | Ar |
|---|---|---|---|
| 1900 | c-Bu | | p-C₆H₄—CF₃ |
| 1901 | CH₂—c-Bu | H | p-C₆H₄—CF₃ |
| 1902 | 5,5-spiro | | p-C₆H₄—CF₃ |
| | [2.3]hexane | | |
| 1903 | H | nPr | p-C₆H₄—CF₃ |
| 1904 | H | i-Pr | p-C₆H₄—CF₃ |
| 1905 | H | nBu | p-C₆H₄—CF₃ |
| 1906 | H | i-Bu | p-C₆H₄—CF₃ |
| 1907 | H | CH₂—c-Pr | p-C₆H₄—CF₃ |
| 1908 | c-Pr | — | p-C₆H₄—CF₃ |
| 1909 | c-Pentyl | H | p-C₆H₄—CF₃ |
| 1910 | c-Bu | | benzo[c][1,2,5]thiadiazol-5-y1 |
| 1911 | CH₂—c-Bu | H | benzo[c][1,2,5]thiadiazol-5-y1 |
| 1912 | 5,5-spiro | | benzo[c][1,2,5]thiadiazol-5-y1 |
| | [2.3]hexane | | |
| 1913 | H | nPr | benzo[c][1,2,5]thiadiazol-5-y1 |
| 1914 | H | i-Pr | benzo[c][1,2,5]thiadiazol-5-y1 |
| 1915 | H | nBu | benzo[c][1,2,5]thiadiazol-5-y1 |
| 1916 | H | i-Bu | benzo[c][1,2,5]thiadiazol-5-y1 |
| 1917 | H | CH₂—c-Pr | benzo[c][1,2,5]thiadiazol-5-y1 |
| 1918 | c-Pr | — | benzo[c][1,2,5]thiadiazol-5-y1 |
| 1919 | c-Pentyl | H | benzo[c][1,2,5]thiadiazol-5-y1 |
| 1920 | c-Bu | | benzo[c][1,2,5]oxadiazol-5-y1 |
| 1921 | CH₂—c-Bu | H | benzo[c][1,2,5]oxadiazol-5-y1 |
| 1922 | 5,5-spiro | | benzo[c][1,2,5]oxadiazol-5-y1 |
| | [2.3]hexane | | |
| 1923 | H | nPr | benzo[c][1,2,5]oxadiazol-5-y1 |
| 1924 | H | i-Pr | benzo[c][1,2,5]oxadiazol-5-y1 |
| 1925 | H | nBu | benzo[c][1,2,5]oxadiazol-5-y1 |
| 1926 | H | i-Bu | benzo[c][1,2,5]oxadiazol-5-y1 |
| 1927 | H | CH₂—c-Pr | benzo[c][1,2,5]oxadiazol-5-y1 |
| 1928 | c-Pr | — | benzo[c][1,2,5]oxadiazol-5-y1 |
| 1929 | c-Pentyl | H | benzo[c][1,2,5]oxadiazol-5-y1 |

A compound of formula (XI) where

(XI)

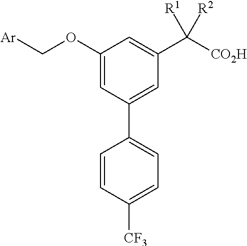

| Cpd # | R¹ | R² | Ar |
|---|---|---|---|
| 1930 | c-Bu | | benzo[c][1,2,5]thiadiazol-5-y1 |
| 1931 | CH₂—c-Bu | H | benzo[c][1,2,5]thiadiazol-5-y1 |
| 1932 | 5,5-spiro | | benzo[c][1,2,5]thiadiazol-5-y1 |
| | [2.3]hexane | | |
| 1933 | H | nPr | benzo[c][1,2,5]thiadiazol-5-y1 |
| 1934 | H | i-Pr | benzo[c][1,2,5]thiadiazol-5-y1 |
| 1935 | H | nBu | benzo[c][1,2,5]thiadiazol-5-y1 |
| 1936 | H | i-Bu | benzo[c][1,2,5]thiadiazol-5-y1 |
| 1937 | H | CH₂—c-Pr | benzo[c][1,2,5]thiadiazol-5-y1 |
| 1938 | c-Pr | — | benzo[c][1,2,5]thiadiazol-5-y1 |
| 1939 | c-Pentyl | H | benzo[c][1,2,5]thiadiazol-5-y1 |
| 1940 | c-Bu | | benzo[c][1,2,5]oxadiazol-5-y1 |
| 1941 | CH₂—c-Bu | H | benzo[c][1,2,5]oxadiazol-5-y1 |
| 1942 | 5,5-spiro | | benzo[c][1,2,5]oxadiazol-5-y1 |
| | [2.3]hexane | | |
| 1943 | H | nPr | benzo[c][1,2,5]oxadiazol-5-y1 |
| 1944 | H | i-Pr | benzo[c][1,2,5]oxadiazol-5-y1 |
| 1945 | H | nBu | benzo[c][1,2,5]oxadiazol-5-y1 |
| 1946 | H | i-Bu | benzo[c][1,2,5]oxadiazol-5-y1 |
| 1947 | H | CH₂—c-Pr | benzo[c][1,2,5]oxadiazol-5-y1 |
| 1948 | c-Pr | — | benzo[c][1,2,5]oxadiazol-5-y1 |
| 1949 | c-Pentyl | H | benzo[c][1,2,5]oxadiazol-5-y1 |

### DEFINITIONS

Acyl is an alkyl-C(O)— group. Examples of acyl groups include acetyl and propionyl

Aryl is a carbocyclic aromatic ring. Examples of aryl include phenyl and napthyl

Alkyl is meant to denote a linear or branched saturated aliphatic C₁-C₇ hydrocarbon which may contain up to 3 fluorine atoms. Examples of alkyl groups include but are not limited to methyl, trifluoromethyl, ethyl, trifluoroethyl, isobutyl, neopentyl, C₁-C₄ alkyl is the subset of alkyl limited to a total of up to 4 carbon atoms.

Alkenyl is meant to denote a linear or branched aliphatic C₁-C₇ hydrocarbon which contains 1 carbon—carbon double bond. The group may also contain up to 3 fluorine atoms. Unsaturation may be internal or terminally located and both cis and trans isomers are included. Examples of which include but are limited to allyl, cis- and trans-2-butenyl, isobutenyl.

Alkynyl is meant to denote a linear or branched aliphatic C₁-C₇ hydrocarbon which contains 1 carbon—carbon tripe bond. The group may also contain up to 3 fluorine atoms. Unsaturation may be internal or terminally located. Examples of which include but are limited to propargyl and 3,3,3-trifluoroprop-1-ynyl.

The term “C₃₋₇-cycloalkyl” denotes a saturated cyclic alkyl group (saturated or partially unsaturated) having a ring size from 3 to 7 carbon atoms. Examples of said cycloalkyl include cyclopropyl, cyclobutyl, cyclopentyl, cyclohexyl, methylcyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc

Cycloalkylalkyl is a cycloalkyl group attached to a C1-C4 alkyl spacer group. Examples include cyclopropylmethyl, cyclobutylmethyl, cyclopropylethyl, cyclohexylmethyl and cyclohexylethyl.

Alkoxy is an alkyl-O— group wherein alkyl is as defined above. Examples of alkoxy groups include methoxy, trifluoromethoxy, ethoxy, trifluoroethoxy, and propoxy. For parts of the range “C₁₋₇-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₇-alkoxy, C₄₋₅-alkoxy, etc

Cycloalkoxy is a cycloalkyl-O group wherein cycloalkyl is as defined above. Examples of cycloalkoxy groups include cyclopropyloxy, cyclopentyloxy and cyclohexyloxy.

Alkylthio is alkyl-S—, cycloalkyl-S— or cycloalkylmethyl-S— wherein alkyl, cycloalkyl and alkylcycloalkyl are as defined above.

Alkylsulfonyl is alkyl-SO₂—, cycloalkyl-S O₂— or cycloalkylmethyl-S O₂— wherein alkyl-S— alkyl-S—, cycloalkyl-S— or cycloalkylmethyl-S— wherein alkyl, cycloalkyl and alkylcycloalkyl are as defined above.

Alkylamino is alkyl-NH— cycloalkyl-NH— or cycloalkylmethyl-NH— wherein alkyl, cycloalkyl and alkylcycloalkyl are as defined above.

Dialkylamino is (alkyl)₂-N—.

Oxo is an oxygen atom divalent attached to a single atom. For example a C-oxo is a carbonyl C═O and a S-oxo is S═O. Two oxo groups can attached be attached to the same S atom giving SO₂.

A “halogen” is defined as Fluoro, Chloro, Bromo or Iodo. In some instances a “halogen” is defined as Fluoro or Chloro.

A heteroatom is defined as Nitrogen Oxygen or Sulfur atom.

Heteroaryl is a mono- or bi-cyclic ring system, only one ring need be aromatic, comprising 5 to 10 ring atoms selected from C, N, O and S, provided that not more than 3 ring atoms in any single ring are other than C. Examples of heteroaryl groups include but are not limited to 1,2,3-oxadiazyl, 1,2,3-thiadiazyl, 1,2,3-triazyl, 1,2,4-oxadiazyl, 1,2,4-thiadiazyl, 1,2,4-triaziyl, 1,2,5-oxadiazyl, 1,2,5-thiadiazyl, 1,3,4-oxadiazyl, 1,3,4-thiadiazyl, 1,3,5-triazine, 1H-1,2,3-triazyl, 1H-1,2,4-triazyl, 1H-imidazyl, 1H-pyrazyl, 1H-pyrroyl, 1H-tetrazyl, furyl, isothiazyl, isoxazyl, oxazyl, pyrazyl, pyridazyl, pyridyl, pyrimidyl, thiazyl, thiophenyl, 1,5-naphthyridyl, 6-naphthyridyl, 1,7-naphthyridyl, 1,8-naphthyridyl, 2,6-naphthyridyl, 2,7-naphthyridyl, cinnolyl, isoquinolyl, phthalazyl, quinazolyl, quinolyl, quinoxalyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-d]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-d]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridyl, 2H-pyrazolo[4,3-b]pyridyl, [1,2,3]triazolo[1,5-a]pyridyl, [1,2,4]triazolo[1,5-a]pyridyl, [1,2,4]triazolo[4,3-a]pyridyl, benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridyl, 1H-imidazo[4,5-c]pyridyl, 1H-pyrazolo[3,4-b]pyridyl, 1H-pyrazolo[3,4-c]pyridyl, 1H-pyrazolo[4,3-b]pyridyl, 1H-pyrazolo[4,3-c]pyridyl, 1H-pyrrolo[2,3-b]pyridyl, 1H-pyrrolo[2,3-c]pyridyl, 1H-pyrrolo[3,2-b]pyridyl, 1H-pyrrolo[3,2-c]pyridyl, 2H-indazoyl, 3H-imidazo[4,5-b]pyridyl, 3H-imidazo[4,5-c]pyridyl, benzo[c]isothiazyl, benzo[c]isoxazyl, furo[2,3-b]pyridyl, furo[2,3-c]pyridyl, furo[3,2-b]pyridyl, furo[3,2-c]pyridiyl, isothiazolo[4,5-b]pyridyl, isothiazolo[4,5-c]pyridyl, isothiazolo[5,4-b]pyridyl, isothiazolo[5,4-c]pyridyl, isoxazolo[4,5-b]pyridyl, isoxazolo[4,5-c]pyridyl, isoxazolo[5,4-b]pyridyl, isoxazolo[5,4-c]pyridyl, oxazolo[4,5-b]pyridyl, oxazolo[4,5-c]pyridyl, oxazolo[5,4-b]pyridyl, oxazolo[5,4-c]pyridyl, thiazolo[4,5-b]pyridiyl, thiazolo[4,5-c]pyridyl, thiazolo[5,4-b]pyridyl, thiazolo[5,4-c]pyridyl, thieno[2,3-b]pyridyl, thieno[2,3-c]pyridyl, thieno[3,2-b]pyridyl and thieno[3,2-c]pyridyl. If a bicyclic heteroaryl ring is substituted, it may be substituted in any ring.

A “mono or bicyclic” ring system may be defined as a saturated or unsaturated ring system which contains 4-11 ring atoms selected from C, N, O or S of which up to 4 ring atoms may be selected independently selected from N, O, or S. The ring systems include aromatic and heteroaromatic systems. Examples of suitable monocyclic systems include but is not limited to include; phenyl, cyclopentyl, cylcohexyl, cycloheptyl, morpholinyl, piperidinyl, tetrahydroquinyl, tetrahydroisoquinoyl, pyrrolyl, furyl, thienyl, imidazyl, pyrazyl, isothiazyl, isoxazoyl, oxazolyl, thiazole, 1,2,3-triazolyl, 1,2,4-triazoyl, 1,2,3-oxadiazolyl, 1,2,4-oxadiazolyl, 1,2,5-oxadiazole, 1,3,4-oxadiazolyl, 1,2,3-thiadiazolyl, 1,2,4-thiadiazolyl, 1,2,5-thiadiazolyl, 1,3,4-thiadiazolyl, tetrazolyl, 1,2,3,4-oxatriazolyl, 1,2,3,5-oxatriazolyl, 1,2,3,4-thiatriazolyl, 1,2,3,5-thiatriazolyl tetrazolyl, 1,2,3-triazinyl compound, 1,2,4-triazinyl, 1,3,5-triazinyl, pyrazinyl, pyridazinyl or pyrimidinyl.

A “5 membered heteroaromatic ring” is defined as a an aromatic ring system containing 5 ring atoms of which up to 4 of these atoms may be heteroatoms. Examples of 5-membered heteroaromatic rings include: pyrrolyl, furyl, thienyl, imidazyl, pyrazyl, isothiazyl, isoxazoyl, oxazolyl, thiazole, 1,2,3-triazolyl, 1,2,4-triazoyl, 1,2,3-oxadiazolyl, 1,2,4-oxadiazolyl, 1,2,5-oxadiazole, 1,3,4-oxadiazolyl, 1,2,3-thiadiazolyl, 1,2,4-thiadiazolyl, 1,2,5-thiadiazolyl, 1,3,4-thiadiazolyl, tetrazolyl, 1,2,3,4-oxatriazolyl, 1,2,3,5-oxatriazolyl, 1,2,3,4-thiatriazolyl, 1,2,3,5-thiatriazolyl or tetrazolyl.

A “6 membered heteroaromatic ring” is defined as an aromatic ring system containing 6 ring atoms of which up to three of these ring atoms may be heteroatoms. Examples of 6-membered heteroaromatic rings include: 1,2,3-triazinyl compound, 1,2,4-triazinyl, 1,3,5-triazinyl, pyrazinyl, pyridazinyl or pyrimidinyl.

The term “heteroaryl” refers to a mono- or bicyclic aromatic ring system, only one ring need be aromatic, and the said heteroaryl moiety can be linked to the remainder of the molecule via a carbon or nitrogen atom in any ring, and having from 5 to 10 ring atoms (mono- or bicyclic), in which one or more of the ring atoms are other than carbon, such as nitrogen, sulfur, oxygen and selenium. Examples of such heteroaryl rings include but are not limited to 1,2,3-oxadiazyl, 1,2,3-thiadiazyl, 1,2,3-triazyl, 1,2,4-oxadiazyl, 1,2,4-thiadiazyl, 1,2,4-triaziyl, 1,2,5-oxadiazyl, 1,2,5-thiadiazyl, 1,3,4-oxadiazyl, 1,3,4-thiadiazyl, 1,3,5-triazine, 1H-1,2,3-triazyl, 1H-1,2,4-triazyl, 1H-imidazyl, 1H-pyrazyl, 1H-pyrroyl, 1H-tetrazyl, furyl, isothiazyl, isoxazyl, oxazyl, pyrazyl, pyridazyl, pyridyl, pyrimidyl, thiazyl, thiophenyl, 1,5-naphthyridyl, 6-naphthyridyl, 1,7-naphthyridyl, 1,8-naphthyridyl, 2,6-naphthyridyl, 2,7-naphthyridyl, cinnolyl, isoquinolyl, phthalazyl, quinazolyl, quinolyl, quinoxalyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-d]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-d]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridyl, 2H-pyrazolo[4,3-b]pyridyl, [1,2,3]triazolo[1,5-a]pyridyl, [1,2,4]triazolo[1,5-a]pyridyl, [1,2,4]triazolo[4,3-a]pyridyl, benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridyl, 1H-imidazo[4,5-c]pyridyl, 1H-pyrazolo[3,4-b]pyridyl, 1H-pyrazolo[3,4-c]pyridyl, 1H-pyrazolo[4,3-b]pyridyl, 1H-pyrazolo[4,3-c]pyridyl, 1H-pyrrolo[2,3-b]pyridyl, 1H-pyrrolo[2,3-c]pyridyl, 1H-pyrrolo[3,2-b]pyridyl, 1H-pyrrolo[3,2-c]pyridyl, 2H-indazoyl, 3H-imidazo[4,5-b]pyridyl, 3H-imidazo[4,5-c]pyridyl, benzo[c]isothiazyl, benzo[c]isoxazyl, furo[2,3-b]pyridyl, furo[2,3-c]pyridyl, furo[3,2-b]pyridyl, furo[3,2-c]pyridiyl, isothiazolo[4,5-b]pyridyl, isothiazolo[4,5-c]pyridyl, isothiazolo[5,4-b]pyridyl, isothiazolo[5,4-c]pyridyl, isoxazolo[4,5-b]pyridyl, isoxazolo[4,5-c]pyridyl, isoxazolo[5,4-b]pyridyl, isoxazolo[5,4-c]pyridyl, oxazolo[4,5-b]pyridyl, oxazolo[4,5-c]pyridyl, oxazolo[5,4-b]pyridyl, oxazolo[5,4-c]pyridyl, thiazolo[4,5-b]pyridiyl, thiazolo[4,5-c]pyridyl, thiazolo[5,4-b]pyridyl, thiazolo[5,4-c]pyridyl, thieno[2,3-b]pyridyl, thieno[2,3-c]pyridyl, thieno[3,2-b]pyridyl, thieno[3,2-c]pyridyl, imidazo[2,1-b][1,3]thiazolyl, and 3,4-dihydro-2H-1,5-benzodioxepinyl.

If a bicyclic heteroaryl ring is substituted, it may be substituted in any ring.

The term “heterocyclic” refers to a non-aromatic (i.e., partially or fully saturated) mono- or bicyclic ring system having 4 to 10 ring atoms with at least one heteroatom such as O, N, or S, and the remaining ring atoms are carbon. Examples of heterocyclic groups include 1,2,3,4-tetrahydro-2,6-naphthyridyl, 1,2,3,4-tetrahydro-2,7-naphthyridyl, 4,5,6,7-tetrahydro-1H-imidazo[4,5-c]pyridyl, 4,5,6,7-tetrahydro-1H-pyrazolo[3,4-c]pyridyl, 4,5,6,7-tetrahydro-1H-pyrazolo[4,3-c]pyridyl, 4,5,6,7-tetrahydro-1H-pyrrolo[2,3-c]pyridyl, 4,5,6,7-tetrahydro-1H-pyrrolo[3,2-c]pyridyl, 4,5,6,7-tetrahydro-3H-imidazo[4,5-c]pyridyl, 4,5,6,7-tetrahydrofuro[2,3-c]pyridyl, 4,5,6,7-tetrahydrofuro[3,2-c]pyridyl, 4,5,6,7-tetrahydroisothiazolo[4,5-c]pyridine, 4,5,6,7-tetrahydroisothiazolo[5,4-c]pyridyl, 4,5,6,7-tetrahydroisoxazolo[4,5-c]pyridyl, 4,5,6,7-tetrahydroisoxazolo[5,4-c]pyridyl, 4,5,6,7-tetrahydrooxazolo[4,5-c]pyridyl, 4,5,6,7-tetrahydrooxazolo[5,4-c]pyridyl, 4,5,6,7-tetrahydrothiazolo[4,5-c]pyridyl, 4,5,6,7-tetrahydrothiazolo[5,4-c]pyridine, 4,5,6,7-tetrahydrothieno[2,3-c]pyridyl, 4,5,6,7-tetrahydrothieno[3,2-c]pyridyl, 5,6,7,8-tetrahydro-1,6-naphthyridyl, 5,6,7,8-tetrahydro-1,7-naphthyridyl, 5,6,7,8-tetrahydropyrido[3,4-c]pyridazyl, 5,6,7,8-tetrahydropyrido[3,4-d]pyridazine, 5,6,7,8-tetrahydropyrido[3,4-d]pyrimidyl, 5,6,7,8-tetrahydropyrido[4,3-b]pyrazyl, 5,6,7,8-tetrahydropyrido[4,3-c]pyridazyl, 5,6,7,8-tetrahydropyrido[4,3-d]pyrimidyl, 2,3,4,5-tetrahydro-1H-benzo[c]azepin-1-yl 3,4-dihydroisoquinolin-1(2H)-onyl, 3,4-dihydroquinolin-2(1H)-onyl, 3,4-dihydroquinoxalin-2(1H)-onyl, 4,5-dihydro-1H-benzo[b][1,4]diazepin-2(3H)-onyl, 4,5-dihydro-1H-benzo[b]azepin-2(3H)-onyl, indolin-2-onyl, isoindolin-1-onyl, 1,2,3,4-tetrahydroisoquinolinyl, 1,2,3,4-tetrahydroquinolinyl, 1,2,3,4-tetrahydroquinoxalinyl, 2,3,4,5-tetrahydro-1H-benzo[b][1,4]diazepinyl, 2,3,4,5-tetrahydro-1H-benzo[b]azepinyl, 2,3,4,5-tetrahydro-1H-benzo[c]azepinyl, 2,3,4,5-tetrahydrobenzo[b][1,4]oxazepinyl, 2,3,4,5-tetrahydrobenzo[b][1,4]thiazepinyl, 3,4-dihydro-2H-benzo[b][1,4]oxazinyl, 3,4-dihydro-2H-benzo[b][1,4]thiazinyl, indolinyl, isoindolinyl, 2,3-dihydrobenzo[b][1,4]oxazepin-4(5H)-only, 2,3-dihydrobenzo[b]oxepin-4(5H)-onyl, 2H-benzo[b][1,4]oxazin-3(4H)-onyl, 3,4-dihydro-2H-benzo[b][1,4]oxathiepin-2-onyl, 3,4-dihydro-2H-benzo[b][1,4]oxazin-2-onyl, 3,4-dihydrobenzo[b]oxepin-5(2H)-onyl, 4,5-dihydrobenzo[b][1,4]oxazepin-2(3H)-onyl, 4,5-dihydrobenzo[b]oxepin-2(3H)-onyl, 4,5-dihydrobenzo[b]oxepin-3(2H)-onyl, 4,5-dihydrobenzo[c]oxepin-1(3H)-onyl, benzo[b][1,4]oxathiin-2(3H)-onyl, benzofuran-2(3H)-onyl, benzofuran-3(2H)-onyl, chroman-2-onyl, chroman-3-onyl, chroman-4-onyl, isobenzofuran-1(3H)-onyl, isochroman-1-onyl, 1,3,4,5-tetrahydrobenzo[c]oxepinyl, 1,3-dihydroisobenzofuranyl, 2,3,4,5-tetrahydrobenzo[b]oxepinyl, 2,3-dihydrobenzo[b][1,4]oxathiinyl, 2,3-dihydrobenzofuranyl, 3,4-dihydro-2H-benzo[b][1,4]oxathiepinyl, chromanyl, isochromanyl, 1,4-diazepan-5-onyl, 1,4-oxazepan-2-onyl, 1,4-oxazepan-5-onyl, 1,4-thiazepan-5-onyl, azepan-2-onyl, azepan-3-onyl, azepan-4-onyl, azetidin-2-onyl, azetidin-3-onyl, morpholin-2-onyl, morpholin-3-onyl, piperazin-2-onyl, piperidin-2-onv, piperidin-3-onyl, piperidin-4-onyl, pyrrolidin-2-onyl, pyrrolidin-3-onyl, thiomorpholin-3-onyl, 3-azabicyclo[3.1.0]hexanyl, 2-azabicyclo[3.1.0]hexanyl, 1,4-diazepanyl, 1,4-oxazepanyl, 1,4-thiazepanyl, 1-azabicyclo[2.1.1]hexanyl, 1-azabicyclo[2.2.1]heptanyl, 2-azabicyclo[2.1.1]hexanyl, 2-azabicyclo[2.2.1]heptanyl, 2-azabicyclo[2.2.2]octanyl, 5-azabicyclo[2.1.1]hexanyl, 7-azabicyclo[2.2.1]heptanyl, azepanyl, azetidinvzyl, morpholinyl, piperazinyl, piperidinyl, pyrrolidinyl, quinuclidinyl, thiomorpholinyl, 1,4-dioxan-2-onyl, 1,4-dioxepan-2-onyl, 1,4-dioxepan-5-onyl, 1,4-oxathian-2-onyl, 1,4-oxathiepan-7-onyl, 1,4-oxazepan-7-onyl, morpholin-2-onyl, 3-oxabicyclo[3.1.0]hexanyl, (1S,5R)-2-oxabicyclo[3.1.0]hexanyl, 1,4-dioxanyl, 1,4-dioxepanyl, 1,4-oxathianyl, 1,4-oxathiepanyl, 2-oxabicyclo[2.1.1]hexanyl, 2-oxabicyclo[2.2.1]heptanyl, 2-oxabicyclo[2.2.2]octanyl, 5-oxabicyclo[2.1.1]hexanyl, 7-oxabicyclo[2.2.1]heptanyl, oxepanyl, oxetanyl, tetrahydro-2H-pyranyl, tetrahydrofuranyl and groups.

When present in heterocyclic groups, the sulfur atom may optionally be in an oxidized form (i.e., S═O or O═S═O).

“Heterocyclyl” is a non-aromatic mono or bicyclic ring system which is defined as a saturated or unsaturated ring system which contains 4-11 ring atoms selected from C, N, O or S of which up to 4 ring atoms may be selected independently selected from N, O, or S and at least 3 ring atoms must be C. Examples of “Heterocyclyl” ring systems include

1,4-diazepan-5-onyl, 1,4-oxazepan-2-onyl, 1,4-oxazepan-5-onyl, 1,4-thiazepan-5-onyl, azepan-2-onyl, azepan-3-onyl, azepan-4-onyl, azetidin-2-onyl, azetidin-3-onyl, morpholin-2-onyl, morpholin-3-onyl, piperazin-2-onyl, piperidin-2-onv, piperidin-3-onyl, piperidin-4-onyl, pyrrolidin-2-onyl, pyrrolidin-3-onyl, thiomorpholin-3-onyl, 3-azabicyclo[3.1.0]hexanyl, 2-azabicyclo[3.1.0]hexanyl, 1,4-diazepanyl, 1,4-oxazepanyl, 1,4-thiazepanyl, 1-azabicyclo[2.1.1]hexanyl, 1-azabicyclo[2.2.1]heptanyl, 2-azabicyclo[2.1.1]hexanyl, 2-azabicyclo[2.2.1]heptanyl, 2-azabicyclo[2.2.2]octanyl, 5-azabicyclo[2.1.1]hexanyl, 7-azabicyclo[2.2.1]heptanyl, azepanyl, azetidinvzyl, morpholinyl, piperazinyl, piperidinyl, pyrrolidinyl, quinuclidinyl, thiomorpholinyl, 1,4-dioxan-2-onyl, 1,4-dioxepan-2-onyl, 1,4-dioxepan-5-onyl, 1,4-oxathian-2-onyl, 1,4-oxathiepan-7-onyl, 1,4-oxazepan-7-onyl, morpholin-2-onyl, 3-oxabicyclo[3.1.0]hexanyl, (1S,5R)-2-oxabicyclo[3.1.0]hexanyl, 1,4-dioxanyl, 1,4-dioxepanyl, 1,4-oxathianyl, 1,4-oxathiepanyl, 2-oxabicyclo[2.1.1]hexanyl, 2-oxabicyclo[2.2.1]heptanyl, 2-oxabicyclo[2.2.2]octanyl, 5-oxabicyclo[2.1.1]hexanyl, 7-oxabicyclo[2.2.1]heptanyl, oxepanyl, oxetanyl, tetrahydro-2H-pyranyl and tetrahydrofuranyl

Heterocycloalkyl is a monocyclic saturated or partially unsaturated ring system comprising 5-6 ring atoms C, N, O and S, provided that not more than 2 ring atoms in any single ring are other than C. In the case where the heterocycloalkyl group contains a nitrogen atom the nitrogen may be substituted with an alkyl or acyl group.

Heterocycloalkyl groups may be substituted with a hydroxyl group, and alkoxy group and up to two carbonyl groups. Heterocycloalkyl groups may be linked via either carbon or nitrogen ring atoms. Examples of heterocycloalkyl groups include tetrahydrofuranyl, pyrrolidinyl, pyrrolidonyl, succinimidyl, piperidinyl, piperazinyl, N-methylpiperazinyl and morpholinyl.

Heterocycloalkylalkyl is a heterocycloalkyl group attached to a C₁-C₄ alkyl spacer.

Heterocycloakyloxy is a heterocycloalkyl-0 group.

Heteroarylalkyl is a heteroaryl group attached to a C₁-C₄ alkyl spacer.

Heteroaryloxy is a heteroaryl-0 group.

“Het²” is a heteroaryl bi-cyclic ring system, in which both rings are aromatic 8-10 ring atoms selected from C, N, O and S, provided that not more than 3 ring atoms in any single ring are other than C. Examples of heteroaryl groups include but are not limited to 1,5-naphthyridyl, 6-naphthyridyl, 1,7-naphthyridyl, 1,8-naphthyridyl, 2,6-naphthyridyl, 2,7-naphthyridyl, cinnolyl, isoquinolyl, phthalazyl, quinazolyl, quinolyl, quinoxalyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-d]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-d]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridyl, 2H-pyrazolo[4,3-b]pyridyl, [1,2,3]triazolo[1,5-a]pyridyl, [1,2,4]triazolo[1,5-a]pyridyl, [1,2,4]triazolo[4,3-a]pyridyl, benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridyl, 1H-imidazo[4,5-c]pyridyl, 1H-pyrazolo[3,4-b]pyridyl, 1H-pyrazolo[3,4-c]pyridyl, 1H-pyrazolo[4,3-b]pyridyl, 1H-pyrazolo[4,3-c]pyridyl, 1H-pyrrolo[2,3-b]pyridyl, 1H-pyrrolo[2,3-c]pyridyl, 1H-pyrrolo[3,2-b]pyridyl, 1H-pyrrolo[3,2-c]pyridyl, 2H-indazoyl, 3H-imidazo[4,5-b]pyridyl, 3H-imidazo[4,5-c]pyridyl, benzo[c]isothiazyl, benzo[c]isoxazyl, furo[2,3-b]pyridyl, furo[2,3-c]pyridyl, furo[3,2-b]pyridyl, furo[3,2-c]pyridiyl, isothiazolo[4,5-b]pyridyl, isothiazolo[4,5-c]pyridyl, isothiazolo[5,4-b]pyridyl, isothiazolo[5,4-c]pyridyl, isoxazolo[4,5-b]pyridyl, isoxazolo[4,5-c]pyridyl, isoxazolo[5,4-b]pyridyl, isoxazolo[5,4-c]pyridyl, oxazolo[4,5-b]pyridyl, oxazolo[4,5-c]pyridyl, oxazolo[5,4-b]pyridyl, oxazolo[5,4-c]pyridyl, thiazolo[4,5-b]pyridiyl, thiazolo[4,5-c]pyridyl, thiazolo[5,4-b]pyridyl, thiazolo[5,4-c]pyridyl, thieno[2,3-b]pyridyl, thieno[2,3-c]pyridyl, thieno[3,2-b]pyridyl and thieno[3,2-c]pyridyl. If a bicyclic heteroaryl ring is substituted, it may be substituted in any ring.

In the case compounds of Formula (I-XI) may contain asymmetric centers and exist as different enantiomers or diastereomers. All enantiomers or diastereomeric forms are embodied herein.

Compounds in the disclosure may be in the form of pharmaceutically acceptable salts. The phrase “pharmaceutically acceptable” refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary, secondary and tertiary amines, and amino acids. Salts derived from inorganic acids include sulfuric, hydrochloric, phosphoric, methanesulphonic, hydrobromic. Salts derived from organic acids include C₁₋₆ alkyl carboxylic acids, di-carboxylic acids and tricarboxylic acids such as acetic acid, propionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid.

Compounds in the disclosure may be in the form of a solvates. This occurs when a compound of formula (I-IX)) crystallizes in a manner that it incorporates solvent molecules into the crystal lattice. Examples of solvents forming solvates are water (hydrates), MeOH, EtOH, iPrOH, and acetone.

Compounds in the disclosure may exist in different crystal forms known as polymorphs

Practitioners of the art will recognize that certain chemical groups may exist in multiple tautomeric forms. The scope of this disclosure is meant to include all such tautomeric forms. For example, a tetrazole may exist in two tautomeric forms, 1-H tetrazole and a 2-H tetrazole. This is depicted in FIGURE below. This example is not meant to be limiting in the scope of tautomeric forms.

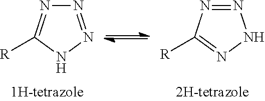

Practitioners of the art will recognize that certain electrophilic ketones, may exist in a hydrated form. The scope of this disclosure is to include all such hydrated forms. For example, a trifluoromethyl ketone may exist in a hydrated form via addition of water to the carbonyl group.

### General Experimental Schemes

### Abbreviations

Abbreviations used in the following examples and preparations include:
Aβ Amyloid-beta
ABL Aβ lowering
Ac acyl (Me-C(O)—)
AD Alzheimer's Disease
APP Amyloid Precursor Protein
Bn Benzyl
b/p brain/plasma
BSA Bovine serum Albumin
c Cyclo
calcd. Calculated
cBu Cylcobutyl
c-Bu Cylcobutyl
cₘₐₓ Maximal concentration
cPr Cyclopropyl
c-Pr Cyclopropyl
CHAPS 3-[3-cholamidopropyl)-dimethyl-ammonio]-1-propane sulfonate
CTF Carboxy Terminal Fragment
CSF Cerebrospinal fluid
DCC N′N′Dicyclohexylcarbodiimide
DCM Dichloromethane (methylene chloride)
DEA Di-ethylamine
DIEA Di-isopropylethyl amine
DMAP 4-Dimethylamino Pyridine
DMF Dimethylformamide
DMSO Dimethyl sulfoxide
Dppf 1,4-Bis(diphenylphosphino) ferrocene
EDC 1-(3-Dimethylaminopropyl)-3-ethylcarbodiimide Hydrochloride
EDTA Ethylene Diamine Tetra-acetic Acid
ELISA Enzyme-Linked Immuno Sorbent Assay
Et₃N Triethylamine
Eq. Equivalent
g gram(s)
HOBt 1-Hydroxybenzotriazole
HPLC High Pressure Liquid Chromatography
h Hour(s)
hr Hour(s)
i.v or IV. Intravenous
KHMDS Potassium Hexamethydisilazide
LC-MS Liquid Chromatography-Mass Spectrometry
LDA Lithium Di-isopropylamide
m Multiplet
MeOH Methyl Alcohol or Methanol
m meta
mcpba meta-chloro perbenzoic acid
min Minute(s)
mmol millimoles
mmole millimoles
ul Microliter
μl microliter
Ms Mesylate
MS Mass Spectrometry
MW Molecular Weight (all values are ±0.05)
n normal
NBS N-Bromosuccinamide
NIS N-Iodosuccinamide
NMR Nuclear Magnetic Resonance
NMM N-Methyl Morpholine
NSAIDS Non-Steroidal Anti-Inflammatory Drugs
ortho
o/n overnight
p para
PBS Phosphate Buffered Saline
PEPPSI 1,3-Bis(2,6-diisopropylphenyl)imidazolidene)(3-chloropyridyl) palladium(II) dichloride
PhNTf₂ 1,1,1-trifluoro-N-phenyl-N-(trifluoromethylsulfonyl)methanesulfonamide
POPd Dihydrogen dichlorobis(di-tert-butylphosphinito-kp) palladate (2-)
p.s.i. Pounds per square inch
PPAA 1-Propanephosphonic Acid Cyclic Anhydride
PyBOP® Benzotriazol-1-yl-oxytripyrrolidinophosphonium hexafluorophosphate
PK Pharmacokinetics
RT (or rt) room temperature (about 20-25° C.)
s Singlet
sat. Saturated
sec secondary
t Triplet
tert tertiary
TBAF Tetra-butyl ammonium fluoride
TFA Trifluoroacetic Acid
THF Tetrahydrofuran
TMB 3,3′ 5, 5′ Tetramethylbenzidine
TMS Trimethylsilyl
Tf Triflate
Ts Tosylate
v/v volume/volume
wt/v weight/volume

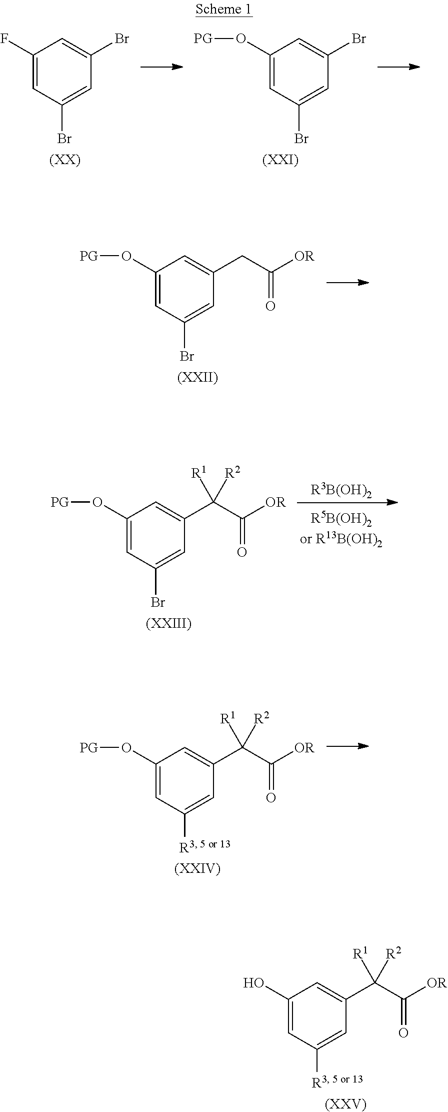

1,3-dibromo-5-fluorobenzene (XX) is treated with a protected “OH source” such as benzyl alcohol or MeOH in the presence of a base such as K2CO3, Cs₂CO₃, LiHMDs, NaH, LDA or KHMDs. The reaction is run an inert solvent such as THF, dioxane or DMF at a temperature of 0-120° C. The dibromoaromatic (XXI) is transformed into the phenylacetic derivative (XXII) by treatment with diethyl malonate in the presence of a base such as K₂CO₃, Cs₂CO₃, LiHMDs, NaH, LDA or KHMDs and a copper (I) salt, such as CuBr. The reaction is run in an inert solvent such as THF, dioxane, DMSO or DMF at a temperature of 0-120° C., a catalyst such as proline may be added to the reaction. The reaction mixture is subjected to AcOH at a temperature of 30-120° C. to effect de-carboxylation to give the compounds of formula (XXII), where R is H, C₁₋₆ alkyl, benzyl or substituted benzyl. Practitioners of the art will recognize that if only one of R¹ and R²═H, then compound (XXI) may be taken directly to compound (XXIV) by the appropriate choice of a substituted malonate derivate. The phenyl acetic esters of formula (XXII) are alkylated by treatment with a base such as NaOH, LiHMDs, NaH, ^{t}BuOK, LDA or KHMDs in an inert solvent such as THF or DMF at a temperature of −78 to 20° C. followed by the addition of the appropriate alkylating agent(s), such as an alkyl halide. If in the compound of formula (XOH) both R¹ and R² are not hydrogen, a person of ordinary skill in the art will recognize that it may necessary to conduct two separate alkylation reactions in a sequential manner. If R¹ and R² are taken together to form a ring then a di-alkylating agent of such as 1,2 di-bromoethane, 1,3 di-bromopropane or 1,4 di-bromobutane may be used. The biphenyl derivative of formula (XXIV) is synthesized by treating the aromatic compounds of formula (IX) with the appropriate boronic acid in the presence of a palladium catalyst such as Pd(PPh₃)₄, PdCl₂(dppf), POPd or PEPPSI and a base such as Cs₂CO₃, KOH, CsF, NaOH or K₂CO₃. The reaction is usually carried out in a solvent such as DME, THF, toluene, water or a mixture of said solvents at a temperature of 0-120° C. The protecting group of compound (XXIV) is removed by methods known to those of ordinary skill in the art to furnish the phenol (XXV).

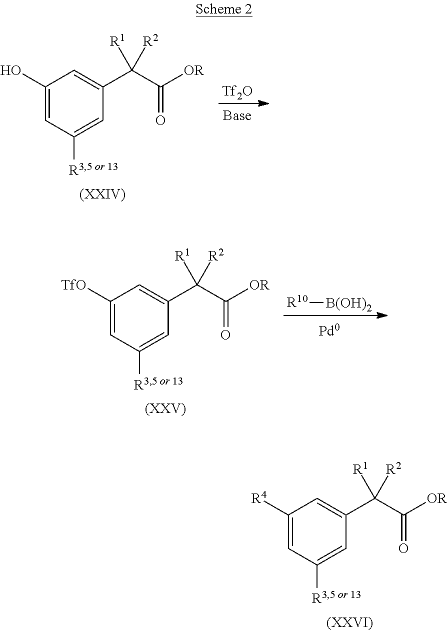

The resulting phenol (XXIV) is transformed into a triflate group by treatment with a triflating reagent such as triflic anhydride (Tf₂O) or PhNTf₂, in an inert solvent such as THF or CH₂Cl₂ in the presence of a base such as pyridine or lutidine. The reaction is usually run at a temperature of −20 to 40° C. The resultant triflate (XXV) is transformed into the compound of formula (XXVI) by treatment with the appropriate boronic acid in the presence of a palladium catalyst such as Pd(PPh₃)₄, PdCl₂(dppf), POPd or PEPPSI, a base such as Cs₂CO₃, KOH, CsF, NaOH or K₂CO₃ and a chloride source such as lithium chloride. The reaction is usually carried out in a solvent such as DME, THF, toluene, water or a mixture of said solvents at a temperature of 0-120° C.

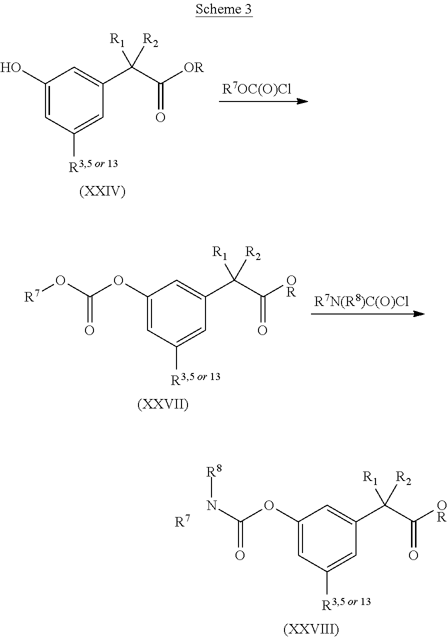

Carbonates of formula (XXVII) are prepared by treating the phenol of formula (XXIV) with a chloroformate in the presence of a base such as NaH, KHMDs, NaHMDS, LiHMDS, Et₃N or Hunigs base. The reaction is run in a solvent such as acetone, DMF, THF, dioxane or a mixture thereof. The carbamates of formula (XXVIII) are prepared by treating the phenol of formula (XXIV) with a carbonyl chloride in the presence of a base such as NaH, KHMDs, NaHMDS, LiHMDS, Et₃N or Hunigs base. In the instance where R⁸═H, the carbonyl chloride can be replaced with the appropriate isocyanate.

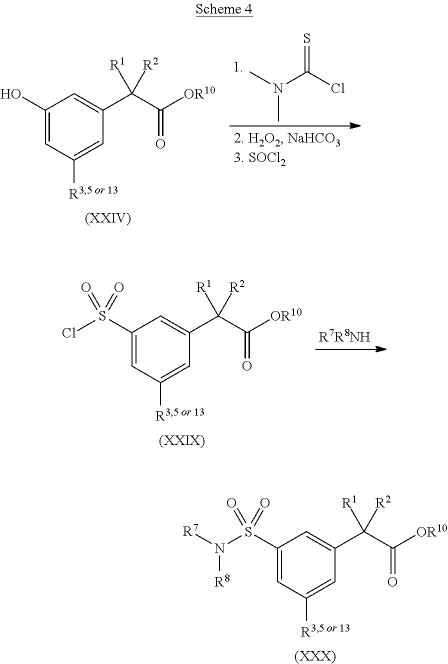

The sulfonyl chlorides of formula (XXIX) can be prepared from the phenol of formula (XXIV) by (i) treatment with dimethylcarbamothioic chloride, the reaction is usually carried out in a high boiling solvent such as xylenes, DMF, diphenyl ether, decalin, dichlorobenzene at a temperature of 50-200° C. (ii) The product is then subjected to oxidative conditions is the presence of base, such as a mixture of hydrogen peroxide and sodium bicarbonate, upon which the intermediate is converted to the sulfonyl chloride by treatment with a reagent such as thionyl chloride. The sulfonyl chlorides of formula (XXIX) are converted to the sulfonamides of formula (XXX) by treatment with an appropriate primary or secondary amine (or ammonia) in the presence of a base such as K₂CO₃, NaHCO₃, Et₃N or pyridine. The reaction is run in a solvent such as CH₂Cl₂, CHCl₃, acetone, THF, DMF, dioxane or acetonitrile at a temperature of 0-100° C. If necessary a catalyst such as DMAP may be added to the reaction

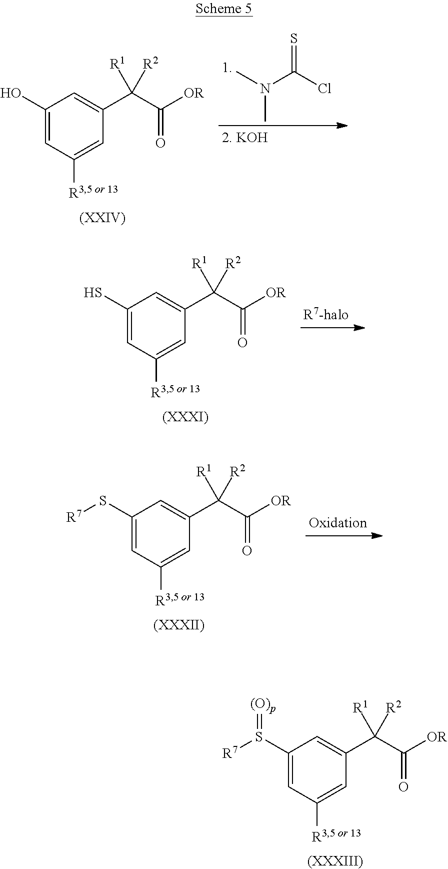

The thiol of formula (XXXI) can be prepared from the phenol of formula (XXIV) by initial treatment with dimethylcarbamothioic chloride, the reaction is usually carried out in a high boiling solvent such as xylenes, DMF, diphenyl ether, decalin, dichlorobenzene at a temperature of 50-200° C. The product is then subjected to hydrolyzing conditions usually in the presence of a base such as NaOH or KOH in a solvent system such as water, MeCN, THF, dioxane, DMF or a mixture thereof. The reaction is run at a temperature of 0-100° C. The thiol is alkylated with an appropriate electrophile to give the sulfide of formula (XXXII). The reaction is performed in the presence of a base such as NaH, KHMDs, BuLi, Et₃N or Hunigs base in a solvent such as CH₂Cl₂, MeCN, THF, DMF or DMSO at a temperature of 0-100° C. The sulfide is converted into the sulfoxides and sulfones of formula (XXXIII) by treatment with an oxidative agent such as H₂O₂ or mcpba. The reaction can be stopped at the sulfoxide stage by choice of conditions known to those of ordinary skill in the art.

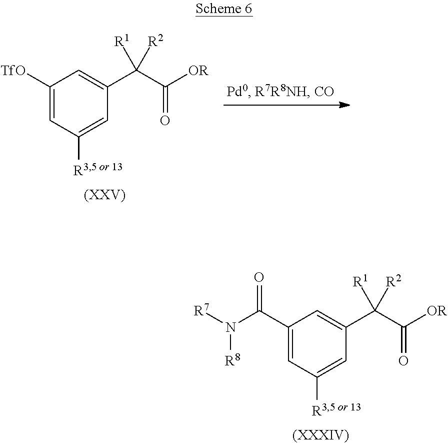

The amides of formula (XXXIV) can be prepared from the triflate (XXV) by treatment with the appropriate amine, carbon monoxide in the presence of a suitable Pd catalyst such as Pd(PPh₃)₄, PdCl₂(dppf), POPd or PEPPSI. The reaction can be run at a pressure of 1-10 atoms and at a temperature of RT-100° C. in an appropriate solvent.

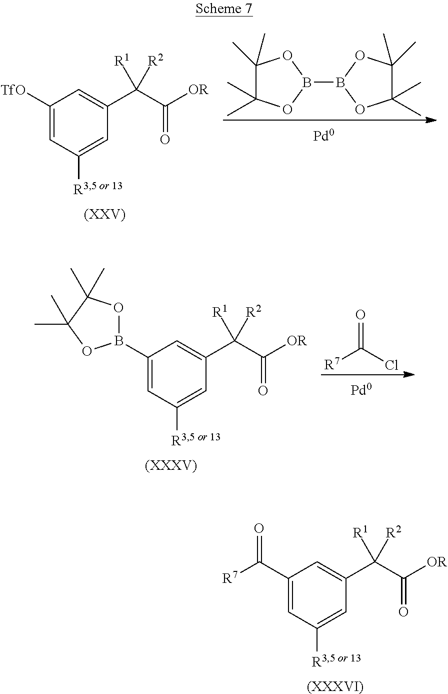

The boronate of formula (XXXV) are prepared by treatment of the triflate (XXV) with 4,4,4′,4′,5,5,5′-heptamethyl-2,2′-bi(1,3,2-dioxaborolane) in the presence of a Pd catalyst such as Pd(PPh₃)₄, PdCl₂(dppf), POPd or PEPPSI and a base. LiCl may also be added to the reaction mixture. The boronate is converted into the ketone of formula (XXXVI) by reaction with an appropriate acid chloride in the presence of a Pd catalyst such as Pd(PPh₃)₄, PdCl₂(dppf), POPd or PEPPSI. A base such as Cs₂CO₃, KOH, CsF, NaOH or K₂CO₃ is added and the reaction is performed in a solvent such as acetone, THF, toluene, dioxane, DMF, MeCN or a mixture thereof at a temperature of 0-120° C.

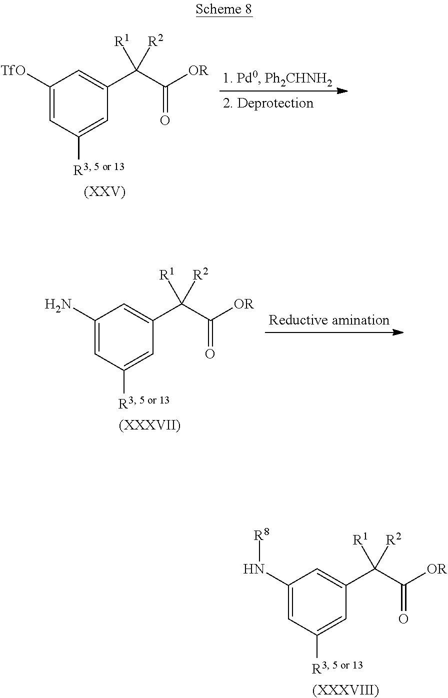

The anilines of formula (XXXVII) are prepared by treatment of the triflate (XXV) with an ammonia source such as diphenylethanamine in the presence of a suitable Pd catalyst. The free aniline is then revealed via a deprotection reaction which is well known to those of ordinary skill in the art. The aniline can undergo a reductive amination reaction with an appropriate aldehyde or ketone. The reaction is performed by in a solvent such as MeOH, CH₂Cl₂, toluene, THF, DMF, MeCN or a mixture thereof, with a reducing agent such as NaCNBH₃ or Na(OAc)₃BH. Molecular sieves or Ti(OⁱPr)₄ may be added to the reaction.

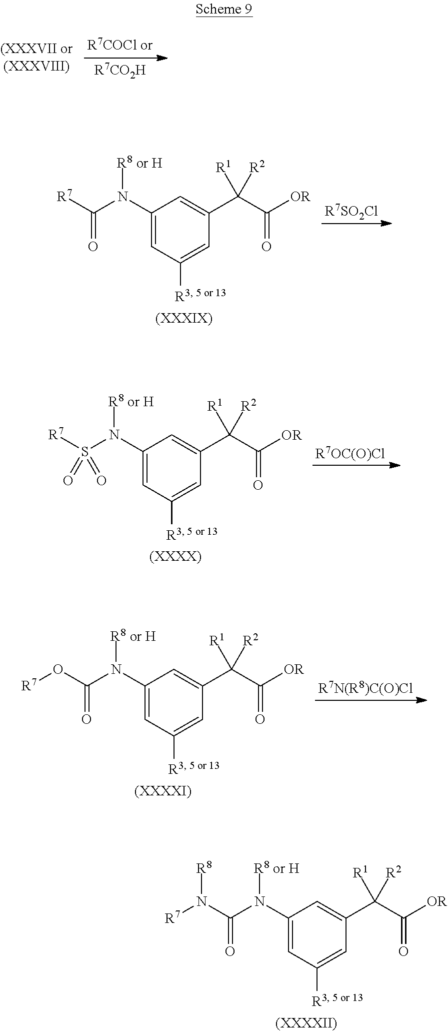

The amides (XXXIX) are synthesized by treating the anilines of formulas (XXXVII) or (XXXVIII) with an appropriate acid chloride in the presence of a base such as pyridine, Et3N, Hunigs base, NaHCO₃, K₂CO₃ in a solvent such as acetone, THF, dioxane, MeCN, CH₂Cl₂, CHCl₃, toluene, water or a mixture thereof. The reaction is usually run at a temperature of 0-100° C. Alternatively, the anilines can be treated with the appropriate carboxylic acid in the presence of a coupling agent (e.g., PyBOP, PyBrOP, dicyclohexylcarbodiimide (DCC), 1-(3′-dimethylaminopropyl)-3-ethylcarbodiimide (EDC), tosyl chloride, or 1-propanephosphonic acid cyclic anhydride (PPAA)) and a suitable base if required (e.g., triethylamine, DMAP, or N-methylmorpholine (NMM)). The reaction is performed in a solvent such as dichloromethane, chloroform, or dimethylformamide. The reaction is run at a temperature of −20 to 100° C., preferably at room temperature. Optionally, agents such as HOBt, hydroxy succinimide or SiO₂ maybe added to the reaction.

The sulfonamides of formula (XXXX) are prepared by treating the anilines of formulas (XXXVII) or (XXXVIII) with the appropriate sulfonyl chlorides. The reaction is run in the presence of a base such as K₂CO₃, NaHCO₃, Et₃N or pyridine and in a solvent such as CH₂Cl₂, CHCl₃, acetone, THF, DMF, dioxane or acetonitrile at a temperature of 0-100° C. If necessary a catalyst such as DMAP may be added to the reaction.

The carbamates of formula (XXXXI) are prepared by treating the anilines of formulas (XXXVII) or (XXXVIII) with a chloroformate in the presence of a base such as NaH, KHMDs, NaHMDS, LiHMDS, Et₃N or Hunigs base. The reaction is run in a solvent such as acetone, DMF, THF, dioxane or a mixture thereof.

The ureas of formula (XXXII) are prepared by treating the anilines of formulas (XXXVII) or (XXXVIII) with a carbonyl chloride in the presence of a base such as NaH, KHMDs, NaHMDS, LiHMDS, Et₃N or Hunigs base. In the instance where R⁸═H, the carbonyl chloride can be replaced with the appropriate isocyanate.

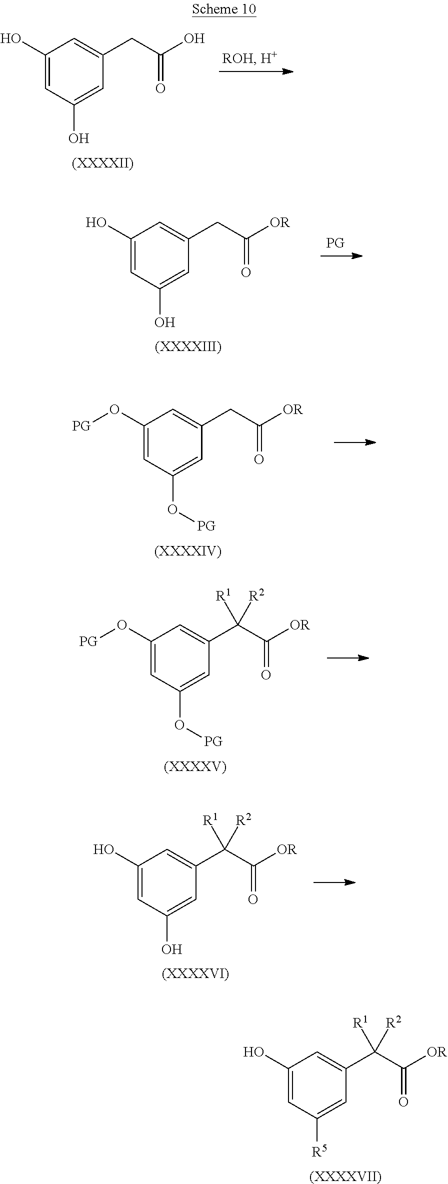

The acid of formula (XXXXII) may be protected as an ester by methods known to those of ordinary skill in the art. The resulting ester's (XXXXVII) phenols may also be protected by methods known to those of ordinary skill in the art. The ester of formula (XXXXIV) is alkylated by treatment with a base such as LiHMDs, NaH, ^{t}BuOK, LDA or KHMDs in an inert solvent such as THF or DMF at a temperature of −78 to 20° C. followed by the addition of the appropriate alkylating agent(s), such as an alkyl halide. If in the compound of formula (XXXXV) both R¹ and R² are not hydrogen, a person of ordinary skill in the art will recognize that it may necessary to conduct two separate alkylation reactions in a sequential manner. If R¹ and R² are taken together to form a ring then a di-alkylating agent of such as 1,2 di-bromoethane, 1,3 di-bromopropane or 1,4 di-bromobutane may be used. The alkylated esters of formula (XXXXV) are deprotected to reveal the phenol hydroxy groups by methods known to those of ordinary skill in the art to give the phenols of formula (XXXXVI). The phenols may be alkylated with the appropriate electrophile to give the ethers of formula (XXXXVII). The alkylation is performed in a solvent such as DMSO, DMF, acetone, THF, MeCN, toluene or a mixture thereof in the presence of a base such as BuLi, KOH, KHMDs, NaHMDs, LiHMDs, NaH K₂CO₃, Cs₂CO₃ or KO^{t}Bu. The reaction is usually run at a temperature of 0-100° C.

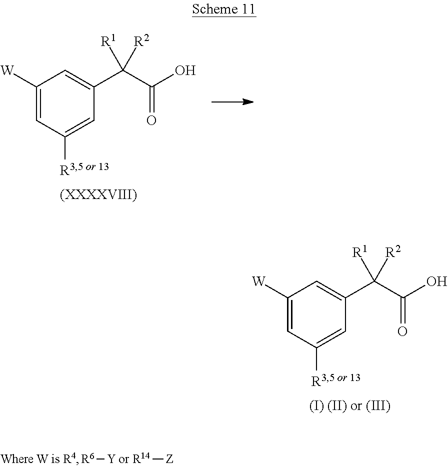

The compounds of formulas (I), (II) or (III) may be obtained via deprotection of the esters of formula (XXXXVIII) by methods known to those of ordinary skill in the art. Practitioners of the art will also recognize that the order of certain steps in the above schemes may be altered or interchanged between different reaction schemes.

Reactive groups not involved in the above processes can be protected with standard protecting groups during the reactions and removed by standard procedures (T. W. Greene & P. G. M. Wuts, Protecting Groups in Organic Synthesis, Third Edition, Wiley-Interscience) known to those of ordinary skill in the art. Presently preferred protecting groups include methyl, benzyl, acetate and tetrahydropyranyl for the hydroxyl moiety, and BOC, CBz, trifluoroacetamide and benzyl for the amino moiety, methyl, ethyl, tert-butyl and benzyl esters for the carboxylic acid moiety.

### Enantioselective Methods

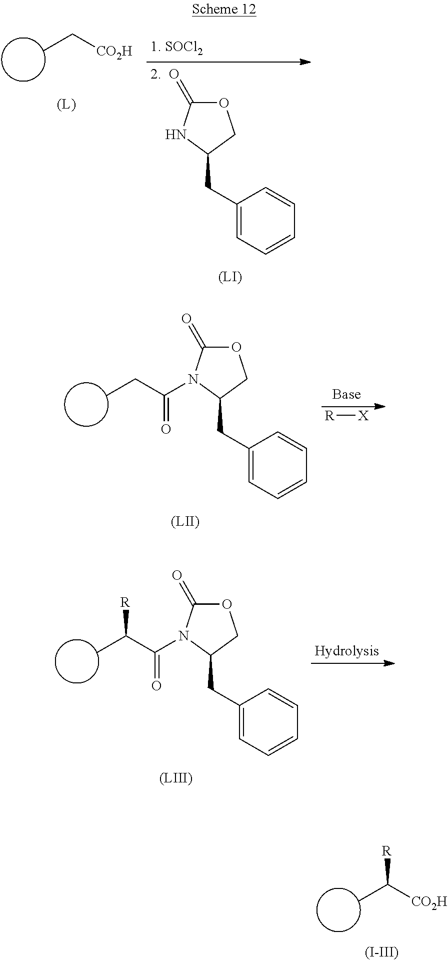

Compounds of formulas I-III may be prepared in an enantioselectively, this can be accomplished via resolution via chiral HPLC or via asymmetric synthesis. The phenyl acetic acids of formula (L) are converted into the corresponding acid chlorides, via treatment with SOCl₂ or oxalyl chloride with a catalytic amount of DMF. The reaction is performed in an inert solvent such as CH₂Cl₂, CHCl₃, THF, or toluene at a temperature of 0-80° C. The acid chloride is treated with either (R)— or (S)-4-benzyloxazolidin-2-one to (R isomer depicted-LI) give the oxazolidinone (LII). The oxazolidinone (LII) is then subjected to a base such as NaHMDs, LiHMDS, KHMDS, BuLi or KO^{t}Bu in an inert solvent such as THF, Me-THF or Et₂O at a temperature of -78 to 0° C. The subsequent enolate is then treated with the appropriate electrophile to give the alkylated oxazolidinone (LIII). The chiral auxiliary is removed under conditions such as LiOH/H₂O₂ followed by a reductive work up with a reagent such as sodium bi-sulfite to give the desired products of formulas (I-III).

### Methyl 2-(3-(benzyloxy)-5-hydroxyphenyl)acetate

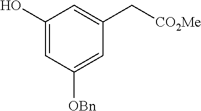

To a suspension of NaH (2.76 g, 0.057 mol) in DMF (100 ml) was slowly added a mixture of methyl 2-(3,5-dihydroxyphenyl)acetate (10 g, 0.054 mol) and benzyl chloride (7.26 g, 0.057 mol) in 50 ml of DMF at 0° C. over a period of 15 min under an atmosphere of nitrogen. Upon completion of the addition, the reaction mixture was stirred for another 30 min at 0° C., upon which it was poured onto crushed ice and extracted with EtOAc (×2). The combined organic layers were washed with water followed by brine, dried over Na₂SO₄ and concentrated in vacuo. The residue was purified by Flash column Chromatography to give methyl 2-(3-(benzyloxy)-5-hydroxyphenyl)acetate in 55% yield. (8.2 g).

or

To a stirred solution of methyl-2-(3,5-dihydroxyphenyl)acetate (30 g, 164 mmol) in 300 ml of CH₃CN, was added slowly K₂CO₃ (25 g, 183 mmol) at room temperature. The reaction mixture was cooled to 0° C. and benzyl bromide (19.5 mL, 164 mmol) was added slowly over a period of 15 min under a nitrogen atmosphere. Upon completion of the addition, the reaction mixture was allowed to warm to room temperature and stirred for a further 8 h. The reaction mixture was filtered through small bed of Celite™ pad concentrated under reduced pressure. The residue was purified by Flash column Chromatography to give methyl 2-(3-(benzyloxy)-5-hydroxyphenyl)acetate (15 g) in 35% yield along with dibenzyl compound (18 g). ¹HNMR (CDCl3, 400 MHz): 7.35-7.42 (m, 5H); 6.51 (s, 1H); 6.39 (s, 2H), 5.16 (m, 1H), 4.99 (s, 1H), 3.72 (s, 3H); 3.52 (s, 2H).

### Methyl 2-(3-(benzyloxy)-5-(trifluoromethylsulfonyloxy)phenyl)acetate

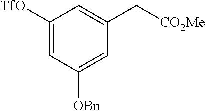

To a stirred solution of 2-(3-(benzyloxy)-5-hydroxyphenyl)acetate (700 mg, 2.57 mmol) in 50 ml of DCM was slowly added DIPEA (057 ml, 3.34 mmol) at 0° C. followed by Triflic anhydride (870 mg, 3.08 mmol). The reaction mixture was stirred for 30 min at 0° C. Upon completion of the reaction, the was mixture poured onto crushed ice and extracted with EtOAc (×2). The combined organic layers were washed with 10% NaHCO₃ solution and with water. The organic layer was dried over Na₂SO₄, filtered and evaporated to give methyl 2-(3-(benzyloxy)-5-(trifluoromethylsulfonyloxy)phenyl)acetate in 80% yield. (831.7 mg) which was used without further purification in the next step. ¹HNMR (CDCl₃): 7.42 (bs, 5H); 6.94 (s, 1H); 6.82 (bs, 2H); 5.07 (s, 2H); 3.69 (s, 3H); 3.62 (s, 2H).

### Methyl 2-(5-(benzyloxy)-4′-(trifluoromethyl)biphenyl-3-yl)acetate

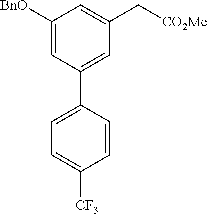

To a stirred solution of 2-(3-(benzyloxy)-5-hydroxyphenyl)acetate (2 g, 7.3 mmol) in dry DCM (50 mL) was slowly added DIPEA (1.15 mL, 9.5 mmol) at 0° C. followed by triflic anhydride (1.44 mL, 1.2 eq). The reaction mixture was stirred for 30 min at 0° C. Upon completion of the reaction, the mixture was poured onto crush ice and extracted with methylene dichloride (2×50 mL). The combined organic layers were washed with 10% NaHCO₃ solution and water. The organic layer was dried over Na₂SO₄, filtered and concentrated in vacuo methyl 2-(3-(benzyloxy)-5-(trifluoromethylsulfonyloxy)phenyl)acetate (3.5 g) which was used directly in the next step.

A mixture of methyl 2-(3-(benzyloxy)-5-(trifluoromethylsulfonyloxy)phenyl)acetate (3.5 g, 8.6 mmol), 4-Trifluoromethyl phenyl boronic acid (2.46 g, 12.9 mmol), trans dichloro bis(triphenyl phosphine) palladium (II) (1.00 g, 0.86 mmol), cesium carbonate (11.29 g, 34.6 mmol) in 1,4-dioxane:H2O (90 ml:20 mL) was stirred for 4 h at 100° C. Upon completion of reaction, the precipitate was removed by filtration. The filtrate was diluted with water and extracted with EtOAc (2×100 mL). The combined organic layers were washed with water followed by brine, dried over Na₂SO₄ and concentrated under reduced pressure. The residue was purified by Flash Column Chromatography (1:4 EtOAc:Hexane as eluent) to give methyl 2-(5-(benzyloxy)-4′-(trifluoromethyl) biphenyl-3-yl)acetate in (2.3 g). ¹HNMR (CDCl₃, 200 MHz): 7.68 (m, 2H); 7.44 (m, 2H); 7.35 (s, 1H), 5.15 (s, 2H), 3.75 (s, 3H), 3.64 (s, 2H).

### Methyl 2-(5-(benzyloxy)-4′-(trifluoromethyl)biphenyl-3-yl)-4-methyl pentanoate

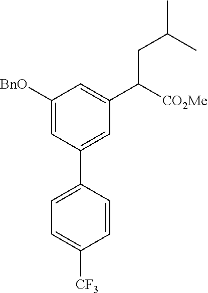

To a suspension of NaH (47 mg, 50% suspension, 0.979 mmol) in DMF at 0° C. was slowly added a mixture of methyl 2-(5-(benzyloxy)-4′-(trifluoromethyl) biphenyl-3-yl)acetate (375 mg, 0.937 mmol) and isobutyl bromide (141 mg, 1.029 mmol) as a solution in DMF (10 mL) under nitrogen atmosphere over a period of 15 min. Upon completion of the addition, the mixture was stirred for 15 min at 0° C. upon which it was poured onto crushed ice and extracted with EtOAc (×2). The combined organic layers were washed with water, dried over Na₂SO₄ and evaporated to give compound methyl 2-(5-(benzyloxy)-4′-(trifluoromethyl)biphenyl-3-yl)-4-methyl pentanoate in 75% yield (320 mg), and was used without further purification. ¹HNMR (CDCl₃): 7.68 (s, 4H); 7.42 (s, 5H); 7.15 (s, 1H); 7.14 (s, 1H); 7.08 (s, 1H); 5.13 (s, 2H); 3.72 (t, 1H); 3.69 (s, 3H); 2.02 (m, 1H); 1.71 (m, 1H); 1.48 (m, 1H); 0.93 (d, 6H).

### Methyl-2-(5-hydroxy-4′-(trifluoromethyl)biphenyl-3-yl)-4-methyl pentanoate

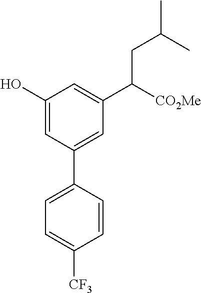

Pd/C (100 mg) was slowly added to a stirred solution of 2-(5-(benzyloxy)-4′-(trifluoromethyl)biphenyl-3-yl)-4-methyl pentanoate (1 g, 2.19 mmol) in 100 ml of MeOH under nitrogen atmosphere. The mixture was hydrogenated for 2 h, upon which the mixture was filtered through a pad of Celite™ washing with MeOH. The volatiles were removed in vacuo to give methyl-2-(5-hydroxy-4′-(trifluoromethyl)biphenyl-3-yl)-4-methyl pentanoate in 88% yield (706 mg). ¹HNMR(CDCl3): 7.66 (s, 4H); 7.12 (s, 1H); 6.97 (s, 1H); 6.87 (s, 1H); 4.98 (bs, 1H0; 3.68 (t, 1H); 3.67 (s, 3H); 2.02 (m, 1H); 1.98 (m, 1H); 1.70 (m, 1H); 0.94 (m, 1H); 0.92 (d, 6H).

### Example 47

### 4-Methyl-2-(5-(2,2,2-trifluoroethoxy)-4′-(trifluoromethyl)biphenyl-3-yl)pentanoic acid

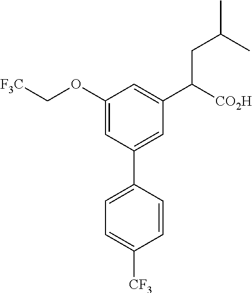

To a stirred mixture of methyl-2-(5-hydroxy-4′-(trifluoromethyl)biphenyl-3-yl)-4-methyl pentanoate (800 mg, 0.218 mmol) and K₂CO₃ (1.5 g, 10.92 mmol) of DMF (50 ml) was slowly added trifluoroethyl iodide (2.29 g, 10.92 mmol) at 0° C. over a period of 10 min. The mixture was stirred for a further 30 min at 0° C. and then heated at 100° C. for 4 h. Upon completion of the reaction, the mixture was poured into water and extracted with EtOAc (×2). The combined organic layers were washed with water, dried over Na₂SO₄ and concentrated in vacuo. The residue was purified by Flash Column Chromatography to give methyl 4-methyl-2-(5-(2,2,2-trifluoroethoxy)-4′-(trifluoromethyl)biphenyl-3-yl)pentanoate in 55% yield. (538 mg). To a solution of the product (500 mg, 1.11 mmol) in a MeOH/THF/Water mixture (10 ml/10 ml/10 ml) was added lithium hydroxide monohydrate (14 mg, 3.34 mmol). The mixture was stirred at RT for 2 h. Upon completion of the reaction, the volatiles were removed under reduced pressure, the residue was diluted with water, acidified with 5% HCl solution and extracted with EtOAc (×2). The combined organic layers were washed with water, dried with Na₂SO₄, filtered and concentrated in vacuo. The residue was purified by Flash Column Chromatography to give 4-methyl-2-(5-(2,2,2-trifluoroethoxy)-4′-(trifluoromethyl)biphenyl-3-yl)pentanoic acid in 63% yield. (305 mg). ¹HNMR (CDCl₃): 7.67 (s, 4H); 7.23 (s, 1H); 7.14 (s, 1H); 6.97 (s, 1H); 4.42 (q, 2H); 3.75 (t, 1H); 2.03 (m, 1H); 1.72 (m, 1H); 1.56 (m, 1H); 0.96 (d, 6H).

### Example 41

### 1-(5-(2,2,2-trifluoroethoxy)-4′-(trifluoromethyl)biphenyl-3-yl)cyclo butane carboxylic acid

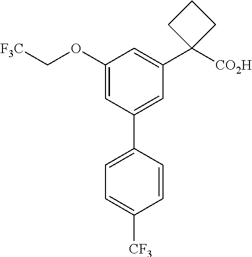

### Step 1

### Methyl 1-(5-(benzyloxy)-4′-(trifluoromethyl)biphenyl-3-yl)cyclo-butanecarboxylate

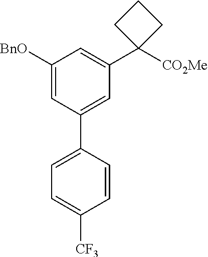

To a suspension of NaH (47 mg, 50% suspension, 0.979 mmol) in 25 ml of DMF was slowly added a mixture of methyl 2-(5-(benzyloxy)-4′-(trifluoromethyl)biphenyl-3-yl)-4-methyl pentanoate (375 mg, 0.937 mmol) and 1,3-Dibromopropane (199 mg, 0.984 mmol) in 10 ml of DMF at 0° C. under a nitrogen atmosphere for 15 min. Upon completion of the addition, the mixture was stirred for 25 min at 0° C. The mixture was poured onto crushed ice and extracted with EtOAc (×2). The combined organic layers were washed with water, dried over Na₂SO₄ and concentrated in vacuo. The residue was purified by Flash Column Chromatography to give compound methyl 1-(5-(benzyloxy)-4′-(trifluoromethyl)biphenyl-3-yl)cyclo-butanecarboxylate in 62% yield. (255 mg). ¹HNMR (CDCl3): 7.68 (s, 4H); 7.48 to 7.38 (m, 5H); 7.09 (bs, 2H); 6.98 (s, 1H); 5.11 (s, 2H); 3.68 (s, 3H); 2.88 (m, 2H); 2.54 (m, 2H); 2.12 (m, 1H); 1.93 (m, 1H).

### Step 2

### Methyl 1-(5-hydroxy-4′-(trifluoromethyl)biphenyl-3-yl)cyclobutane carboxylate

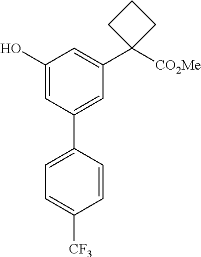

Pd/C (150 mg) was slowly added to a stirred solution of methyl 145-(benzyloxy)-4′-(trifluoromethyl)biphenyl-3-yl)cyclo-butanecarboxylate (1.5 g, 3.40 mmol) in MeOH (100 mL) under an atmosphere of nitrogen. The mixture was hydrogenated for 1.5 hs, upon which After the reaction mixture was filtered through a pad of Celite™ washing with MeOH. The volatiles were removed in vacuo to give methyl 1-(5-hydroxy-4′-(trifluoromethyl)biphenyl-3-yl)cyclobutane carboxylate in 92% yield. (1.09 g). ¹HNMR (CDCl3): 7.69 (s, 4H); 7.08 (s, 1H); 6.94 (s, 1H); 6.83 (s, 1H); 5.27 (bs, 1H); 3.68 (s, 3H); 2.87 (m, 2H); 2.56 (m, 2H); 2.08 (m, 1H); 1.92 (m, 1H).

### Step 3

### 1-(5-(2,2,2-trifluoroethoxy)-4′-(trifluoromethyl)biphenyl-3-yl)cyclo butane carboxylic acid

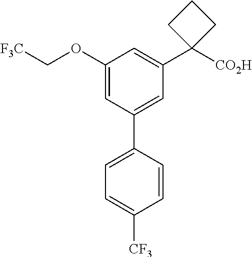

To a stirred mixture of methyl 1-(5-hydroxy-4′-(trifluoromethyl)biphenyl-3-yl)cyclobutane carboxylate (800 mg, 2.28 mmol) and K₂CO₃ (1.57 g, 11.37 mmol) in DMF (25 ml) was slowly added trifluoroethyl iodide (2.4 g, 11.42 mmol) at 0° C. over a period of 10 min. The mixture stirred for a further 30 min at 0° C. and then heated to 100° C. for 4 h. Upon completion of the reaction, the mixture was poured onto water and extracted with EtOAc (×2). The combined organic layers were washed with water, dried over Na₂SO₄ and concentrated in vacuo. The residue was purified by Flash Column Chromatography to give methyl 1-(5-(2,2,2-trifluoroethoxy)-4′-(trifluoromethyl)biphenyl-3-yl)cyclobutanecarboxylate in 45% yield. (444 mg). The ester (420 mg, 0.972 mmol) was dissolved in a MeOH/THF/Water mixture (10/ml/10 ml/5 ml) and lithium hydroxide monohydrate (12.2 mg, 2.916 mmol) was added. The mixture was stirred at RT for in for 1 h. Upon completion of the reaction, the volatiles were removed under reduced pressure, the residue was diluted with water, acidified with 5% HCl solution and extracted with EtOAc (×2). The combined organic layers were washed with water, dried with Na₂SO₄, filtered and concentrated in vacuo. The residue was purified by Flash Column Chromatography to give 1-(5-(2,2,2-trifluoroethoxy)-4′-(trifluoromethyl)biphenyl-3-yl)cyclo butane carboxylic acid in 52% yield. (211 mg). ¹HNMR (CDCl₃): 7.67 (s, 4H); 7.19 (s, 1H); 7.03 (s, 1H); 6.92 (s, 1H); 4.42 (q, 2H); 2.88 (m, 2H); 2.57 (m, 2H); 2.14 (m, 1H); 1.93 (m, 1H).

### Example 48

### 3-Cyclopropyl-2-(5-(2,2,2-trifluoroethoxy)-4′-(trifluoromethyl)biphenyl-3-yl) propanoic acid

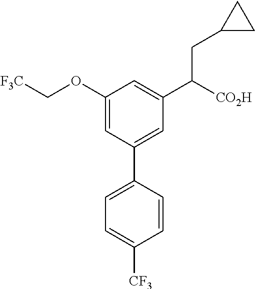

### Step 1

### Methyl 2-(5-(benzyloxy)-4′-(trifluoromethyl)biphenyl-3-yl)-3-cyclopropyl propanoate

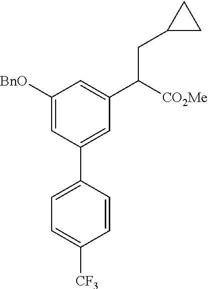

To a suspension of NaH (388 mg, 60% suspension, 16.5 mmol) in dry DMF (30 mL) was slowly added a mixture of methyl 2-(5-(benzyloxy)-4′-(trifluoromethyl)biphenyl-3-yl)acetate (4 g, 14.7 mmol) and cyclopropyl methyl bromide (1.54 mL, 16.5 mmol) at 0° C. under nitrogen atmosphere over a period of 15 min. The mixture was stirred for 30 min at 0° C., upon which the reaction mixture was poured onto crushed ice and extracted with EtOAc (×2). The combined organic layer were washed with water, dried over Na₂SO₄ and concentrated under reduced pressure. The residue was purified by flash column chromatography to give methyl 2-(5-(benzyloxy)-4′-(trifluoromethyl)biphenyl-3-yl)-3-cyclopropyl propanoate in 44% yield (2 g).

### Methyl 3-cyclopropyl-2-(5-hydroxy-4′-(trifluoromethyl)biphenyl-3-yl) propanoate

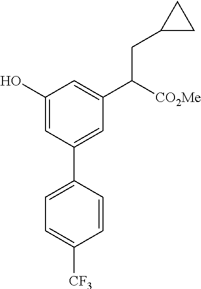

Pd (OH)₂ (500 mg) was slowly added to a stirred solution of methyl 2-(5-(benzyloxy)-4′-(trifluoromethyl)biphenyl-3-yl)-3-cyclopropyl propanoate (2 g) in 50 ml of methanol under an atmosphere of nitrogen. The reaction mixture was hydrogenated for 2 h. Upon completion the mixture was filtered through a pad of Celite™ washing with MeOH with methanol. The volatiles were evaporated under reduced pressure and the residue was purified by Flash column chromatography to give methyl 3-cyclopropyl-2-(5-hydroxy-4′-(trifluoromethyl)biphenyl-3-yl) propanoate in 62% yield (1 g). ¹HNMR (CDCl3, 200 MHz): 7.65 (m, 4H); 7.12 (s, 1H); 6.98 (s, 1H), 6.88 (s, 1H), 5.72 (bs, 1H), 3.72 (s, 3H), 3.62 (t, 1H), 1.84-1.98 (m, 2H); 0.65 (m, 1H), 0.42 (m, 2H), 0.11 (m, 2H).

### Methyl 3-cyclopropyl-2-(5-(2,2,2-trifluoroethoxy)-4′-(trifluoromethyl) biphenyl-3-yl)propanoate

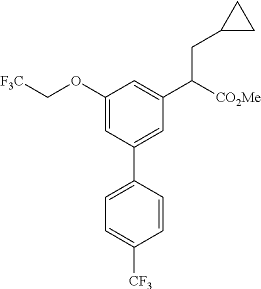

To a stirred mixture of methyl 3-cyclopropyl-2-(5-hydroxy-4′-(trifluoromethyl) biphenyl-3-yl) propanoate (300 mg, 1 eq) and potassium carbonate (240 mg, 1.8 eq) in 20 ml of DMF was slowly added trifluoroethyl iodide (0.16 ml, 2 eq) at 0° C. over a period of 10 min. The reaction mixture was stirred for 30 min at 0° C. and then heated at 100° C. for 4 h. Upon completion of the reaction, the mixture was poured into water and extracted with EtOAc (2×50 mL). The combined organic layers were washed with water, dried over Na₂SO₄ and evaporated under reduced pressure. The residue was purified by Flash Column Chromatography to give methyl 3-cyclopropyl-2-(5-(2,2,2-trifluoroethoxy)-4′-(trifluoromethyl)biphenyl-3-yl)propanoate in 60% yield (225 mg). ¹HNMR (CDCl3, 200 MHz): 7.65 (m, 4H); 7.22 (s, 1H); 7.05 (s, 1H), 6.98 (s, 1H), 4.4 (q, 2H), 3.76 (t, 1H), 3.68 (s, 3H), 1.84-1.98 (m, 2H); 0.65 (m, 1H), 0.44 (m, 2H), 0.11 (m, 2H).

### Step 2

### 3-Cyclopropyl-2-(5-(2,2,2-trifluoroethoxy)-4′-(trifluoromethyl)biphenyl-3-yl)propanoic acid

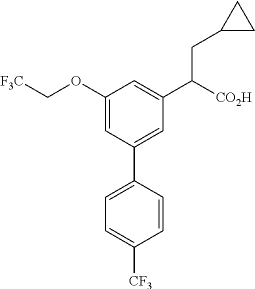

To a solution of compound methyl 3-cyclopropyl-2-(5-(2,2,2-trifluoroethoxy)-4′-(trifluoromethyl)biphenyl-3-yl)propanoate (220 mg, 1 eq) in a MeOH/THF/Water mixture (5 ml/5 ml/5 ml) was added lithium hydroxide monohydrate (118 mg, 6 eq). The reaction mixture was stirred for 2 h at RT. Upon completion of reaction, the volatiles were removed under reduced pressure. And the residue was diluted with water, acidified with 5% HCl solution and extracted with EtOAc (×2). The combined organic layers were washed with water, dried over Na₂SO₄, filtered and concentrated under reduced pressure. The crude product was purified by flash column chromatography to give compound 3-cyclopropyl-2-(5-(2,2,2-trifluoroethoxy)-4′-(trifluoromethyl)biphenyl-3-yl)propanoic acid in 97% yield (210 mg). ¹HNMR (CDCl3, 400 MHz): 7.71 (m, 4H); 7.25 (s, 1H); 7.05 (s, 1H), 6.98 (s, 1H), 4.41 (q, 2H), 3.75 (t, 1H), 1.84-1.98 (m, 2H); 0.65 (m, 1H), 0.44 (m, 2H), 0.11 (m, 2H).

### Methyl 2-(5-(benzyloxy)-4′-(trifluoromethyl)biphenyl-3-yl)-4-methyl pentanoate

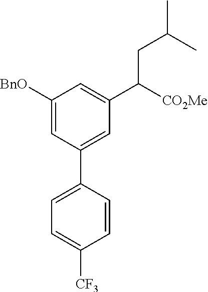

To a suspension of NaH (48 mg, 60% suspension, 2.1 mmol) in DMF was slowly added a mixture of methyl 2-(5-(benzyloxy)-4′-(trifluoromethyl)biphenyl-3-yl)acetate (400 mg, 1.0 mmol) and isobutyl bromide (0.12 mL, 2.1 mmol) DMF (10 mL) at 0° C. under an atmosphere of nitrogen over a period of 15 min. The mixture was and allowed to stir for another 15 min at 0° C., upon which it was poured onto crushed ice and extracted with ethyl acetate (2×10 mL). The combined organic layers were washed with water, dried over Na₂SO₄ and evaporated to give methyl 2-(5-(benzyloxy)-4′-(trifluoromethyl) biphenyl-3-yl)-4-methyl pentanoate (220 mg). ¹HNMR (CDCl₃): 7.68 (s, 4H); 7.42 (s, 5H); 7.15 (s, 1H); 7.14 (s, 1H); 7.08 (s, 1H); 5.13 (s, 2H); 3.72 (t, 1H); 3.69 (s, 3H); 2.02 (m, 1H); 1.71 (m, 1H); 1.48 (m, 1H); 0.93 (d, 6H).

### Methyl-2-(5-hydroxy-4′-(trifluoro ethyl) biphenyl-3-yl)-4-methyl pentanoate

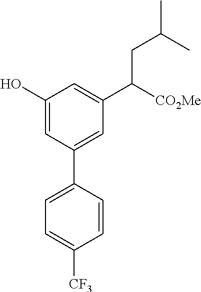

Pd(OH)₂ (80 mg) was slowly added to a stirred reaction mixture of methyl 245-(benzyloxy)-4′-(trifluoromethyl)biphenyl-3-yl)-4-methyl pentanoate (500 mg, 1.1 mmol) in MeOH (20 mL) under an of atmosphere nitrogen. The mixture was hydrogenated for 2 h, upon which the reaction catalyst was removed by filtration through a pad of Celite™ and washing with MeOH. The volatiles were evaporated from the filtrate to give methyl-2-(5-hydroxy-4′-(trifluoro ethyl) biphenyl-3-yl)-4-methyl pentanoate (350 mg) as oily liquid. ¹HNMR (CDCl₃): 7.66 (s, 4H); 7.12 (s, 1H); 6.97 (s, 1H); 6.87 (s, 1H); 4.98 (bs, 1H0; 3.68 (t, 1H); 3.67 (s, 3H); 2.02 (m, 1H); 1.98 (m, 1H); 1.70 (m, 1H); 0.94 (m, 1H); 0.92 (d, 6H).

### Example 17

### 2-(5-(ethoxy-4′-(trifluoromethyl)biphenyl-3-yl)-4-methylpentanoic acid

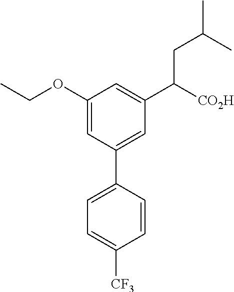

To a stirred mixture of methyl-2-(5-hydroxy-4′-(trifluoro ethyl) biphenyl-3-yl)-4-methyl pentanoate (500 mg, 1.3 mmol) and K₂CO₃ (0.361 g, 2.6 mmol) in DMF (25 ml) was slowly added ethyl iodide (0.408 g, 2.6 mmol) at 0° C. over a period of 10 min. The mixture was allowed to stir for another 30 min at 0° C. upon which it was heated at 60° C. for 4 h. After completion of the reaction, the mixture was poured into water and extracted with ethyl acetate (2×25 mL). The combined organic layers were washed with water, dried over Na₂SO₄, filtered and concentrated in vacuo. The residue was purified by flash column chromatography using (1:3 EtOAc:Hexane as eluent) to give methyl 2-(5-ethoxy-4′-(trifluoromethyl)biphenyl-3-yl)-4-methyl pentanoate (0.410 g).

A mixture of methyl 2-(5-ethoxy-4′-(trifluoromethyl)biphenyl-3-yl)-4-methyl pentanoate (390 mg, 0.95 mmol) and lithium hydroxide monohydrate (200 mg, 4.75 mmol) in a MeOH/THF/Water mixture (10 ml/10 ml/5 ml) was stirred at RT for 2 h. Upon completion of reaction, the volatiles were removed under reduced pressure, the residue was diluted with water, acidified with 5% HCl solution and extracted with ethyl acetate (2×25 mL). The combined organic layers were washed with water, dried over Na₂SO₄, filtered and evaporated. The residue was purified by Flash Column Chromatography (10% EtOAc/Hexane) to give 2-(5-(ethoxy-4′-(trifluoromethyl) biphenyl-3-yl)-4-methylpentanoic acid (300 mg) as an off white solid. ¹HNMR (CDCl₃, 500 MHz): 7.67 (m, 4H); 7.18 (s, 1H); 7.01 (s, 1H); 6.94 (s, 1H); 4.09 (q, 2H), 3.72 (t, 1H); 1.99 (m, 1H); 1.72 (m, 1H); 1.56 (m, 1H); 1.41 (t, 3H), 0.96 (d, 6H).

### Example 57

### 2-(5-(methoxyethoxy)-4′-(trifluoromethyl)biphenyl-3-yl)-4-methyl pentanoic acid

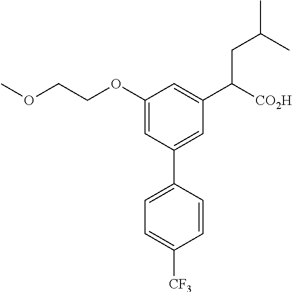

To a stirred mixture of methyl-2-(5-hydroxy-4′-(trifluoro ethyl) biphenyl-3-yl)-4-methyl pentanoate (500 mg, 1.3 mmol) and K₂CO₃ (0.361 g, 2.6 mmol) in DMF (25 mL) was slowly added 1-bromo-2-methoxyethane (0.45 g, 2.6 mmol) at 0° C. over a period of 10 min. The mixture was stirred for 30 min at 0° C. upon which it was heated at 60° C. for 4 h. The reaction mixture was poured into water and extracted with ethyl acetate (2×25 mL). The combined organic layers were washed with water, dried over Na₂SO₄, filtered and concentrated in vacuo. The residue was purified by Flash Column Chromatography using (1:4 EtOAc:Hexane as eluent) to give methyl 2-(5-(2-methoxyethoxy)-4′-(trifluoromethyl)biphenyl-3-yl)-4-methylpentanoate (356 mg).

A mixture of methyl 2-(5-(2-methoxyethoxy)-4′-(trifluoromethyl)biphenyl-3-yl)-4-methylpentanoate (200 mg, 0.4 mmol) and lithium hydroxide monohydrate (95 mg, 2.3 mmol) in MeOH/THF/Water mixture (10 ml/10 ml/5 ml) was stirred at RT for 2 h. Upon completion the reaction volatiles were removed under reduced pressure, the residue was diluted with water, acidified with 5% HCl solution and extracted with ethyl acetate (×2). The combined organic layers were washed with water, dried over Na₂SO₄, filtered and the voaltiles removed under reduced pressure. The residue was purified by Flash Column Chromatography (5% EtOAc:Hexane) to give 2-(5-(methoxyethoxy)-4′-(trifluoromethyl)biphenyl-3-yl)-4-methyl pentanoic acid (100 mg) as a colorless oil. ¹HNMR (CDCl₃): 7.67 (s, 4H); 7.23 (s, 1H); 7.14 (s, 1H); 6.97 (s, 1H); 4.42 (q, 2H); 3.75 (t, 1H); 2.03 (m, 1H); 1.72 (m, 1H); 1.56 (m, 1H); 0.96 (d, 6H).

### Example 7

### 2-(5-methoxy-4′-(trifluoromethyl)biphenyl-3-yl)-4-methylpentanoic acid

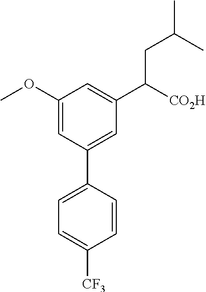

To a stirred mixture of methyl-2-(5-hydroxy-4′-(trifluoro ethyl) biphenyl-3-yl)-4-methyl pentanoate (500 mg, 1.3 mmol) and K₂CO₃ (360 mg, 2.6 mmol) in DMF (25 mL) was slowly added methyl iodide (420 mg, 2.6 mmol) at 0° C. over a period of 10 min. The reaction mixture was stirred for 30 min at 0° C. and then heated at 60° C. for 4 h. Upon completion of the reaction, the mixture was poured onto water and extracted with ethyl acetate (2×25 mL). The combined organic layers were washed with water, dried over Na₂SO₄, filtered and the volatiles removed under reduced pressure. The residue was purified by Flash Column Chromatography to give methyl 2-(5-methoxy-4′-(trifluoromethyl)biphenyl-3-yl)-4-methylpentanoate (300 mg).

A mixture of methyl 2-(5-methoxy-4′-(trifluoromethyl)biphenyl-3-yl)-4-methylpentanoate (300 mg, 1.52 mmol) and lithium hydroxide monohydrate (160 mg, 3.8 mmol) in MeOH/THF/Water mixture (10 ml/10 ml/10 ml) was stirred for 2 h at RT. Upon completion of the reaction, the volatiles were removed under reduced pressure, the residue was diluted with water, acidified with 5% HCl solution and extracted with ethyl acetate (×2). The combined organic layers were washed with water, dried with Na₂SO₄, filtered and concentrated in vacuo. The residue was purified by Flash Column Chromatography (5% EtOAc/Hexane) to give 2-(5-methoxy-4′-(trifluoromethyl) biphenyl-3-yl)-4-methylpentanoic acid (240 mg) as an off white solid. ¹HNMR (CDCl₃, 500 MHz): 7.67 (m, 4H); 7.18 (s, 1H); 7.01 (s, 1H); 6.94 (s, 1H); 3.88 (s, 3H); 3.72 (t, 1H); 1.99 (m, 1H); 1.72 (m, 1H); 1.56 (m, 1H); 0.96 (d, 6H).

### Example 1936

### 2-(5-(benzo[c][1,2,5]thiadiazol-5-yl methoxy)-4′-(trifluoromethyl) biphenyl-3-yl)pentanoic acid

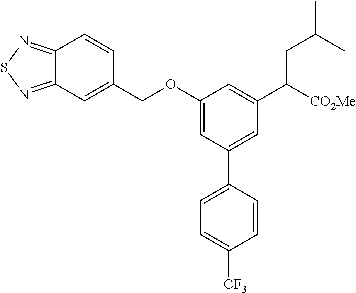

To a stirred mixture of methyl-2-(5-hydroxy-4′-(trifluoro ethyl) biphenyl-3-yl)-4-methyl pentanoate (110 mg, 0.3 mmol) and cesium carbonate (267 mg, 0.81 mmol) in dry DMF (25 mL) was slowly added thiadiazole methyl bromide (139 mg, 0.54 mmol) at 0° C. over a period of 10 min. The reaction mixture was stirred for 30 min at 0° C. and then heated at 100° C. for 4 h. Upon completion of the reaction, the mixture was poured into water and extracted with ethyl acetate (2×25 mL). The combined organic layers were washed with water, dried over Na₂SO₄, filtered and concentrated under reduced pressure. The residue was purified by Flash Column Chromatography using (1:4 EtOAC: Hexane as eluent) to methyl 2-(5-(benzo[c][1,2,5]thiadiazol-5-ylmethoxy)-4′-(trifluoromethyl)biphenyl-3-yl)-4-methyl pentanoate (70 mg).

A mixture of methyl 2-(5-(benzo[c][1,2,5]thiadiazol-5-ylmethoxy)-4′-(trifluoromethyl)biphenyl-3-yl)-4-methyl pentanoate (140 mg, 0.28 mmol) and lithium hydroxide monohydrate (122 mg, 2.9 mmol) in a MeOH/THF/Water solvent mixture (10 ml/10 ml/5 ml) was stirred at RT for 2 h. Upon completion of the reaction, the volatiles were removed under reduced pressure, the residue was diluted with water, acidified with 5% HCl solution and extracted with ethyl acetate (2×25 mL). The combined organic layers were washed with water, dried over Na₂SO₄, filtered and concentrated under reduced pressure. The residue was purified by flash column chromatography (using 5% EtOAc/Hexane) to give 2-(5-(benzo[c][1,2,5]thiadiazol-5-yl methoxy)-4′-(trifluoromethyl)biphenyl-3-yl)pentanoic acid (100 mg) as a white solid. ¹HNMR (CDCl₃, 500 MHz): 8.1 (s, 1H), 8.03 (d, 1H), 7.66 (m, 4H); 7.17 (s, 1H); 7.12 (s, 1H); 7.04 (s, 1H); 5.3 (s, 2H), 3.72 (t, 1H); 2.02 (m, 1H); 1.72 (m, 1H); 1.56 (m, 1H); 0.96 (d, 6H).

### Example 1906

### 3-Cyclopropyl-2-(5-(2,2,2-trifluoroethoxy)-4′-(trifluoromethyl)biphenyl-3-yl) propanoic acid

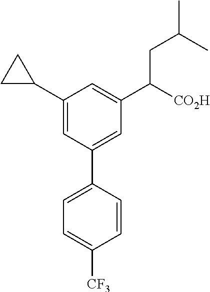

### Step 1

### Methyl-2-(5-cyclopropyl-4′-(trifluoromethyl)biphenyl-3-yl)-4-methyl pentanoate

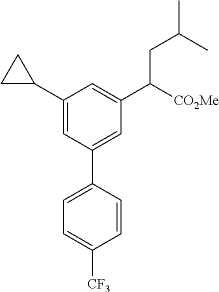

To a stirred solution of methyl-2-(5-hydroxy-4′-(trifluoromethyl)biphenyl-3-yl)-4-methyl pentanoate (320 mg, 1.0 mmol) in dry DCM (50 mL) was slowly added DIPEA (0.22 mL, 1.3 mmol) at 0° C. followed by Triflic anhydride (0.197 mL, 1.2 mmol). The reaction mixture was stirred at 0° C. for 30 mins. Upon completion of the reaction, the mixture was poured onto crushed ice and extracted with methylene chloride (2×50 mL). The combined organic layers were washed with 10% NaHCO₃ solution followed by water. The organic layer was dried over Na₂SO₄, filtered and evaporated under reduced pressure. The residue (total 400 mg) was taken as such for the next step without further purification. A mixture of the crude triflate (300 mg, 0.6 mmol), cyclopropyl boronic acid (155 mg, 1.8 mmol), palladium (II) (42 mg, 0.06 mmol), cesium carbonate (883 mg, 2.7 mmol) in 1,4-dioxane:H₂O (20 ml:1 mL) was stirred for 4 h at 100° C. Upon completion of the reaction, the solids were removed by filtration. The filtrate was diluted with water and extracted with ethyl acetate (2×50 mL). The combined organic layers were washed with water followed by brine, dried over Na₂SO₄ and concentrated under reduced pressure. The esidue was purified by flash column chromatography (using 10 EtOAC/Hexane) to give methyl-2-(5-cyclopropyl-4′-(trifluoromethyl) biphenyl-3-yl)-4-methyl pentanoate (100 mg, 48% yield) as a thick oily liquid.

### Step 2

### 3-Cyclopropyl-2-(5-(2,2,2-trifluoroethoxy)-4′-(trifluoromethyl)biphenyl-3-yl)propanoic acid

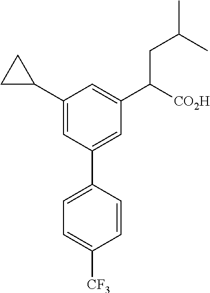

A solution of compound methyl-2-(5-cyclopropyl-4′-(trifluoromethyl)biphenyl-3-yl)-4-methyl pentanoate (100 mg, 0.29 mmol) and lithium hydroxide monohydrate (61 mg, 1.4 mmol) in a MeOH/THF/Water mixture (5 ml/5 ml/5 ml) was stirred at for 2 h at RT. Upon completion of the reaction, the volatiles were removed under reduced pressure the residue was diluted with water, acidified with 5% HCl solution and extracted with ethyl acetate (×2). The combined organic layers were washed with water, dried over Na₂SO₄, filtered and evaporated under reduced pressure. The residue was purified by flash column chromatography (1:1 EtOA/Hexane) to give compound 3-Cyclopropyl-2-(5-(2,2,2-trifluoroethoxy)-4′-(trifluoromethyl)biphenyl-3-yl) propanoic acid (25 mg) as white solid. ¹HNMR (CDCl₃, 400 MHz): 7.66 (m, 4H); 7.32 (s, 1H); 7.14 (s, 1H), 7.06 (s, 1H), 3.7 (t, 1H), 1.94-1.99 (m, 2H); 1.5-1.74 (m, 2H), 0.71-1.02 (m, 8H).

### Methyl-2-(3-(benzyloxy)-5-(2,2,2-trifluoroethoxy)-phenyl)acetate

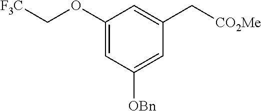

To a stirred mixture of methyl 2-(3-(benzyloxy)-5-hydroxyphenyl)acetate (500 mg, 1.8 mmol), potassium carbonate (500 mg, 3.6 mmol) in DMF (20 mL) was slowly added trifluoroethyl iodide (1.08 ml, 0.11 mmol) at 0° C. over a period of 10 min. The reaction mixture was stirred for a further 30 min at 0° C. and then heated to 100° C. for 4 h. Upon completion of the reaction, the mixture was poured into water and extracted with ethyl acetate (2×50 mL). The combined organic layers were washed with water, dried over Na₂SO₄ and evaporated under reduced pressure. The residue was purified by flash column chromatography using (1:4 EtOAc:Hexane as eluent) to give methyl-2-(3-(benzyloxy)-5-(2,2,2-trifluoroethoxy)-phenyl)acetate (225 mg) as an oil.

### Methyl 2-(3-(benzyloxy)-5-(2,2,2-trifluoroethoxy)phenyl)-3-cyclopropylpropanoate

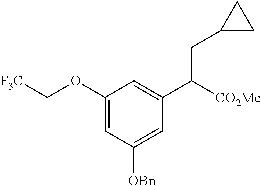

To a suspension of NaH (275 mg, 60% suspension, 10.4 mmol) in dry DMF (30 mL) was slowly added a mixture of methyl-2-(3-(benzyloxy)-5-(2,2,2-trifluoroethoxy)-phenyl)acetate (3.7 g, 10.4 mmol) and cyclopropyl methyl bromide (1.2 mL, 12.5 mmol) at 0° C. under an nitrogen atmosphere over a period of 15 min. The mixture was stirred for 30 min at 0° C., upon which the mixture was poured onto crushed ice and extracted with ethyl acetate (2×50 mL). The combined organic layers were washed with water, dried over Na2SO4 and evaporated under reduced pressure. The residue was purified by column chromatography using (1:4 EtOAc:Hexane as eluent) to yield methyl 2-(3-(benzyloxy)-5-(2,2,2-trifluoroethoxy)phenyl)-3-cyclopropylpropanoate (2.5 g) as an oil.

### Methyl 3-cyclopropyl-2-(3-hydroxy-5-(2,2,2-trifluoroethoxy)phenyl)propanoate

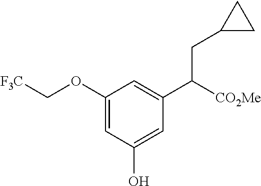

Pd/C (500 mg) was slowly added to a stirred solution of methyl 2-(3-(benzyloxy)-5-(2,2,2-trifluoroethoxy)phenyl)-3-cyclopropylpropanoate (2 g) in methanol (MeOH) under an atmosphere of nitrogen. The mixture was hydrogenated for 2 h, upon which the mixture was filtered through a bed of Celite™ washing with methanol. The volatiles were removed under reduced pressure and the residue was purified by Flash column chromatography to give methyl 3-cyclopropyl-2-(3-hydroxy-5-(2,2,2-trifluoroethoxy)phenyl) propanoate (1 g). ¹HNMR (CDCl3, 200 MHz): 7.65 (m, 4H); 7.12 (s, 1H); 6.98 (s, 1H), 6.88 (s, 1H), 5.72 (bs, 1H), 3.72 (s, 3H), 3.62 (t, 1H), 1.84-1.98 (m, 2H); 0.65 (m, 1H), 0.42 (m, 2H), 0.11 (m, 2H).

### Example 1628

### 2-(3-(Benzo[c][1,2,5]oxadiazol-5-yl)-5-(2,2,2-trifluoroethoxy)phenyl)-3-cyclopropylpropanoic acid

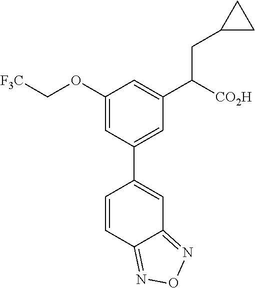

### Step 1

### Methyl 2-(3-(benzo[c][1,2,5]oxadiazol-5-yl)-5-(2,2,2-trifluoroethoxy)phenyl)-3-cyclopropylpropanoate

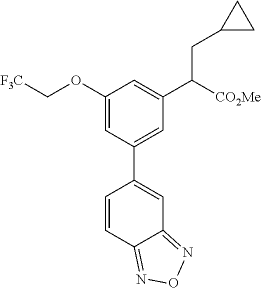

To a stirred solution of methyl 3-cyclopropyl-2-(3-hydroxy-5-(2,2,2-trifluoroethoxy)phenyl) propanoate (200 mg, 0.62 mmol) in dry DCM (20 mL) was slowly added DIPEA (0.142 mL, 0.81 mmol) at 0° C. followed by triflic anhydride (0.12 mL, 0.74 mmol). The reaction mixture was stirred for another 30 min at 0° C. Upon completion of the reaction, the mixture was poured onto crushed ice and extracted with methylene dichloride (2×50 mL). The combined organic layers were washed with 10% NaHCO₃ solution followed by water. The organic layer was dried over Na₂SO₄, filtered and evaporated to give the corresponding triflate (350 mg) which was taken as into next step without further purification. A mixture of the triflate (350 mg, 0.77 mmol), benzo[c][1,2,5]oxadiazol-5-ylboronic acid (287 mg, 1.16 mmol), palladium (II) (63 mg, 0.07 mmol), cesium carbonate (1.14 g, 3.5 mmol) in 1,4-dioxane (25 mL) was stirred for 3 h at 100° C. Upon completion of the reaction, the solids were removed by filtration. The filtrate was diluted with water and extracted with ethyl acetate (2×100 mL). The combined organic layers were washed with water followed by brine, dried over Na₂SO₄ and concentrated under reduced pressure. The residue was purified by flash column chromatography using (1:4 EtOAc:Hexane as eluent) to give methyl 2-(3-(benzo[c][1,2,5]oxadiazol-5-yl)-5-(2,2,2-trifluoroethoxy)phenyl)-3-cyclopropylpropanoate (320 mg) in 78% yield.

### Step 2

### 2-(3-(Benzo[c][1,2,5]oxadiazol-5-yl)-5-(2,2,2-trifluoroethoxy)phenyl)-3-cyclopropylpropanoic acid

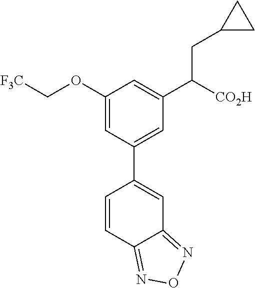

A solution of 2-(3-(benzo[c][1,2,5]oxadiazol-5-yl)-5-(2,2,2-trifluoroethoxy)phenyl)-3-cyclopropylpropanoate (320 mg, 0.76 mmol) and lithium hydroxide monohydrate (191 mg, 4.5 mmol) in a MeOH/THF/Water mixture (10 ml/10 ml/5 ml) was stirred at RT for 2 h. Upon completion of the reaction, the volatiles were removed under reduced pressure. The residue was diluted with water, acidified with 5% HCl solution and extracted with ethyl acetate (×2). The combined organic layers were washed with water, dried over Na2SO4, filtered and evaporated under reduced pressure. The residue was purified by flash column chromatography using (1:3 EtOAc:Hexane as eluent) to give compound 2-(3-(Benzyo[c][1,2,5]oxadiazol-5-yl)-5-(2,2,2-trifluoroethoxy)phenyl)-3-cyclopropylpropanoic acid (180 mg). ¹HNMR (CDCl3, 400 MHz): 8.18 (s, 1H), 8.06 (d, 1H), 7.82 (d, 1H); 7.37 (s, 1H); 7.19 (s, 1H), 7.02 (s, 1H), 4.42 (q, 2H), 3.79 (t, 1H), 1.84-1.98 (m, 2H); 0.68 (m, 1H), 0.44 (m, 2H), 0.05-0.11 (m, 2H).

### Example 1638

### 2-(3-(Benzo[c][1,2,5]thiadiazol-5-yl)-5-(2,2,2-trifluoroethoxy)phenyl)-3-cyclopropyl propanoate

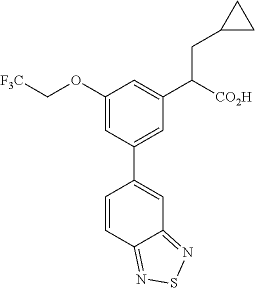

### Step 1

### Methyl 2-(3-(benzo[c][1,2,5]thiadiazol-5-yl)-5-(2,2,2-trifluoroethoxy)phenyl)-3-cyclopropylpropanoate

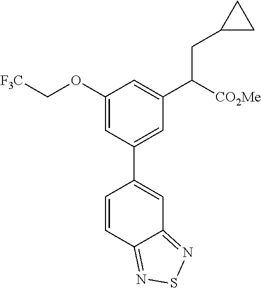

To a stirred solution of methyl 3-cyclopropyl-2-(3-hydroxy-5-(2,2,2-trifluoroethoxy)phenyl) propanoate (200 mg, 0.62 mmol) in dry DCM (20 mL) was slowly added DIPEA (0.142 mL, 0.81 mmol) at 0° C. followed by triflic anhydride (0.12 mL, 0.74 mmol). The reaction mixture was stirred for another 30 min at 0° C. Upon completion of the reaction, the mixture was poured onto crushed ice and extracted with methylene dichloride (2×50 mL). The combined organic layers were washed with 10% NaHCO₃ solution followed by water, dried over Na₂SO₄, filtered and concentrated under reduced pressure to give the corresponding triflate (350 mg). The trilfate was used in the next step without further purification. A mixture of the triflate (350 mg, 0.77 mmol), benzo[c][1,2,5]thiadiazol-5-ylboronic acid (287 mg, 1.16 mmol), palladium (II) (63 mg, 0.07 mmol), cesium carbonate (1.14 g, 3.5 mmol) in 1,4-dioxane (25 mL) was stirred for 3 h at 100° C. Upon completion of the reaction, the solids were removed by filtration, the filtrate was diluted with water and extracted with ethyl acetate (2×100 mL). The combined organic layers were washed with water followed by brine, dried over Na₂SO₄ and concentrated under reduced pressure. The esidue was purified by flash column chromatography to give methyl 2-(3-(benzo[c][1,2,5]thiadiazol-5-yl)-5-(2,2,2-trifluoroethoxy)phenyl)-3-cyclopropylpropanoate (320 mg).

### Step 2

### 2-(3-(Benzo[c][1,2,5]thiadiazol-5-yl)-5-(2,2,2-trifluoroethoxy)phenyl)-3-cyclopropyl propanoate

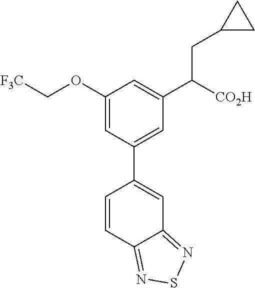

A solution of methyl 2-(3-(benzo[c][1,2,5]thiadiazol-5-yl)-5-(2,2,2-trifluoroethoxy)phenyl)-3-cyclopropylpropanoate (320 mg, 0.76 mmol) and lithium hydroxide monohydrate (191 mg, 4.5 mmol) in MeOH/THF/Water mixture (10 ml/10 ml/5 ml) were stirred at RT for 2 h. Upon completion of the reaction, the volatiles were removed under reduced pressure and the residue was diluted with water, acidified with 5% HCl solution and extracted with ethyl acetate (×2). The combined organic layers were washed with water, dried over Na₂SO₄, filtered and concentrated under reduced pressure. The residue was purified by flash column chromatography using (1:4 EtOAc:Hexane as eluent) to give compound 2-(3-(benzo[c][1,2,5]thiadiazol-5-yl)-5-(2,2,2-trifluoroethoxy)phenyl)-3-cyclopropyl propanoate (180 mg). ¹HNMR (CDCl3, 400 MHz): 12.4 (bs, 1H), 8.4 (s, 1H), 8.18 (d, 1H), 8.01 (d, 1H), 7.48 (m, 2H); 7.12 (s, 1H); 4.92 (m, 2H), 4.41 (q, 2H), 3.75 (t, 1H), 1.84-1.98 (m, 2H); 0.65 (m, 1H), 0.44 (m, 2H), 0.05-0.11 (m, 2H).

### Example 108

### 2-(4′-Chloro-5-(2,2,2-trifluoroethoxy)biphenyl-3-yl)-3-cyclopropylpropanoic acid

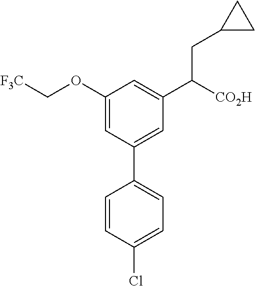

### Step 1

### Methyl 2-(4′-chloro-5-(2,2,2-trifluoroethoxy)biphenyl-3-yl)-3-cyclopropylpropanoate

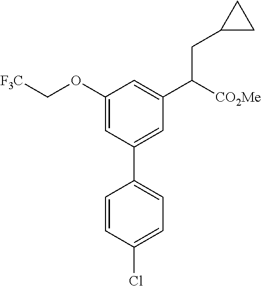

A mixture of methyl 3-cyclopropyl-2-(3-(2,2,2-trifluoroethoxy)-5-(trifluoromethylsulfonyloxy)phenyl) propanoate (see examples 1628 and 1638 for synthetic procedure (500 mg, 1.1 mmol), 4-chlorophenylboronic acid (308 mg, 2.1 mmol), palladium (II) (78 mg, 0.1 mmol), cesium carbonate (1.49 g, 4.8 mmol) in 1,4-dioxane:H₂O (50 ml:10 mL) was stirred for 4 h at 100° C. Upon completion of the reaction, the solids were removed by filtration. The filtrate was diluted with water and extracted with ethyl acetate (2×100 mL). The combined organic layers were washed with water followed by brine, dried over Na₂SO₄ and concentrated under reduced pressure. The residue was purified by flash column chromatography using (1:4 EtOAc:Hexane as eluent) to give methyl 2-(4′-chloro-5-(2,2,2-trifluoroethoxy)biphenyl-3-yl)-3-cyclopropylpropanoate (220 mg).

### Step 2

### 2-(4′-Chloro-5-(2,2,2-trifluoroethoxy)biphenyl-3-yl)-3-cyclopropylpropanoic acid

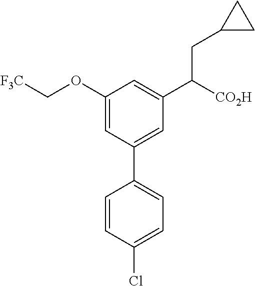

A solution of compound methyl 2-(4′-chloro-5-(2,2,2-trifluoroethoxy)biphenyl-3-yl)-3-cyclopropylpropanoate (220 mg, 0.6 mmol) and lithium hydroxide monohydrate (209 mg, 4.9 mmol) in a MeOH/THF/H₂O mixture (5 ml/5 ml/5 ml) was stirred at RT for 2 h.

After completion of the reaction, the volatiles were removed under reduced pressure. The residue was diluted with water, acidified with 5% HCl solution and extracted with ethyl acetate (×2). The combined organic layers were washed with water, dried over Na₂SO₄, filtered and concentrated under reduced pressure. The residue was purified by flash column chromatography using (1:3 EtOAc:Hexane as eluent) to give compound 2-(4′-Chloro-5-(2,2,2-trifluoroethoxy)biphenyl-3-yl)-3-cyclopropylpropanoic acid (180 mg). ¹HNMR (CDClₛ, 400 MHz): 7.58 (d, 2H); 7.42 (d, 2H); 7.25 (s, 1H), 7.05 (s, 1H), 6.96 (s, 1H), 4.41 (q, 2H), 3.75 (t, 1H), 1.98 (m, 1H); 1.82 (m, 1H), 0.68 (m, 1H), 0.44 (m, 2H), 0.11 (m, 2H).

### Example 168

### Methyl 3-cyclopropyl-2-(4′-fluoro-5-(2,2,2-trifluoroethoxy)biphenyl-3-yl)propanoate

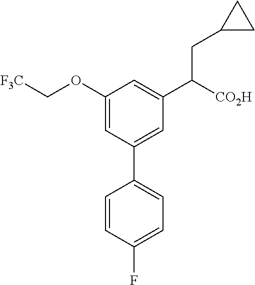

### Step 1

### Methyl-3-cyclopropyl-2-(4′-fluoro-5-(2,2,2-trifluoroethoxy) biphenyl-3-yl)propanoate

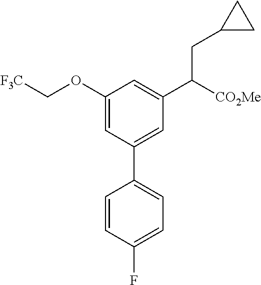

A mixture of methyl 3-cyclopropyl-2-(3-(2,2,2-trifluoroethoxy)-5-(trifluoromethylsulfonyloxy)phenyl)propanoate (500 mg, 1.1 mmol), 4-fluorophenylboronic acid (308 mg, 2.2 mmol), palladium (II) (78 mg, 0.1 mmol), cesium carbonate (1.6 g, 4.9 mmol) in 1,4-dioxane:H₂O (50 ml:10 mL) was stirred for 4 h at 100° C. Upon completion of the reaction, the solids were removed by filtration. The filtrate was diluted with water and extracted with ethyl acetate (2×100 mL). The combined organic layers were washed with water followed by brine, dried over Na₂SO₄ and concentrated under reduced pressure. The residue was purified by flash column chromatography using (1:4 EtOAc:Hexane as eluent) to give methyl-3-cyclopropyl-2-(4′-fluoro-5-(2,2,2-trifluoroethoxy) biphenyl-3-yl) propanoate (300 mg). ¹HNMR (CDCl₃, 200 MHz): 7.68 (m, 2H); 7.44 (m, 2H); 7.35 (s, 1H), 5.15 (s, 2H), 3.75 (s, 3H), 3.64 (s, 2H).

### 3-cyclopropyl-2-(4′-fluoro-5-(2,2,2-trifluoroethoxy) biphenyl-3-yl)propanoic acid

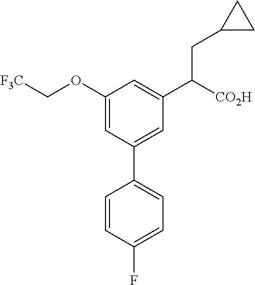

A solution of compound methyl-3-cyclopropyl-2-(4′-fluoro-5-(2,2,2-trifluoroethoxy) biphenyl-3-yl) propanoate (300 mg, 0.76 mmol) and lithium hydroxide monohydrate (255 mg, 6.09 mmol) in a MeOH/THF/H₂O mixture (5 ml/5 ml/5 ml) was stirred at RT for 2 h. Upon completion of the reaction, the volatiles were removed under reduced pressure. The residue was diluted with water, acidified with 5% HCl solution and extracted with ethyl acetate (×2). The combined organic layers were washed with water, dried over Na₂SO₄, filtered and concentrated under reduced pressure. The mixtures was purified by flash column chromatography using (1:3 EtOAc:Hexane as eluent) to give compound 3-cyclopropyl-2-(4′-fluoro-5-(2,2,2-trifluoroethoxy) biphenyl-3-yl) propanoic acid (212 mg). ¹HNMR (CDCl₃, 400 MHz): 7.71 (m, 4H); 7.25 (s, 1H); 7.05 (s, 1H), 6.98 (s, 1H), 4.41 (q, 2H), 3.75 (t, 1H), 1.84-1.98 (m, 2H); 0.65 (m, 1H), 0.44 (m, 2H), 0.05-0.15 (m, 2H).

### (3-Bromo-5-(4-(trifluoromethyl)benzyloxy)phenyl)methanol

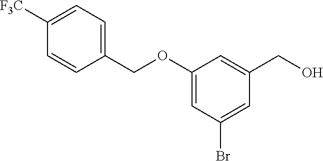

To a stirred solution of 3-bromo-5-(hydroxymethyl)phenol (9 g, 44 mmol) in DMSO (50 mL), K₂CO₃ (9.17 g, 66 mmol) was added slowly at room temperature. The reaction mixture was cooled to 0° C. and p-CF₃-benzyl bromide (11.6 g, 48 mmol) was added slowly over a period of 15 min under an atmosphere of nitrogen. Upon completion of the addition, the reaction mixture was allowed to warm room temperature and stirred for 8 h. The eaction mixture was filtered through small pad of Celite™ pad and the filtrate was concentrated under reduced pressure. The residue was purified by Flash column Chromatography (1:4 EtOAc/Hexane as eluent) to give (3-bromo-5-(4-(trifluoromethyl)benzyloxy)phenyl)methanol (7 g).

### 3-Bromo-5-(4-(trifluoromethyl)benzyloxy)benzyl methanesulfonate

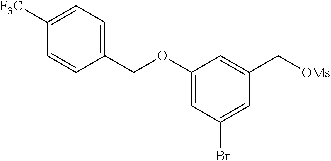

To a stirred solution of (3-bromo-5-(4-(trifluoromethyl)benzyloxy)phenyl)methanol (7 g, 19 mmol) in dry DCM (50 mL) was slowly added triethyl amine (3.91 g, 38 mmol) at 0° C. over 10 mi., followed by methane sulfonyl chloride (2.6 g, 23 mmol). The reaction mixture was stirred for further 2 h 0° C. Upon completion of the reaction, the mixture was poured into water and extracted with dichloromethane (2×50 mL). The combined organic layers were washed with water, dried over Na₂SO₄ and concentrated under reduced pressure. The residue was purified by flash column chromatography (20% EtOAc/Hexane as eluent) to give 3-bromo-5-(4-(trifluoromethyl)benzyloxy)benzyl methanesulfonate (8 g) as a liquid.

### 2-(3-Bromo-5-(4-(trifluoromethyl)benzyloxy)phenyl acetonitrile

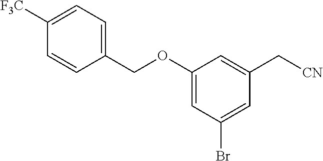

A mixture of 3-bromo-5-(4-(trifluoromethyl)benzyloxy)benzyl methanesulfonate (8 g, 18 mmol), sodium cyanide (1.07 g, 21 mmol) in acetonitrile: water (50 mL: 10 mL), tetrabutyl ammonium bromide (1.17 g, 3.6 mmol) was stirred at 80° C. for 8 h. Upon completion of the reaction, the mixture was poured into water and extracted with ethyl acetate (2×50 mL). The combined organic layers were washed with water, dried over Na₂SO₄ and concentrated under reduced pressure. The residue was purified by flash column chromatography (10% EtOAc/Hexane) to give 2-(3-bromo-5-(4-(trifluoromethyl)benzyloxy)phenyl acetonitrile (6.5 g) as an oil.

### Ethyl-2-(3-bromo-5-(4-(trifluoromethyl)benzyloxy)phenyl acetate

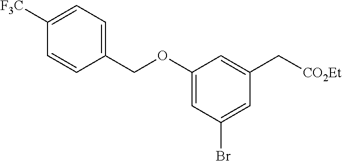

A solution of 2-(3-bromo-5-(4-(trifluoromethyl)benzyloxy)phenyl acetonitrile (6.5 g, 17.5 mmol) in ethanolic HCl (100 mL, 20% solution), was stirred for 30 min at rt and then heated at 60° C. overnight. Upon completion of the reaction, the volatiles were removed under reduced pressure and the residue was diluted with water and extracted with ethyl acetate (2×100 mL). The combined organic layers were washed with NaHCO₃ solution, water, dried over Na₂SO₄ and concentrated under reduced pressure. The residue was purified by flash column chromatography (10% EtOAc/Hexane as eluent) to give ethyl-2-(3-bromo-5-(4-(trifluoromethyl)benzyloxy)phenyl acetate (6.5 g) as an oil.

### Ethyl-2-(3-bromo-5-(4-(trifluoromethyl)benzyloxy)phenyl)-4-methyl pentanoate

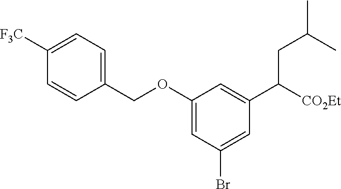

To a suspension of NaH (434 mg, 60% suspension, 18 mmol) in dry DMF (20 mL) was slowly added a mixture of ethyl-2-(3-bromo-5-(4-(trifluoromethyl)benzyloxy)phenyl acetate (3.6 g, 8.6 mmol) and isobutyl bromide (1.24 g, 9.0 mmol) at 0° C. under an atmosphere of nitrogen over a period of 15 min The mixture was allowed to be stirred at 0° C. for 30 min to complete the reaction. The mixture was poured onto crushed ice and extracted with ethyl acetate (2×50 mL). The combined organic layers were washed with water, dried over Na₂SO₄ and concentrated under reduced pressure. The residue was purified by column chromatography (using 5% EtOAc/Hexane) to yield ethyl-2-(3-bromo-5-(4-(trifluoromethyl)benzyloxy)phenyl)-4-methyl pentanoate (3.5 g) as an oil.

### Example 1587

### 2-(3-(Benzo[c][1,2,5]oxadiazol-5-yl)-5-(4-(trifluoromethyl)benzyloxy)phenyl)-4-methylpentanoic acid

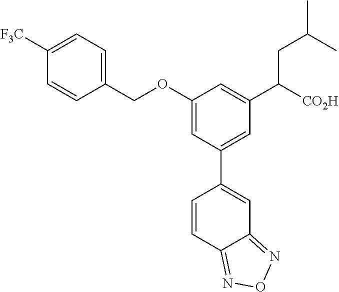

### Ethyl-2-(3-benzo[c][1,2,5]oxadiazol-5-yl)-5-(4-(trifluoromethyl)benzyloxy)phenyl)-4-methyl pentanoate

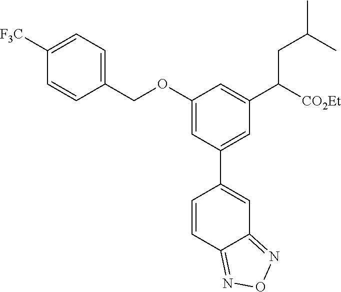

A mixture of ethyl-2-(3-bromo-5-(4-(trifluoromethyl)benzyloxy)phenyl)-4-methyl pentanoate (500 mg, 1.05 mmol), benzo[c][1,2,5]oxadiazol-5-ylboronic acid (285 mg, 1.1 mmol), tetrakis(triphenyl phosphene) palladium (0) (244 mg, 0.21 mmol), cesium carbonate (1.2 g, 3.69 mmol) in DMF: H₂O (30 ml:10 mL) was stirred for 8 h at 80° C. Upon completion of the reaction, the solids were removed by filtration. The filtrate was diluted with water and extracted with ethyl acetate (2×100 mL). The combined organic layers were washed with water followed by brine, dried over Na₂SO₄ and concentrated under reduced pressure. The residue was purified by flash column chromatography (using 10% EtOAc/Hexane as eluent) to give ethyl-2-(3-benzo[c][1,2,5]oxadiazol-5-yl)-5-(4-(trifluoromethyl)benzyloxy)phenyl)-4-methyl pentanoate (150 mg) as an oil.

### 2-(3-benzo[c][1,2,5]oxadiazol-5-yl)-5-(4-(trifluoromethyl)benzyloxy)phenyl)-4-methyl pentanoic acid

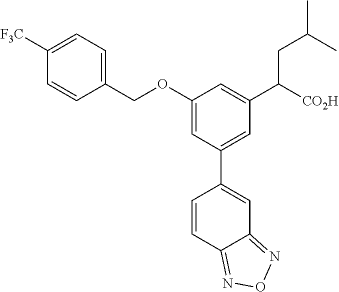

A solution of ethyl-2-(3-benzo[c][1,2,5]oxadiazol-5-yl)-5-(4-(trifluoromethyl)benzyloxy)phenyl)-4-methyl pentanoate (150 mg, 0.29 mmol), in MeOH/THF/H₂O mixture (10 ml/10 ml/5 ml) and lithium hydroxide monohydrate (61 mg, 1.4 mmol) were stirred at RT for 2 h. Upon completion of the reaction, the volatiles were removed under reduced pressure and the residue was diluted with water, acidified with 5% HCl solution and extracted with ethyl acetate (×2). The combined organic layers were washed with water, dried over Na₂SO₄, filtered and concentrated under reduced pressure. The residue was purified by flash column chromatography (using 1:1 EtOAc/Hexane as eluent) to give compound 2-(3-benzo[c][1,2,5]oxadiazol-5-yl)-5-(4-(trifluoromethyl)benzyloxy)phenyl)-4-methyl pentanoic acid (40 mg). ¹HNMR (CDCl₃, 400 MHz): 7.96 (d, 2H); 7.68 (m, 3H), 7.59 (d, 2H); 7.21 (s, 1H), 7.15 (s, 1H), 7.04 (s, 1H), 5.2 (s, 2H), 3.75 (t, 1H), 1.99 (m, 1H); 1.74 (m, 1H), 1.52 (m, 1H), 0.94 (d, 6H).

### Example 1597

### 2-(3-(Benzo[c][1,2,5]thiadiazol-5-yl)-5-(4-(trifluoromethyl)benzyloxy)phenyl)-4-methylpentanoic acid

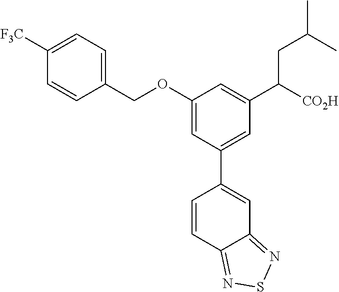

### Step 1

### Ethyl-2-(3-benzo[c][1,2,5]thiadiazol-5-yl)-5-(4-(trifluoromethyl)benzyloxy)phenyl)-4-methyl pentanoate

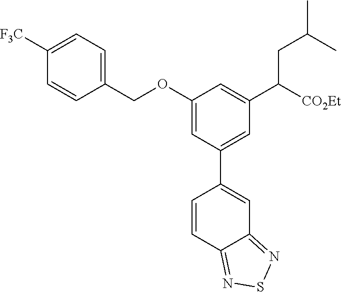

A mixture of ethyl-2-(3-bromo-5-(4-(trifluoromethyl)benzyloxy)phenyl)-4-methyl pentanoate (500 mg, 1.05 mmol), benzo[c][1,2,5]thiadiazol-5-ylboronic acid (275 mg, 1.1 mmol), tetrakis(triphenyl phosphene) palladium (0) (244 mg, 0.21 mmol), cesium carbonate (1.2 g, 3.69 mmol) in DMF: H2O (30 ml:10 mL) was stirred for 8 h at 80° C. Upon completion of the reaction, the solids were removed by filtration and the filtrate was diluted with water and extracted with ethyl acetate (2×100 mL). The combined organic layers were washed with water followed by brine, dried over Na₂SO₄ and concentrated under reduced pressure. The residue was purified by flash column chromatography (10% EtOAc/Hexane as eluent) to give ethyl-2-(3-benzo[c][1,2,5]thiadiazol-5-yl)-5-(4-(trifluoromethyl)benzyloxy)phenyl)-4-methyl pentanoate (160 mg) as an oil.

### Step 2

### 2-(3-benzo[c][1,2,5]oxazol-5-yl)-5-(4-(trifluoromethyl)benzyloxy)phenyl)-4-methyl pentanoic acid

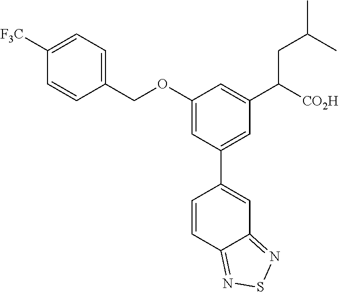

A solution of ethyl-2-(3-benzo[c][1,2,5]thiadiazol-5-yl)-5-(4-(trifluoromethyl)benzyloxy)phenyl)-4-methyl pentanoate (150 mg, 0.28 mmol), in MeOH/THF/H2O mixture (10 ml/10 ml/5 ml) and lithium hydroxide monohydrate (59.5 mg, 1.4 mmol) was stirred for 2 h at RT. Upon completion of the reaction, the volatiles were removed under reduced pressure and residue was diluted with water, acidified with 5% HCl solution and extracted with ethyl acetate (×2). The combined organic layers were washed with water, dried over Na2SO4, filtered and evaporated under reduced pressure. The residue was purified by flash column chromatography (using 1:1 EtOAc/Hexane as eluent) to give compound 2-(3-benzo[c][1,2,5]oxazol-5-yl)-5-(4-(trifluoromethyl)benzyloxy)phenyl)-4-methyl pentanoic acid (50 mg). 1HNMR (CDCl3, 400 MHz): 8.19 (s, 1H); 8.04 (d, 1H), 7.83 (d, 1H); 7.65 (d, 2H), 7.6 (d, 2H), 7.3 (s, 1H), 7.21 (s, 1H), 7.04 (s, 1H), 5.2 (s, 2H), 3.75 (t, 1H), 1.99 (m, 1H); 1.74 (m, 1H), 1.52 (m, 1H), 0.94 (d, 6H).

Measurement of Aβ in vitro

The Aβ peptide is proteolytically derived from a larger integral membrane amyloid precursor protein (APP). The production of Aβ is derived from proteolytic cleavages at its N- and C-termini within β-APP by the β and γ-secretase activities, respectively. Transfected cells overexpressing β-APP or its equivalent producing the Aβ peptide can be used to monitor the effects of synthetic compounds on the production of Aβ.

To analyze a compound's effects on the concentrations of the various products of the □-secretase cleavage activity, the A□ peptides, various methods known to a person skilled in the art are available. Examples of such methods, but not limited to, include mass-spectrometric identification as described by Wang et al, 1996, J. Biol. Chem. 271:31894-31902) or detection by specific antibodies using, for example, ELISA's.

Examples of such assays for measuring the production of A□total, A□40 and A□42 by ELISA include but are not limited to those described by Vassar et al., 1999, Science 286:735-741. Suitable kits containing the necessary antibodies and reagents for such an analysis are available, for example, but not limited to the Genetics Company, Wako, Covance, and Innogenetics. The kits are essentially used according to the manufacturers recommendations similar to the assay that is described by Citron et al., (1997) Nature Medicine 3:67-72 and the original assay described by Seubert et al., (1992) Nature 359:325-327.

Screening was carried out using the human embryonic kidney cell line HEK-293 overexpressing an amyloid precursor protein (APP) transgene grown in Pro-293a CDM media (BioWhittaker). Cells were grown to approximately 70-80% confluency subsequent to the addition of test compounds. The growth media was aspirated or removed, the cells washed, and replaced with 100 μl of compound, appropriately diluted in the serum free media from the dilution plate. The plates are then incubated for 16-18 hours at 37° C.

Conditioned Medium samples are removed for analysis/quantitation of the various A□ peptide levels by differential ELISA's as described in accompanying instructions to the kits. Those compounds examined which do not demonstrate any overt toxicity or non-specific inhibitory properties are investigated further for their A□ inhibitory effects and form the basis of medicinal chemistry efforts and to study the effect of the compounds in different experimental conditions and configurations.

A compound may have an IC50 for lowering A□42<10□M, in some cases compounds have an IC50 for lowering A□42<5□M, in further cases compounds may have an IC50 for lowering A□42<1□M and in still further cases compounds may may have an IC50 for lowering A□42<0.3□M

### Experimental Procedures for Rat Primary Cortical Culture-Based Abeta₁_{_{→}}_{42/1}_{_{→}}ₓ ELISAs

Rat primary neocortical cultures are established through the dissection of the neocortices from 10-12 E17 embryos harvested from time-pregnant CD (Sprague Dawley) rats (Charles River Laboratories). Following dissection, the combined neocortical tissue specimen volume is brought up to 5 mL with dissection medium (DM; 1×HBSS (Invitrogen Corp., cat#14185-052)/10 mM HEPES (Invitrogen Corp., cat# 15630-080)/1 mM Sodium Pyruvate (Invitrogen Corp., cat# 11360-070)) supplemented with 100 uL Trypsin (0.25%; Invitrogen Corp., cat# 15090-046) and 100 uL DNase I (0.1% stock solution in DM, Roche Diagnostics Corp., cat# 0104159), undergoing digestion via incubation at 37° C. for 10 minutes. Digested tissue is washed once in plating medium (PM; NeuroBasal (Invitrogen Corp., cat# 21103-049)/10% Horse Serum (Sigma-Aldrich Co., cat# H1138)/0.5 mM L-Glutamine (Invitrogen Corp., cat# 25030-081)), then resuspended in a fresh 10 mL PM volume for trituration. Trituration consists of 18 cycles with a 5 mL-serological pipet, followed by 18 cycles with a flame-polished glass Pasteur pipet. The volume is elevated to 50 mL with PM, the contents then passed over a 70 um cell-strainer (BD Biosciences, cat# 352350) and transferred directly to a wet-ice bath. The cell-density is quantified using a hemacytometer, and diluted to allow for the plating of 50000 cells/well/100 uL in pre-coated 96-well PDL-coated plates (Corning, Inc., cat# 3665). Cells are incubated for 4-5 hours at 37° C./5% CO₂, after which time the entire volume is exchanged to feeding medium (FM; NeuroBasal/2% B-27 Serum-free supplement (Invitrogen Corp., cat# 17504-044)/0.5 mM L-Glutamine/1% Penicillin-Streptomycin (Invitrogen Corp., cat# 15140-122)). The cultures undergo two 50% fresh FM exchanges, after 3 days in vitro (DIV3), and again at DIV7.

Human C-terminal recognition-site Abeta₁_{_{→}}₄₂ and Rat N-terminal recognition-site Abeta₁_{_{→}}ₓ capture-antibodies, diluted 1:300 in 0.05M Carbonate-Bicarbonate buffer (Sigma-Aldrich Co., C-3041), are plated at 100 uL/well on flat-bottomed F96 MicroWell™ (MaxiSorp™ surface) plates (Nalge Nunc International, cat# 439454), and incubated overnight at 4° C. Compounds to be screened are solubilized in dimethyl sulphoxide (DMSO, Sigma-Aldrich Co., cat# 15493-8), and further diluted in DMSO in an eight-point dose-response format. Into 96-well plates, dose-response compound dilutions (1000× the desired final concentration) are stamped out at 2 uL/well, in duplicate (up to 3 compounds/plate), as a daughter plate. In addition, DMSO and N—[N-(3,5-difluorophenacetyl)-L-alanyl]-S-phenylglycine t-butyl ester (DAPT), a gamma-secretase inhibitor (GSI), are incorporated as solvent and positive controls, respectively. With the assistance of liquid-handling automation, the compound daughter plate is diluted 1:500 with warmed FM, and two DIV8 culture plates are leveled to 60 uL/well, and immediately overlaid with 60 uL/well of the 2× diluted daughter plate. The plates are returned to the 37° C./5% CO₂-incubator for 24 hours.

Each capture-antibody ELISA plate undergoes 4× 250 uL/well Phosphate-buffered saline with 0.05% Tween®-20 SigmaUltra (PBS-T; Fluka, cat# 79383/Sigma-Aldrich Co., cat# P7949) washes. The ELISA plates are then overlaid with 120 uL/well PBS-T supplemented with 1% Bovine Serum Albumin Diluent/Blocking solution (BSA; Kirkegaard & Perry Laboratories (KPL), Inc., cat# 50-61-01) and incubate at room-temperature on an orbital shaker for a minimum of 2 hours.

Rat Abeta₁_{_{→}}₄₂ and rat Abeta₁_{_{→}}₄₀ peptide (American Peptide Co., cat# 62-0-84/62-0-86A) DMSO stock solutions are serially-diluted 1:2 in FM yielding a final concentration range of 0-500 pg/mL, to be plated on the respective ELISA plates for determination of the corresponding standard curve, from which concentrations of specific or total Abeta peptides in the presence of a particular drug concentration can be calculated. The conditioned medium from the duplicate culture plates are collected and combined into one round-bottom 96-well transfer plate which is incubated on wet-ice. The culture plates are rinsed once with 120 ul/well FM, and replenished immediately with 100 uL/well FM, being returned to the incubator for 10 minutes. Cell-viability is evaluated by adding 20 uL/well of warmed CellTiter 96® Aqᵤₑₒᵤₛ One Solution (MTS/PES; Promega Corp., cat# G3581), and returning the plates to the incubator for 30-90 minutes. Plate absorbance at 492 nm is read on a spectrophotometer, and from which, the ratio of absorbance of compound-treated cells to absorbance of solvent (DMSO)-treated control cells is calculated. The calculation of the corresponding EC₅₀ values is performed following non-linear curve-fitting using GraphPad Prism® software.

For each ELISA plate, a corresponding transfer-plate is created containing 120 uL/well of either the rat Abeta₁_{_{→}}₄₂ or rat Abeta₁_{_{→}}₄₀ peptide standard solutions, in duplicate, and 110-115 uL/well of the collected conditioned-medium plate, half designated for the Abeta₁_{_{→}}₄₂ ELISA, and the other half for the Abeta₁_{_{→}}ₓ ELISA. The ELISA plates undergo a second set of 4× 250 uL/well PBS-T washes, immediately followed by being overlaid with their designated transfer-plate. The ELISA plates incubate on an orbital-shaker for 16-18 hours at 4° C.

Detection antibody solution is prepared by diluting beta-Amyloid 17-24 (4G8) biotinylated monoclonal antibody (Covance, Inc., cat# SIG-39240-200) 1:1500 in PBS-T supplemented with 0.67% BSA. The ELISA plates undergo 4×250 uL/well PBS-T washes, and are overlaid with 100 uL/well of 4G8 diluted detection-antibody solution. The Abeta₁_{_{→}}₄₂ ELISA plates are incubated on an orbital-shaker at room-temperature for 90 minutes, the Abeta₁_{_{→}}ₓ ELISA plates for 60 minutes.

In order to conjugate the biotinylated monoclonal 4G8 antibody, following 4× 250 uL/well PBS-T washes, the ELISA plates undergo a one-hour incubation at 100 ul/well with a 1:15000 dilution of Streptavidin-HRP conjugate (Jackson ImmunoResearch Laboratories, Inc., cat# 016-030-0840) on an orbital-shaker at room temperature.

Following a final set of 4× 250 uL/well PBS-T washes, the ELISA plates are overlaid with 100 ul/well SureBlue 3,3′,5, 5′ —Tetramethylbenzidine (TMB) Microwell Peroxidase substrate solution (Kirkegaard & Perry Laboratories, Inc., cat# 52-00-02), protected from light, and incubate for 20-45 minutes at room temperature. At the point the desired level of development is attained, 100 ul/well of TMB Stop solution (Kirkegaard & Perry Laboratories, Inc., cat# 50-85-05) is added, and the plate thoroughly shaken in preparation for reading on a spectrophotometer. SureBlue TMB Microwell Substrate develops a deep blue color in the presence of a peroxidase-labeled conjugate, and turns yellow when stopped by acidification, allowing for plate absorbance at 450 nm to be read. From the calculation of the standard curve, the compound dose-response curves, normalized to DAPT performance, are plotted as % DMSO using GraphPad Prism® software, and the corresponding IC₅₀ values calculated.

Measurement of Aβ 42 in vivo

Compounds of the invention can be used to treat AD in mammal such as a human or alternatively in a validated animal model such as the mouse, rat, or guinea pig. The mammal may not be diagnosed with AD, or may not have a genetic predisposition for AD, but may be transgenic such that it overproduces and eventually deposits Aβ in a manner similar to that seen in the human. Additionally, non-transgenic animals may also be used to determine the biochemical efficacy of the compound, with an appropriate assay.

Compounds can be administered in any standard form using any standard method. For example, but not limited to, compounds can be in the form of liquid, tablets or capsules that are taken orally or by injection. Compounds can be administered at any dose that is sufficient to significantly reduce, for example, levels of Aβₜₒₜₐₗ or more specifically Aβ₄₂ in the blood plasma, cerebrospinal fluid (CSF), or brain.

To determine whether acute administration of the compound would reduce Aβ₄₂ levels in-vivo, two-three month old Tg2576 transgenic mice expressing APP₆₉₅ containing the “Swedish” variant could be used or any other appropriately validated transgenic model. This transgenic mouse displays spontaneous, progressive accumulation of β-amyloid (Aβ) in brain, eventually resulting in amyloid plaques within the subiculum, hippocampus and cortex. Animals of this age have high levels of Aβ in the brain but no detectable Aβ deposition. Mice treated with the compound would be examined and compared to those untreated or treated with vehicle and brain levels of soluble Aβ42 and total Aβ would be quantitated by standard techniques, for example, using ELISA. Treatments may be acute or sub-chronic and treatment periods may vary from hours to days or longer and can be adjusted based on the results of the biochemical endpoint once a time course of onset of effect can be established.

A typical protocol for measuring Aβ or Aβ₄₂ levels from in-vivo samples is shown but it is only one of many variations that could used to detect the levels of Aβ. For example, aliquots of compounds can be dissolved in DMSO (volume equal to 1/10th of the final formulation volume), vortexed and further diluted (1:10) with a 10% (w/v) hydroxypropyl β cyclodextrin (HBC, Aldrich, Ref N° 33, 260-7) solution in PBS, where after they are sonicated for 20 seconds.

Compounds may be administered as a single oral dose given three to four hours before sacrifice and subsequent analysis or alternatively could be given over a course of days and the animals sacrificed three to four hours after the administration of the final dose

Tg2576 mice can be anesthetized with a mixture of ketamine/xylazine (80/16 mg/kg intraperitoneally). When a deep level of anesthesia is reached, the mouse's head is secured in a stereotaxic frame. The skin on the back of the neck is retracted and the muscles on the back of the neck are removed to expose the cisterna magna. CSF is collected from the cisterna magna using a pulled 10 μl micropipette taking care not to contaminate the CSF with blood. The CSF is immediately diluted 1:10 in 1% 3-[3-cholamidopropyl)-dimethyl-ammonio]-1-propane sulfonate (CHAPS) [weight per volume in phosphate buffered saline (w/v in PBS)] containing protease inhibitors (PI's) (Complete, Mini protease inhibitor cocktail tablets-Roche), quick frozen in liquid nitrogen and stored at −80° C. until ready for biochemical analysis.

Blood is collected via cardiac puncture using a 25 gauge needle attached to a 1 ml syringe and was dispensed into a 0.6 ml microtainer tube containing ethylenediaminetetraacetic acid (EDTA). The blood was centrifuged immediately at 4° C. for 5 minutes at 1500×G. The resulting plasma was aliquoted into 0.5 ml microcentrifuge tubes, the aliquots are quick frozen in liquid nitrogen and are stored at −80° C.

The brain is removed after removing the skull and is rinsed with PBS. The cerebellum/brain-stem is removed, frozen, and retained for drug exposure analysis; the remaining brain section was quartered. The rear right quarter, which contained cortex and hippocampus, is weighed, frozen in liquid nitrogen and stored at −80° C. until ELISA analysis. The remaining brain tissue is frozen in liquid nitrogen and stored at −80° C.

For total Aβ or Aβ₄₀ analysis brain tissue is homogenized at a volume of 24 ml/g in cold 1% CHAPS containing protease inhibitors and the resulting homogenates are centrifuged for 1 hour at 100,000×g at 4° C. The supernatant is removed and transferred to a fresh tube and further diluted to 240 ml/g in CHAPS with protease inhibitors.

For Aβ₄₂ analysis brain tissue is homogenized at a volume of 50 ml/g in cold 1% CHAPS containing PI's. Homogenates were spun for 1 hour at 100,000×g at 4° C. The supernatant is removed and transferred to a fresh tube and further to diluted to a final volume 66.7 ml/g in 1% CHAPS with protease inhibitors.

To quantify the amount of human Aβ₄₂ in the soluble fraction of the brain homogenates, commercially available Enzyme-Linked-Immunosorbent-Assay (ELISA) kits can be used (h Amyloid β42 ELISA high sensitive, The Genetics Company, Zurich, Switzerland is just one of many examples). The ELISA is performed according to the manufacturer's protocol. Briefly, the standard (a dilution of synthetic Aβ1-42) and samples are prepared in a 96-well polypropylene plate without protein binding capacity (Greiner bio-one, Frickenhausen, Germany). The standard dilutions with final concentrations of 1000, 500, 250, 125, 62.5, 31.3 and 15.6 pg/ml and the samples are prepared in the sample diluent, furnished with the ELISA kit, to a final volume of 60 μl. Samples, standards and blancs (50 μl) are added to the anti-Aβ-coated polystyrol plate (capture antibody selectively recognizes the C-terminal end of the antigen) in addition with a selective anti-Aβ-antibody conjugate (biotinylated detection antibody) and incubated overnight at 4° C. in order to allow formation of the antibody-Amyloid-antibody-complex. The following day, a Streptavidine-Peroxidase-Conjugate is added, followed 30 minutes later by an addition of TMB/peroxide mixture, resulting in the conversion of the substrate into a colored product. This reaction is stopped by the addition of sulfuric acid (1M) and the color intensity is measured by means of photometry with an ELISA-reader with a 450 nm filter. Quantification of the A content of the samples is obtained by comparing absorbance to a standard curve made with synthetic Aβ1-42.

Similar analysis, with minor modification, can be carried out with CSF (Diluted 1:10 (for a final loading dilution of 1:100) in 1% CHAPS containing PI and plasma samples (Diluted 1:15 in 0.1% CHAPS [w/v in PBS]).

A compound may lower Aβ42 by >15%, in some cases compounds lower Aβ42 >25% and in further cases compounds may lower Aβ42 >40% relative to basal levels.

In Vivo Studies (rats)

Male Sprague Dawley rats from Harlan, 230-350 g, were used for studies. Fasted rats were dosed via oral gavage, with vehicle (15% Solutol HS 15, 10% EtOH, 75% Water) or compound, at a volume of 10 ml/kg. For PK studies, at fixed time points after dosing, the rats were euthanized with an excess of CO₂. Terminal blood was collected through cardiac puncture, mixed in EDTA tubes, immediately spun (3 min at 11,000 rpm at 4° C.), and snap frozen for plasma collection. A piece of frontal cortex was collected and snap frozen for compound level determination. For A-beta lowering studies, at a determined time point after dosing (Cmax if it is ≧3 hr), rats were euthanized as in the PK studies and plasma was collected as described above. Cerebellum was removed and saved for compound level determination, and the remaining brain was divided into 4 quadrants, snap frozen and saved to examine A-beta peptide levels. Solutol HS15 was purchased from Mutchler Inc.

Practitioners will also know that similar methods can also be applied to other species such as mice (including transgenic strains such as Tg2576), guinea pig, dog and monkey.

### Analysis of In Vivo Aβ Lowering Studies

Compounds of the invention can be used to treat AD in mammal such as a human or alternatively in a validated animal model such as the mouse, rat, or guinea pig. The mammal may not be diagnosed with AD, or may not have a genetic predisposition for AD, but may be transgenic such that it overproduces and eventually deposits Aβ in a manner similar to that seen in the human. Alternatively, non-transgenic animals may also be used to determine the biochemical efficacy of the compound, that is, the effect on the Aβ biomarker, with an appropriate assay.

Compounds can be administered in any standard form using any standard method. For example, but not limited to, compounds can be in the form of liquid, tablets or capsules that are taken orally or by injection. Compounds can be administered at any dose that is sufficient to significantly reduce, for example, levels of Aβₜₒₜₐₗ or more specifically Aβ₄₂ in the blood plasma, cerebrospinal fluid (CSF), or brain.

To determine whether acute administration of the compound would reduce Aβ₄₂ levels in-vivo, two-three month old non-transgenic Sprague-Dawley rats were used. Rats treated with the compound would be examined and compared to those untreated or treated with vehicle and brain levels of soluble Aβ₄₂ and Aβₜₒₜₐₗ would be quantitated by standard techniques, for example, using an immunoassay such as an ELISA. Treatments may be acute or sub-chronic and treatment periods may vary from hours to days or longer and can be adjusted based on the results of the biochemical endpoint once a time course of onset of effect can be established.

A typical protocol for measuring Aβ or Aβ₄₂ levels from in-vivo samples is shown but it is only one of many variations that could used to detect the levels of Aβ.

Compounds may be administered as a single oral dose given three to four hours before sacrifice and subsequent analysis or alternatively could be given over a course of days and the animals sacrificed three to four hours after the administration of the final dose

For total Aβ or Aβ₄₂ analysis brain tissue is homogenized in ten volumes of ice cold 0.4% DEA/50 mM NaCl containing protease inhibitors, e.g., for 0.1 g of brain 1 ml of homogenization buffer is added. Homogenization is achieved either by sonication for 30 seconds at 3-4 W of power or with a polytron homogenizer at three-quarters speed for 10-15 seconds. Homogenates (1.2 ml) are transferred to pre-chilled centrifuge tubes (Beckman 343778 polycarbonate tubes) are placed into a Beckman TLA120.2 rotor. Homogenates are centrifuged for 1 hour at 100,000 rpm (355,040×g) at 4° C. The resulting supernatants are transferred to fresh sample tubes and placed on ice (the pellets are discarded).

The samples are further concentrated and purified by passage over Waters 60 mg HLB Oasis columns according to the methods described (Lanz and Schachter (2006) J. Neurosci Methods. 157(1):71-81; Lanz and Schachter (2008). J. Neurosci Methods. 169(1):16-22). Briefly, using a vacuum manifold (Waters# WAT200607) the columns are attached and conditioned with 1 ml of methanol at a flow rate of 1 ml/minute. Columns are then equilibrated with 1 ml of water. Samples are loaded (800 μl) into individual columns (the Aβ will attach to the column resin). The columns are washed sequentially with 1 ml of 5% methanol followed by 1 ml of 30% methanol. After the final wash the eluates are collected in 13×100 mm tubes by passing 800 μl of solution of 90% methanol/2% ammonium hydroxide) over the columns at 1 ml/minute. The samples are transferred to 1.5 ml non-siliconized sample tubes are dried in a speed-vac concentrator at medium heat for at least 2 hours or until dry.

The dried samples are either stored at −80° C. or are used immediately by resuspending the pellets in 80 μl of Ultra-Culture serum-free media (Lonza) supplemented with protease inhibitors by vortexing for 10 seconds. Sixty microliters of each sample is transferred to a pre-coated immunoassay plate coated with an affinity purified rabbit polyclonal antibody specific to Aβ₄₂ (x-42). Sixty microliters of fresh supplemented ultraculture is added to the remaining sample and 60 microliters is transferred to a pre-coated and BSA blocked immunoassay plate coated with an affinity purified rabbit polyclonal antibody specific to total rodent Aβ (1-x). Additional standard samples of rodent Aβ/rodent Aβ₄₂ are also added to the plates with final concentrations of 1000, 500, 250, 125, 62.5, 31.3 and 15.6 pg/ml. The samples are incubated overnight at 4° C. in order to allow formation of the antibody-Amyloid-antibody-complex. The following day the plates are washed 3-4 times with 150 microliters of phosphate buffered saline containing 0.05% Tween 20. After removal of the final wash 100 μl of the monoclonal antibody 4G8 conjugated to biotin (Covance) diluted 1:1000 in PBS-T containing 0.67% BSA was added and the plates incubated at room temperature for 1-2 hours. The plates are again washed 3-4 times with PBS-T and 100 μl of a Streptavidin-Peroxidase-Conjugate diluted 1:10,000 from a 0.5 mg/ml stock in PBS-T contained 0.67% BSA is added and the plates incubated for at least 30 minutes. Following a final set of washes in PBS-T, a TMB/peroxide mixture is added, resulting in the conversion of the substrate into a colored product. This reaction is stopped by the addition of sulfuric acid (1M) and the color intensity is measured by means of photometry with an microplate reader with a 450 nm filter. Quantification of the Aβ content of the samples is obtained by comparing absorbance to a standard curve made with synthetic Aβ. This is one example of a number of possible measurable endpoints for the immunoassay which would give similar results.

### Pharmacokinetic Analysis

### Sample Preparation

Plasma samples and standards were prepared for analysis by treating with a 3× volume of acetonitrile containing 500 ng/mL of internal standard (a selected aryl propionic acid). Typically 150 μL of acetonitrile with internal standard was added to 50 μL of plasma. Acetonitrile was added first to each well of a 96-well Phenomenex Strata Impact protein precipitation filter plate followed by the addition of the plasma sample or standard. The filter plate was allowed to sit for at least 15 minutes at room temperature before a vacuum was applied to filter the samples into a clean 96-well plate.

If sample concentrations were observed or predicted to be greater than 1000 ng/mL, plasma samples were diluted with blank plasma 10-150 fold depending on the anticipated concentration and upper limit of quantitation of the analytical method.

Samples of frontal cortex or cerebellum were homogenized then treated in similar manner. To each brain sample, a 4× volume of PBS (pH 7.4) buffer was added along with a 15× volume of acetonitrile (containing internal standard) in a 2 mL screw-cap plastic tube. The tubes were then filled one third of the way with 1 mm zirconia/silica beads (Biospec) and placed in a Mini Bead Beater for 3 minutes. The samples were inspected and if any visible pieces of brain remained, they were returned to the Bead Beater for another 2-3 minutes of shaking. The resulting suspension was considered to be a 5-fold dilution treated with a 3× volume of acetonitrile (with internal standard). Calibration standards were prepared in 5-fold diluted blank brain homogenate and precipitated with a 3× volume of acetonitrile immediately after the addition of the appropriate spiking solution (see below). All brain standards and samples were allowed to sit for at least 15 minutes prior to filtering them through a Phenomenex Strata Impact protein precipitation filter plate into a clean 96-well plate.

Spiking solutions for plasma and brain calibration standards were prepared at concentrations of 0.02, 0.1, 0.2, 1, 2, 10, 20, 100 and 200 μg/mL in 50:50 acetonitrile/water. Calibration standards were prepared by taking 190 μL of blank matrix (plasma or brain homogenate) and adding 10 μL of spiking solution resulting in final concentrations of 1, 5, 10, 50, 100, 500, 1000, 5000 and 10,000 ng/mL.

### LC-MS/MS Analysis

Precipitated plasma and brain samples were analyzed by LC-MS/MS using a Shimadzu LC system consisting of two LC-10AD pumps and a SIL-HTc autosampler connected to an Applied Biosystems MDS/Sciex API 3200 QTRAP mass spectrometer.

For chromatographic separation, a Phenomenex Luna C-18 3 μM (2×20 mm) column was used with an acetonitrile-based gradient mobile phase. The two mobile phase components were:

Mobile phase A: water with 0.05% (v/v) formic acid and 0.05% (v/v) 5 N ammonium hydroxide.

Mobile phase B: 95:5 acetonitrile/water with 0.05% (v/v) formic acid and 0.05% (v/v) 5 N ammonium hydroxide.

The gradient for each analysis was optimized for the specific compound, but generally, the run started with between 0% and 40% of mobile phase B, ramped up to 100% of mobile phase B over 1-2 minutes, then held there for 2-3 minutes before returning to the initial conditions for 4 minutes to re-equilibrate.

The API 3200 QTRAP mass spectrometer was used in MRM mode with negative electrospray ionization. MRM transitions and mass spec settings were optimized for each compound.

Standard curves were created by quadratic or linear regression with 1/x*x weighting. Calibration standards were prepared 1-10,000 ng/mL, but the highest (and sometimes lowest) standards were often not acceptable for quantitation and only those standards with reasonable back-calculated accuracies were included in the calibration curve. Ideally, only standards with +/−15% of nominal concentration would be included in the fitted standard curve, but occasionally larger deviations were accepted after careful consideration.

Sample concentrations below the quantitation range were reported as “BQL”. Concentrations above the curve were usually re-run with larger sample dilutions.

1-93. (canceled)

94. A compound of formula (I), (II) and (III) 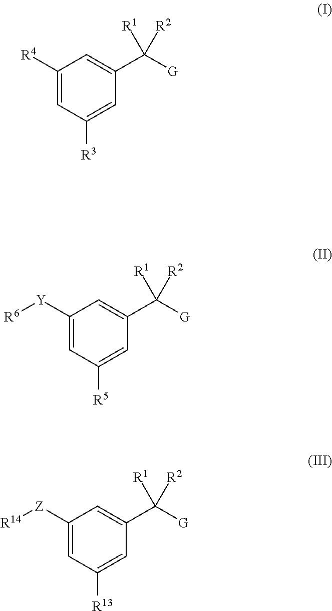
wherein G is a carboxylic acid or a tetrazole;
R¹ and R² are independently selected from H and R¹⁵
or
R¹ and R² are taken together to form a mono or bicyclic ring system having 4 to 11 ring atoms selected from C, N, O and S, provided that not more than 3 ring atoms in any single ring are other than C, wherein the mono or bicyclic ring system comprising of 4 to 11 ring atoms selected from C, N, O and S are optionally independently singly or multiply substituted with one or more substituents selected from, halogen, hydroxyl, amino, cyano or a C₁₋₄ alkyl substituent
Or
R₁ and R₂ are taken together to form a 3-7 membered cycloalkyl ring substituted with R₂₅ and R₂₆ where R₂₅ and R₂₆ are attached to the same carbon and taken together to form a second 3-7 membered cycloalkyl ring wherein each cycloalkyl is optionally multiply and independently substituted with halo, hydroxy, cyano, CF₃, C₁-C₄ alkyl;
R¹⁵ is selected from C₃-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl, any of which can be optionally substituted with one or more substituents independently selected from the group consisting of halo, N₃, CN, NO₂, oxo, OH, R⁹, OR⁹, SR⁹, S(O)R⁹, SO₂R⁹, CO₂R⁹, OC(O)R⁹, C(O)R⁹; C(O)N(R⁹R¹¹), SO₂N(R⁹R¹¹), S(O)N(R⁹R¹¹), N(R⁹)SO₂R¹¹, N(R⁹)SOR¹¹, N(R⁹)SO₂N(R¹⁰R¹¹), N(R⁹R¹¹), N(R⁹)C(O)R¹¹, N(R⁹)C(O)N(R¹¹R¹²), N(R⁹)CO₂R¹¹, and OC(O)N(R¹¹R¹²);
R³ is aryl and is optionally substituted with one or more substituents independently selected from halo, N₃, CN, NO₂, OH, R⁹, OR⁹, SR⁹, S(O)R⁹, SO₂R⁹, CO₂R⁹, OC(O)R⁹, C(O)R⁹; C(O)N(R⁹R¹¹), C(O)NH(R¹¹), C(O)NH(R⁹), SO₂N(R⁹R¹¹), SO₂NH(R⁹), SO₂NH(R¹¹), S(O)N(R⁹R¹¹), S(O)NH(R⁹), S(O)NH(R¹¹), NHSO₂R¹¹, N(R⁹)SO₂R¹¹, NHSOR11, N(R⁹)SOR¹¹, N(R⁹)SO₂N(R¹⁰R¹¹), NHSO₂N(R¹⁰R¹¹), N(R⁹)SO₂NH(R¹¹), N(R⁹)SO₂NH(R¹¹), N(R⁹R¹¹), NH(R⁹), NH(R¹¹), N(R⁹)C(O)R¹¹, NHC(O)R¹¹, N(R⁹)C(O)N(R¹¹R¹²), NHC(O)N(R¹¹R¹²), N(R⁹)C(O)NH(R¹¹), N(R⁹)C(O)NH(R¹²), N(R⁹)CO₂R¹¹, NHCO₂R¹¹, OC(O)N(R¹¹R¹²), OC(O)NH(R¹¹), OC(O)NH(R¹²);
R⁴ is selected from, C₁-C₆ alkyl, C₁-C₆ alkoxy, —O—(C₂-C₆ alkyl)-OH, —O—(C₂-C₆ alkyl)-O—(C₁-C₆ alkyl), heteroaryl, C₃-C₇ cycloalkyl, heterocycyl, C₁-C₆ alkynyl, —O—(C₁-C₄ alkyl)-Het² or R⁷—X— Where X is selected from —C₁-C₆ alkyl, —(C₀-C₆ alkyl)-O—(C₁-C₄ alkyl)-, —C(O)—, S(O)p-, —C(O)NR⁸—, N(R⁸)—C(O)—, —SO₂N(R⁸)—, —N(R⁸)—SO₂—, —O—C(O)NR⁸—, —N(R⁸)—C(O)—O—, —N(R⁸)—C(O)NR⁸—, —N(R⁸)—C(O)—N(R⁸)—, —C(O)—O—, —O—C(O)—, —O—C(O)—O—;
wherein the leftmost radical is attached to R⁷;
each alkyl group is optionally multiply substituted with groups independently selected from halo, —CF₃, —OCF₃, hydroxyl, amino, oxo or cyano;
p is an integer selected from 1 or 2;
R⁷ is selected from C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl;
R⁴ and R⁷ are independently and optionally multiply substituted with halo, N₃, CN, NO₂, OH, R⁹, OR⁹, SR⁹, S(O)R⁹, SO₂R⁹, CO₂R⁹, OC(O)R⁹, C(O)⁹, C(O)N(R⁹R¹¹), SO₂N(R⁹R¹¹), S(O)N(R⁹R¹¹), N(R⁹)SO₂R¹¹, N(R⁹)SOR¹¹, N(R⁹)SO₂N(R¹⁰R¹¹), N(R⁹R¹¹), N(R⁹)C(O)R¹¹, N(R⁹)C(O)N(R¹¹R¹²), N(R⁹)CO₂R¹¹, OC(O)N(R¹¹R¹²);
R⁸ is selected from H, C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl, wherein R⁸ is optionally multiply substituted with groups independently selected from halo, —CF₃, —OCF₃, hydroxyl, amino, oxo or cyano;
R⁹ is selected from C₁-C₇-alkyl, C₃-C₇ saturated cycloalkyl, (C₁-C₃)alkyl-(C₃-C₇)cycloalkyl, C₃-C₇ partially unsaturated cycloalkyl, saturated 4-8 membered heterocycle, partially unsaturated 4-8 membered heterocycle phenyl, heteroaryl, C₁-C₇-alkoxy and O—C₂-C₇—O—C₁-C₄ each of which is optionally with one or more substituents independently selected from the group F, CI, Br, I, CF₃, CN, OH, oxo, NH₂, NR¹¹R¹²;
R¹⁰, R¹¹, R¹² are independently selected from the group consisting of C₁-C₇ alkyl, C₁-C₇ alkoxy, O—C₂-C₇—O—C₁₋₄, 4-8 membered heterocycle; and C₃-C₇ cycloalkyl, phenyl or heteroaryl.
Each R¹⁰, R¹¹, R¹² group is optionally substituted with one or more substituents independently selected from the group consisting of F, CI, Br, I, CN, OH, oxo, amino and CF₃.
R⁵ is selected from heteroaryl, C₃-C₇ cycloalkyl, heterocycyl, wherein R⁵ is optionally substituted with one or more substituents independently selected from the group consisting of N₃, CN, NO₂, OH, R⁹, OR⁹, SR⁹, S(O)R⁹, SO₂R⁹, CO₂R⁹, OC(O)R⁹, C(O)R⁹, C(O)N(R⁹R¹¹), SO₂N(R⁹R¹¹), S(O)N(R⁹R¹¹), N(R⁹)SO₂R¹¹, N(R⁹)SOR¹¹, N(R⁹)SO₂N(R¹⁰R¹¹), N(R⁹R¹¹), N(R⁹)C(O)R¹¹, N(R⁹)C(O)N(R¹¹R¹²), N(R⁹)CO₂R¹¹, OC(O)N(R¹¹R¹²);
wherein Y is selected from a covalent bond, —O—, —C₁-C₆ alkyl, O—(C₁-C₆ alkyl)-, —(C₁-C₆ alkyl)-O—, —(C₁-C₆ alkyl)-O—(C₁-C₆ alkyl)-, —C(O)—, S(O)ₚ—, —O—C(R)(R)—, —C(O)NR⁸—, N(R⁸)—C(O)—, —SO₂N(R⁸)—, —N(R⁸)—SO₂—, —O—C(O)NR⁸—, —N(R)—C(O)—O—, —N(R⁸)—C(O)NR⁸—, —N(R⁸)—C(O)—N(R⁸)—, —C(O)—O—, —O—C(O)—, —O—C(O)—O— and wherein the leftmost radical is attached to R⁶;
p is 0, 1 or 2;
wherein each alkyl group is optionally multiply substituted with groups independently selected from halo, hydroxyl, amino, cyano oxo, and CF₃;
R⁶ is selected from C₁-C₆ alkyl, C₁-C₆ alkoxy, —O—(C₂-C₆ alkyl)-OH, —O—(C₂-C₆ alkyl)-O—(C₁-C₆ alkyl), aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl, wherein R⁶ is optionally substituted with one or more substituents independently selected from the group consisting of N₃, CN, NO₂, OH, R⁹, OR⁹, SR⁹, S(O)R⁹, SO₂R⁹, CO₂R⁹, OC(O)R⁹, C(O)R⁹, C(O)N(R⁹R¹¹), SO₂N(R⁹R¹¹), S(O)N(R⁹R¹¹), N(R⁹)SO₂R¹¹, N(R⁹)SOR¹¹, N(R⁹)SO₂N(R¹⁰R¹¹), N(R⁹R¹¹), N(R⁹)C(O)R¹¹, N(R⁹)C(O)N(R¹¹R¹²), N(R⁹)CO₂R¹¹, OC(O)N(R¹¹R¹²);
R¹³ is selected from halo, CN, CF₃, OCF₃, C₁-C₇ alkyl, C₁₋₇ alkoxy, —O—(C₂-C₇-alkyl)-O—C₁₋₄ alkyl), —O—(C₁-C₄ alkyl)-(C₃-C₇)cycloalkyl and —(C₁-C₄ alkyl)-(C₃-C₇)cycloalkyl each R¹³ is optionally multiply substituted with halo, cyano, CF₃ hydroxyl, oxo and amino;
R¹⁴ is selected from aryl, —(C₁-C₄ alkyl)-aryl, heteroaryl, —(C₁-C₄ alkyl)-heteroaryl, C₃-C₇ cycloalkyl, —(C₁-C₄ alkyl)-(C₃-C₇)cycloalkyl, heterocycyl, —(C₁-C₄ alkyl)-heterocycyl, wherein R¹⁴ is optionally substituted with one or more substituents independently selected from the group consisting of N₃, CN, NO₂, OH, R⁹, OR⁹, SR⁹, S(O)R⁹, SO₂R⁹, CO₂R⁹, OC(O)R⁹, C(O)R⁹, C(O)N(R⁹R¹¹), SO₂N(R⁹R¹¹), S(O)N(R⁹R¹¹), N(R⁹)SO₂R¹¹, N(R⁹)SOR¹¹, N(R⁹)SO₂N(R¹⁰R¹¹), N(R⁹R¹¹), N(R⁹)C(O)R¹¹, N(R⁹)C(O)N(R¹¹R¹²), N(R⁹)CO₂R¹¹, OC(O)N(R¹¹R¹²);
Z is selected from —O—, —C₁-C₆ alkyl, O—(C₁-C₆ alkyl)-, —(C₁-C₆ alkyl)-O—, —(C₁-C₆ alkyl)-O—(C₁-C₆ alkyl)-, —C(O)—, S(O)ₚ—, —C(O)NR⁸, N(R⁸)—C(O)—, —SO₂N(R⁸)—, —N(R⁸)—SO₂—, —O—C(O)NR⁸—, —N(R)—C(O)—O—, —N(R⁸)—C(O)NR⁸—, —N(R⁸)—C(O)—N(R⁸)—, —C(O)—, —O—C(O)—, —O—C(O)—O—, wherein the leftmost radical is attached to R¹⁴; and
p is 0, 1 or 2.

95. The compound of claim 94 wherein where R¹ and R² are taken together to form a mono or bicyclic ring system having 4 to 11 ring atoms selected from C, N, O and S, provided that not more than 3 ring atoms in any single ring are other than C. The R¹ and R² groups are optionally independently singly or multiply substituted with one or more substituents selected from, halogen, hydroxyl, amino, cyano or a C₁₋₄ alkyl substituent.

96. The compound of claim 94 wherein R³ is phenyl and is optionally substituted with one or more substituents independently selected from R⁹, OR⁹, SR⁹, S(O)R⁹, SO₂R⁹, CO₂R⁹, OC(O)R⁹, C(O)R⁹, N(R⁹)SO₂R¹¹ and SO₂N(R⁹R¹¹).

97. The compound of claim 94 wherein R⁴ is R⁷—X and X is selected from C(O)—, S(O)p-, —C(O)NR⁸—, N(R⁸)—C(O)—, —SO₂N(R⁸)—, —N(R⁸)—SO₂—, —O—C(O)NR⁸—, —N(R⁸)—C(O)—O—, —N(R⁸)—C(O)NR⁸—, —N(R⁸)—C(O)—N(R⁸)—, —C(O)—O— or —O—C(O)—.

98. A method for treating a neurodegenerative disorder comprising administering to a patient an effective amount of the compound of claim 94.

99. A method of treating a disease characterized by an elevated level of Aβ₄₂ comprising administering a therapeutically effective dose of the compound of claim 1.

100. A method of lowering Aβ₄₂ in a patient comprising administering a therapeutically effective dose of the compound of claim 1.

101. The method of claim 100 wherein the patient is suffering from Alzheimer's disease.
